(19) United States
(12) Patent Application Publication
(10) Pub. No.: US 2010/0316608 A1
(43) Pub. Date: Dec. 16, 2010

(54) Method of Determining A Response To Treatment With Immunomodulatory Composition

(76) Inventors: Vijayaprakash Suppiah, Marrickville (AU); David Booth, Chatswood (AU); Graeme Stewart, Palm Beach (AU); Jacob George, Sydney (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/714,336

(22) Filed: Feb. 26, 2010

(30) Foreign Application Priority Data
Jun. 15, 2009 (AU) AU2009902723

(51) Int. Cl. A61K 39/42; A61P 31/22; C12Q 1/68; G01N 33/566; C12Q 1/70

(52) U.S. Cl. 424/85.7; 435/6; 436/501; 435/5

(57) ABSTRACT

The present invention provides a method for accurately determining the likelihood that a subject will respond to treatment with an immunomodulatory composition comprising detecting one or more markers in a sample from the subject, wherein at least one markers is linked to a single nucleotide polymorphism (SNP) set forth in Table 1 or 3-5, and processes for selecting suitable subjects for therapy or for continued therapy, and for providing appropriate therapy to subjects, based on the assay results.

### FIELD OF THE INVENTION

The present invention is in the field of diagnostic and prognostic assays for medical conditions that are treated using an immunomodulatory composition, and improved therapeutic methods based on the diagnostic and prognostic assays of the invention.

### BACKGROUND TO THE INVENTION

Immunomodulatory compositions comprise drug compounds that act by modulating certain key aspects of the immune system in the treatment of viral diseases, neoplasias, Th1-mediated diseases, Th2-mediated diseases, or Th17-mediated diseases, substantially by modulating expression or secretion of one or more cytokines involved in autoimmunity and/or immune responses to infectious agents, or by modulating one of more components of a cytokine signalling pathway.

Cytokines may be interferons (IFNs, e.g., Type I IFNs such as IFN-α, IFN-β, or IFN-ω; or Type II IFNs such as IFN-γ; or Type III IFNs such as IFN-λ1, IFN-λ2, or IFN-λ3), interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, or IL-35), a tumor necrosis factor (e.g., TNF-α or TNF-β), or colony-stimulating factor (CSF). The IFNs generally assist immune responses by inhibiting viral replication within host cells, activating cytotoxic T cells and macrophages, increasing antigen presentation to lymphocytes, inducing resistance to viral and intracellular bacterial infections, and controlling tumors. Additionally, the Type III IFNs exert a regulatory effect on Th2 cells. Interleukins promote development and differentiation of T cells, B cells and hematopoietic cells. Tumor necrosis factors regulate cells of the immune systems to stimulate acute phase inflammatory responses, induce apoptotic cell death, inhibit tumorigenesis and inhibit viral replication. Although IFNs may be produced by a number of different cells, IFN-γ is produced predominantly by Th1 cells, and interleukins and TNF-α are produced by Th1 cells and/or Th2 cells.

Th1 cells and Th2 cells are effector T cells defined by their cytokine secretion profiles. Th1 cells mediate cellular immunity to protect against intracellular pathogens and immunogens via the actions of cytotoxic T lymphocytes and activated macrophages and complement-fixing and complement-opsonizing antibodies. Th1 cells produce IL-2, which stimulates growth and differentiation of T cell responses mediated by Th1 cells, as well as producing IFN-γ and TNF-β. On the other hand, Th2 cells mediate humoral immunity and allergic responses to protect against extracellular pathogens and antigens via the actions of B cells, mast cells and eosinophils. Th2 cells produce IL-3, IL-4, IL-5, and IL-10, which stimulate production of IgE antibodies, and also recruitment, proliferation, differentiation, maintenance and survival of eosinophils.

Certain Th1-mediated and Th2-mediated diseases are driven by disruption of the balance between Th1 cells and Th2 cells. The finely-tuned balance of Th1 and Th2 cells is regulated by cytokine secretion and, under normal circumstances, Th2 cells secrete IL-4 and IL-10 which down-regulate Th1 cells thereby regulating production of IFN-γ, TNF-β and IL-2. In particular, IL-10 is a potent inhibitor of Th1 cells. IFNs such as IFN-γ also drive Th1 cell production. Conversely, IL-4 drives Th2 cell production and IFN-γ inhibits Th2 cells. In Th1-mediated diseases e.g., multiple sclerosis (MS), rheumatoid arthritis (RA), Type I diabetes (IDDM) and scleroderma, delayed type hypersensitivity (DTH) occurs in those organ systems in which CD4⁺ Th1 cells are over-activated relative to Th2 cells. In MS, a Th1/Th2 imbalance in the central nervous system leads to proliferation of pro-inflammatory CD4⁺ Th1 cells, IFN-γ secretion, macrophage activation and consequential immune-mediated injury to myelin and oligodendrocytes, wherein the IFN-γ release in this case may also drive Th1 cell overproduction. In IDDM, a Th1/Th2 imbalance occurs in the thymus and periphery leading to progressive elimination of functional Th2 cells as autoreactive Th1 cells become activated and mediate pancreatic islet β-cell destruction. In localized scleroderma, the administration of IL-12 may restore Th1/Th2 immune balance. In contrast, Th2-mediated diseases e.g., Con A hepatitis, atopic dermatitis, asthma and allergy, are generally characterized by over-production of IgE antibodies and/or eosinophilia as a consequence of a Th1/Th2 imbalance. In Con A hepatitis, repeated injections of Con A shift an initial Th1 response to a Th2 and profibrogenic response, with over-production and secretion of IL-4, IL-10 and TGF-β in the liver activating natural killer T cells as part of an innate immune response thereby causing liver damage.

Th17 cells provide an effector arm distinct from Th1 and Th2 cells and, like Treg (iTreg), are regulated by TGF-β. Th17 cellular differentiation is important for host defense e.g., against bacteria and fungi, and poor regulation of Th17 cellular function is implicated in immune pathogenesis of autoimmune and inflammatory diseases.

Infections by a number of different viruses are treated using immunomodulatory compositions, including infections by human papillomaviruses such as HPV16, HPV6, HPV11; infections by herpesviruses such as HSV-1, HSV-2, VZV, HHV-6, HHV-7, HHV-8 (KSHV), HCMV and EBV; infections by picornaviruses such as the Coxsackie B viruses and encephalomyocarditis virus (EMCV); infections by flaviviruses such as the encephalitis viruses and hepatitis viruses e.g., hepatitis A virus, hepatitis B virus (HBV) and hepatitis C virus (HCV); arenaviruses such as those associated with a viral haemorrhagic fever; infections by togaviruses such as equine encephalitis viruses; infections by bunyaviruses such as Rift Valley fever virus, Crimean-Congo haemorrhagic fever virus, Hantaan hantavirus (HTNV) and Apeu virus (APEUV); infections by filoviruses such as Ebola virus and Marburg virus; infections by paramyxoviruses such as respiratory syncytial virus (RSV); infections by rhabdoviruses such as vesicular stomatitis virus (VSV); infections by orthomyxoviruses such as the influenza viruses e.g., influenza A virus (IAV); and infections by coronaviruses such as SARS-associated coronavirus. Neoplasias are also treated using immunomodulatory compositions e.g., HPV-associated cancer such as cervical intrapepithelial neoplasia, cervical carcinoma, vulvar intraepithelial neoplasia, penile intraepithelial neoplasia, perianal intraepithelial neoplasia; hepatocellular carcinoma; basal cell carcinoma, squamous cell carcinoma, actinic keratosis, and melanoma. Certain Th2-mediated diseases e.g., asthma, allergic rhinitis, atopic dermatitis, are also treated using immunomodulatory compositions.

Partly by virtue of the modulation of cytokines and cytokine signalling by immunomodulatory compositions, it is known to use cytokines per se as immunodulatory compositions.

For example, IFNs in general possess antiviral and anti-oncogenic properties, the ability to stimulate macrophage and natural killer cell activation, and the ability to enhance MHC class I and II molecules for presentation of foreign peptides to T cells. In many cases, the production of IFNs is induced in response to infectious agents, foreign antigens, mitogens and other cytokines e.g., IL-1, IL-2, IL-12, TNF and CSF. Thus, IFNs and IFN inducers have gained acceptance as therapeutic agents in the treatment of infections, neoplasias, Th1-mediated disease and Th2-mediated disease. IFNs are known to be used for treatment of infections by several positive-sense single-stranded RNA viruses i.e., (+) ssRNA viruses, including e.g., SARS-associated coronavirus, HBV, HCV, coxsackie B virus, EMCV, and for treatment of infections by several negative-sense single-stranded RNA viruses i.e., (−) ssRNA viruses, including e.g., Ebola virus, VSV, IAV, HTNV and APEUV (see e.g., De Clerq Nature Reviews 2, 704-720 (2004); Li et al, J. Leukocyte Biol., online publication DOI:10.1189/jlb.1208761 (Apr. 30, 2009). In such formulations, the IFN, especially IFN-α may be pegylated. Pegylated IFN-λ1 is currently in clinical trial for treatment of chronic HCV infection, and has been shown to be useful for protecting isolated cells against VSV, EMCV, HTNV, APEUV, IAV, HSV-1, HSV-2 and HBV, (see e.g., Li et al, J. Leukocyte Biol., online publication DOI:10.1189/jlb.1208761, Apr. 30, 2009). IFN-α is also known to be used in the treatment of certain lesions and neoplasias e.g., condylomata acuminata, hairy cell leukemia, Kaposi's sarcoma, melanoma, non-Hodgkin's lymphoma, however IFN-β has been shown to have potent anti-tumor activity against human astrocytoma/glioblastoma cells, whereas IFN-λ1 has been shown to have activity against glioblastoma cells, thymoma cells and fibrosarcoma cells, and IFN-λ2 has been shown to have activity against melanoma and fibrosarcoma cells (see e.g., Li et al, J. Leukocyte Biol., online publication DOI:10.1189/jlb.1208761, Apr. 30, 2009). It is also known to use IFN-β for treatment of relapsing forms of Th1-mediated diseases such as MS. IFN-λ2 has also been shown to protect against certain Th2-mediated diseases e.g., asthma and Con A-induced hepatitis (see e.g., Li et al, J. Leukocyte Biol., online publication DOI:10.1189/jlb.1208761, Apr. 30, 2009).

Since the expression of all IFN-λ proteins are induced by IFN-α, IFN-β and IFN-λ molecules e.g., Sirén et al., J. Immunol. 174, 1932-1937 (2005), Ank et al., J. Virol 80, 4501-4509 (2006) and Ank et al., J. Immunol. 180, 2474-2485 (2008), immunomodulatory compositions comprising IFN-α/β may act, at least in part, to induce IFN-λ proteins as effector molecules. The receptor complex for Type I IFNs consists of a heterodimeric IFNAR1/IFNAR2 complex, whereas Type III IFNs signal through a heterodimeric IL-28Rα/IL-10R2 receptor e.g., Li et al, J. Leukocyte Biol., online publication DOI:10.1189/jlb.1208761 (Apr. 30, 2009). IL-28Rα/IL-10R2 is expressed in far fewer contexts than IFNAR1/IFNAR2. This suggests that therapy using immunomodulatory compositions comprising IFN-α/β may be less specific than therapy using immunomodulatory compositions comprising IFN-λ. For example, administration of IFN-α/β may activate both receptor types i.e., directly via action of IFN-α/β on IFNAR1/IFNAR2 receptors and indirectly via induction of IFN-λ and subsequent action of IFN-λ on IL-28Rα/IL-10R2 receptors. Conversely, administration of IFN-λ is likely to activate selectively IL-28Rα/IL-10R2 receptors. Notwithstanding that this may be the case, all IFNs activate the Jak/STAT pathways and generally induce common interferon-stimulated genes (ISGs) that mediate the biological effects of IFNs e.g., Sirén et al., J. Immunol. 174, 1932-1937 (2005), Ank et al., J. Virol 80, 4501-4509 (2006), Ank et al., J. Immunol. 180, 2474-2485 (2008), and Li et al, J. Leukocyte Biol., online publication DOI:10.1189/jlb.1208761 (Apr. 30, 2009).

Various immunomodulatory compositions that induce IFN production e.g., poly(I)-poly(C), poly(I)-poly(C₁₂-U) or ampligen, and deazaneplanocin A, are also used in the treatment of infections by e.g., coxsackie B virus, Ebola virus and for certain flaviviruses and bunyaviruses that are amenable to treatment with IFNs (De Clerq Nature Reviews 2, 704-720 (2004). Immunomodulatory compounds may also exert their activity by activating Toll-like receptors (TLRs) to induce selected cytokine biosynthesis.

Immunomodulatory guanosine analogs, such as those having substituents at the 7-position and/or 8-position, e.g., Reitz et al., J. Med. Chem. 37, 3561-3578 (1994) Michael et al., J. Med. Chem. 36, 3431-3436 (1993) have been shown to stimulate the immune system, whilst 5′-O-proprionyl and 5′-O-butyryl esters of 2-amino-6-methoxy-9-(β-D-arabinofuranosyl)-9H-purine inhibit varicella zoster virus (VZV) e.g., U.S. Pat. No. 5,539,098 to Krenitsky. Other guanosine analogs, in particular 6-alkoxy derivatives of arabinofuranosyl purine, are useful for anti-tumor therapy e.g., U.S. Pat. No. 5,821,236 to Krenitsky. The 7-deazaguanosine analogs have been shown to exhibit antiviral activity in mice against a variety of RNA viruses, whereas 3-deazaguanine analogs have significant broad spectrum antiviral activity against certain DNA and RNA viruses e.g., Revankar et al., J. Med. Chem. 27, 1489-1496 (1984), and certain 7-deazaguanine and 9-deazaguanine analogs protect against a lethal challenge of Semliki Forest virus e.g., Girgis et al., J. Med. Chem. 33, 2750-2755 (1990). Selected 6-sulfenamide and 6-sulfinamide purine nucleosides are also disclosed in U.S. Pat. No. 4,328,336 to Robins as having demonstrated significant antitumor activity. Wang et al. (WO 98/16184) also disclose purine L-nucleoside compounds and analogs thereof were used to treat an infection, infestation, a neoplasm, an autoimmune disease, or to modulate aspects of the immune system. Guanosine analogs e.g., ribavirin and derivatives thereof e.g., acetate salts or ribavirin 5′-monophosphate or ribavirin 5′-diphosphate or ribavirin 5′-triphosphate or ribavirin 3′,5′-cyclic phosphate or the 3-carboxamidine derivative taribavirin (viramidine), 7-benzyl-8-bromoguanine, 9-benzyl-8-bromoguanine, and CpG-containing oligonucleotides, that shift the Th1/Th2 balance and are useful for the treatment of Th1-mediate or Th2-mediated disease depending upon their cytokine profiles. These compounds have been shown to elicit various effects on lymphokines IL-1, IL-6, IFN-α and TNF-α e.g., Goodman, Int. J. Immunopharmacol, 10, 579-588 (1988); U.S. Pat. No. 4,746,651; Smee et al., Antiviral Res. 15, 229 (1991); Smee et al., Antimicrobial Agents and Chemotherapy 33, 1487-1492 (1989). For example, 7-benzyl-8-bromoguanine and 9-benzyl-8-bromoguanine selectively inhibit Th1 cytokine production, specifically IL-2 and IFN-γ and therefore may be useful in the treatment of Th1-related autoimmune disease, which manifests activated T cells and overproduction of IFN-γ, and target leukemia and lymphoma cells, e.g., Poluektova et al., Int. J. Immunopharmacol. 21, 777-792 (1999). In contrast, ribavirin shifts an immune response from Th2 toward a Th1 cytokine profile, and is useful for treatment of Th2-mediated diseases. Ribavirin is useful in post-exposure prophylaxis of exposure to e.g., arenaviruses causing Lassa fever or Crimean-Congo hemorrhagic fever, HTNV, West Nile Virus, chronic HCV infection, AIV and RSV.

Various other immunomodulatory nucleotide analogs possess potent antiviral activity, and may restore p53 function in HPV-associated cancers e.g., cidofovir [(S)1-(3-hydroxy-2-phosphonylmethoxypropyl)cytosine, (HPMPC] e.g., Abdulkarin et al., Oncogene 21, 2334-2346, (2002). Cidofovir is used in the treatment of a number of viral conditions including HCMV-retinitis in AIDS patients and other HCMV infections and poxvirus infections.

Other classes of immunomodulatory compositions include small organic molecule imidazoquinoline amine derivatives e.g., U.S. Pat. Nos. 4,689,338 and 6,069,149; purine derivatives e.g., U.S. Pat. Nos. 6,028,076 and 6,376,501; imidazopyridine derivatives; e.g., U.S. Pat. No. 6,518,265; benzimidazole derivatives e.g., U.S. Pat. No. 6,387,938); adenine derivatives e.g., U.S. Pat. No. 6,376,501; and 3-β-D-ribofuranosylthiaz-olo[4,5-d]pyrimidine derivatives e.g., U.S. Pat. publication No. 200301994618. The immunosuppressive agent mycophenolate mofetil inhibits coxsackie B3 virus-induced myocarditis (see, e.g., Padalko et al., BMC Microbiol. 3, 25 et seq. (2003).

The list of immunomodulatory compositions provided herein is not exhaustive and a number of other compound classes are also known in the art e.g., in U.S. Pat. Nos. 5,446,153; 6,194,425; and 6,110,929.

The efficacy of immunomodulatory compositions for particular indications may be highly variable, and therapeutic outcome is likely to be influenced by host factors e.g., genotype including HLA haplotype effects, governing both innate and adaptive immune responses of subjects. Racial differences may also affect suitability of subjects for therapy with immunomodulators. The apparent failures of certain therapeutic agents as reported in the literature may be overstated in the absence of recognition of such genetic contributions. Clearly, any determination of therapeutic effect should optimally consider genotype effects.

Many immunomodulatory compositions also produce adverse side-effects, suggesting a benefit in limiting their application to contexts where therapeutic benefit outweighs detrimental effects. In addition to the favourable changes in the immune system that immunomodulatory compositions produce in therapy, imbalances occur. For example, the IFNs may cause, inter alia, psychiatric disorders, depression, anaphylxis, thrombocytopenia, seizure, cardiomyopathy, hepatotoxicity, flu-like symptoms, fever, fatigue, headache, muscle pain, convulsions, dizziness, erythema and immunosuppression through neutropenia, and interleukins e.g., IL-1, may cause dose-related fever and flu-like symptoms. In another example, guanosine analogs may be teratogenic with prolonged use. Accordingly, means for identifying and selecting those patients who are likely to respond to treatment with an immunomodulatory composition would provide a substantial therapeutic benefit to those patients that are either non-responders, low responders or relapsers, by avoiding inappropriate prescriptions to those patient classes and reducing the anxiety caused by subsequent treatment failure. More accurate prescription of drugs to responders also provides for reduced subsidies by health agencies. Moreover, for those conditions in which alternative therapies are available, such means may also provide for selection of the most appropriate therapy for a particular patient.

Notwithstanding the desirability of means for distinguishing patients according to their ability to respond to therapy with immunomodulatory compositions, the availability of reliable tests is limited. Many genetic tests have been proposed based on associations of single nuclear polymorphisms (SNPs) in small patient cohorts e.g., fewer than 100 subjects for which it is difficult to approach genome-wide significance. Well-characterized patient cohorts e.g., with respect to racial background, disease/infection parameters, therapeutic response, that are sufficiently large to permit associations approaching genome-wide significance to be determined are desirable for accurate prognosis. The use of multiple independent cohorts is also desirable for validation purposes. Depending upon the disease context, a suitable prognostic assay for treatment outcome to an immunomodulatory composition may require highly-significant associations, e.g., p value less than 1×10⁻³, to provide sufficient accuracy for clinical or commercial value. Similarly, correctly-matched comparison groups are required to derived meaningful associations. Functional significance, such as one or more effects of genotype on gene expression and/or therapeutic outcome, is also desirable for marker validation.

### SUMMARY OF THE INVENTION

### 1. Introduction

In work leading to the present invention, the inventors sought to ascertain to identify novel loci that might mediate viral clearance in individuals with chronic HCV infection who were administered immunomodulatory compositions comprising IFNs, specifically IFN-α. The inventors performed initial GWAS in a relatively large well-characterised Australian population of northern European ancestry and tested the most significantly associated SNPs in a much larger independent cohort of northern Europeans from the United Kingdom, Germany, Italy and Australia. The cohort size permitted the threshold for genome-wide significant association to be at p<1.6×10⁻⁷, such that SNPs having 1.6×10⁻⁷≦p≦1.0×10⁻⁴ could be considered to show a highly suggestive association with response to therapy, and SNPs having 1.0×10⁻⁴≦p<1.0×10⁻³ were considered to show a moderately suggestive association with response to therapy. Using these cut-off values, SNPs listed in the accompanying Tables were identified. The SNPs that the inventors have identified herein to have a high significance in their association with high response or low response to therapy are not believed to have been described previously for such an association.

Accordingly, in one example, the SNPs provided herein provide the means for accurately determining the likelihood that a subject will respond to therapy comprising an immunomodulatory composition.

As used herein, the terms “accurately determining” or “accurate prognosis” shall be taken to mean an association of a SNP, or a particular allele or genotype or haplotype with a high response (HR) or low response (LR) to therapy, or an association of a SNP, or a particular allele or genotype or haplotype with a non-response to therapy, or an association of a SNP, or a particular allele or genotype or haplotype with relapse, is significantly high (e.g., at p<10⁻³ or preferably at p<10⁻⁴ or more preferably p<10⁻⁵ or p<10⁻⁶ or p<10⁻⁷). For example, the significance of the association means that there is a probability of a correct prognosis in at least 90% or at least 95% or at least 96% or at least 97% or at least 98% or at least 99% or more than 99% of cases in a population. In this context, the term “population” means a test population of greater than 100 matched individuals or greater than 200 matched individuals or greater than 300 matched individuals or greater than 400 matched individuals or greater than 500 matched individuals. By “matched” is meant that the individuals of the test population have similar or near-identical age, BMI, viral titer, and treatment regime. For practical purposes, the present invention also provides for accurate prognosis in a “real world” population of individuals suffering from the same medical condition e.g., individuals suffering from the same condition that are at least matched with respect to ethnicity. By way of explanation and without limitation, one example of the invention provides for accurate prognosis of treatment for primary or chronic HCV infection in a population of Caucasion patients.

As used herein, the term “immunomodulatory composition” shall be taken in its broadest context to mean a composition comprising one or more compounds capable of modulating expression or secretion of one or more cytokines involved in autoimmunity and/or immune responses to infectious agents, or by modulating one or more components of a cytokine signalling pathway. The term “compound” in this context includes a protein, small molecule, antibody molecule, or nucleic acid e.g., RNAi, antisense RNA, ribozyme or siRNA.

The present invention has clear application for the accurate prognosis of a response to any therapy comprising administration of an “immunomodulatory composition” that is known to be used and/or known to be useful in the treatment of a viral infection and/or neoplasia and/or Th1-mediated disease and/or Th2-mediated disease.

For example, the invention is suitable for accurate prognosis of a response to therapy comprising administration of an “immunomodulatory composition” for treatment of Th1-mediated disease and/or Th2-mediated disease e.g., one or more conditions selected individually or collectively from the group consisting of multiple sclerosis (MS), rheumatoid arthritis (RA), Type I diabetes (IDDM), scleroderma, Con A hepatitis, atopic dermatitis, asthma, allergic rhinitis and allergy. Alternatively, or in addition, the invention is suitable for accurate prognosis of a response to therapy comprising administration of an “immunomodulatory composition” for treatment of one or more infections by viruses selected individually or collectively from the group consisting of human papillomaviruses (e.g., papillomavirus(es) selected from HPV16, HPV6 and HPV11), herpes viruses (e.g., herpes virus(es) selected from HSV-1, HSV-2, VZV, HHV-6, HHV-7, HHV-8 (KSHV), HCMV and EBV), picornaviruses (e.g., picornavirus(es) selected from Coxsackie B virus(es) and EMCV), flaviviruses (e.g., flavivirus(es) selected from encephalitis virus(es) and hepatitis virus(es) such as HAV and/or HBV and/or HCV), arenaviruses (arenavirus(es) associated with a viral haemorrhagic fever); togaviruses (togavirus(es) selected from equine encephalitis viruses), bunyaviruses (e.g., bunyavirus(es) selected from Rift Valley fever virus, Crimean-Congo haemorrhagic fever virus, HTNV and APEUV), filoviruses (e.g., filovirus(es) selected from Ebola virus and Marburg virus), paramyxoviruses (e.g., RSV), rhabdoviruses (e.g., VSV), orthomyxoviruses (e.g., influenza viruses such as IAV), and coronaviruses (e.g., SARS-associated coronavirus, “SARS-CoV”). For example, the invention provides means for prognosis of a response to therapy comprising administration of an “immunomodulatory composition” for treatment of one or more infections by hepatitis virus(es), such as HAV and/or HBV and/or HCV, and especially HCV. Alternatively, or in addition, the invention is suitable for accurate prognosis of a response to therapy comprising administration of an “immunomodulatory composition” for treatment of one or more neoplasias or pre-cancerous conditions, such as neoplasia(s) and pre-cancerous condition(s) selected individually or collectively from the group consisting of HPV-associated cancer (e.g., cervical intrapepithelial neoplasia and/or cervical carcinoma and/or vulvar intraepithelial neoplasia and/or penile intraepithelial neoplasia and/or perianal intraepithelial neoplasia), hepatocellular carcinoma, basal cell carcinoma, squamous cell carcinoma, actinic keratosis, melanoma, hairy cell leukemia, Kaposi's sarcoma, non-Hodgkin's lymphoma, astrocytoma, glioblastoma, thymoma, fibrosarcoma.

In another example, the SNPs provided herein provide the means for accurately determining the likelihood that a subject will respond to therapy comprising of an immunomodulatory composition comprising IFN.

Unless the context requires otherwise, the term “IFN” as used herein shall be taken to include any known interferon molecule e.g., IFN-α, IFN-β, IFN-ω, IFN-γ, IFN-λ1, IFN-λ2, or IFN-λ3, a composition comprising a plurality of any interferon molecules e.g., two or more molecules selected from IFN-α, IFN-β, IFN-ω, IFN-γ, IFN-λ1, IFN-λ2 and IFN-λ3, a composition comprising one or more derivatives of an interferon molecule e.g., a pegylated interferon, and mixtures of said one or more derivatives with one or more non-derivative interferon molecules.

For example, the present invention has clear application for the prognosis of a response to any therapy comprising administration of “IFN” that is known to be used and/or known to be useful in the treatment of a viral infection and/or neoplasia and/or Th1-mediated disease and/or Th2-mediated disease. For example, the invention is useful for prognosis of a response to an infection treatable by “IFN”, wherein the infection is by one or more ssRNA viruses, i.e., an infection by one or more (+) ssRNA viruses and/or an infection by one or more (−) ssRNA viruses, such as SARS-associated coronavirus (SARS-CoV), HBV, HCV, coxsackie B virus, EMCV, Ebola virus, VSV, IAV, HTNV, or APEUV, and/or one or more double-stranded DNA viruses such as HSV-1 or HSV-2. Alternatively, or in addition, the invention is useful for prognosis of a pre-cancerous lesion or neoplasia treatable by “IFN” e.g., a pre-cancerous lesion or neoplasia selected from the group consisting of condylomata acuminata, hairy cell leukemia, Kaposi's sarcoma, melanoma, non-Hodgkin's lymphoma, astrocytoma, glioblastoma, thymoma and fibrosarcoma. Alternatively, or in addition, the invention is useful for prognosis of a Th1-mediated disease or Th2-mediated disease treatable by “IFN” e.g., a disease selected from the group consisting of MS, asthma and Con A-induced hepatitis.

In another example, the SNPs provided herein provide the means for accurately determining the likelihood that a subject will respond to therapy comprising an immunomodulatory composition comprising guanosine analog(s).

Unless the context requires otherwise, the term “guanosine analog” as used herein shall be taken to include any known guanosine analog, a composition comprising a plurality of guanosine analogs, a composition comprising one or more derivatives of one or more guanosine analogs and mixtures of said one or more derivatives with one or more non-derivative guanosine analogs. Preferred guanosine analogs in this context are those compounds that are capable of modulating levels of Th1 and/or Th2 cells, or that have antiviral and/or anti-cancer activity. Exemplary guanosine analogs are selected from ribavirin, viramidine, 7-benzyl-8-bromoguanine, 9-benzyl-8-bromoguanine, and CpG-containing oligonucleotide(s), and derivative(s), salt(s), solvate(s) and hydrate(s) thereof e.g., ribavirin 5′-monophosphate, ribavirin 5′-diphosphate, ribavirin 5′-triphosphate, and ribavirin 3′,5′-cyclic phosphate.

In another example, the SNPs provided herein provide the means for accurately determining the likelihood that a subject will respond to therapy comprising an immunomodulatory composition comprising IFN and guanosine analog(s).

As will be known to the skilled artisan, the SNPs identified in Table 1 hereof comprise allelic variants that are associated with a high response (HR) to therapy or a low response (LR) to therapy. Accordingly, the present invention clearly encompasses the use of any HR allele and/or their LR allele set forth in Table 1, and any combination thereof e.g., a specific haplotype, for determining the likelihood that a subject will respond to therapy comprising an immunomodulatory composition as described herein. The HR and LR alleles of untagged SNPs in Table 1 can be readily determined following the exemplified methods and disclosure elsewhere in this specification. Accordingly, the present invention also encompasses the use of any other HR allele and/or their LR allele of a polymorphic locus set forth in Table 1, and any combination thereof e.g., a specific haplotype, for determining the likelihood that a subject will respond to therapy comprising an immunomodulatory composition as described herein.

The present invention also provides the first associations of particular regions of the human genome with treatment outcome. By virtue of the rigor applied by the inventors to selecting the SNPs of the invention that provide accurate prognosis, the value of these regional chromosomal associations is high. The present invention also encompasses the use of any chromosomal region linked to a polymorphic locus set forth in Table 1, and the use of any chromosomal region linked to a HR allele and/or LR allele of a polymorphic locus set forth in Table 1, and any combination thereof e.g., a specific haplotype, for determining the likelihood that a subject will respond to therapy comprising an immunomodulatory composition as described herein. For example, the chromosomal region(s) may be employed for accurate prognosis.

In one example, such chromosomal regions are selected individually or collectively from the group consisting of: a region at 1p35; a region between about 3p21.2 and about 3p21.31; a region between about 3p24.3 and about 3p25.1; a region at about 4q32; a region at about 4p13; a region at about 4p16.1; a region between about 6p12.2 and about 6p12.3; a region between about 6p21.33 and about 6p22; a region between about 6p22.1 and about 6p22.2; a region at about 6q13; a region at about 6q22.31; a region between about 8q12.2 and about 8q13.1; a region between about 9q22.1 and about 9q22.2; a region between about 10q26.2 and about 10q26.3; a region at about 11q21; a region at about 11q22.3; a region between about 14q22.1 and 14q22.2; a region between about 16q23.1 and about 16q23.2; a region between about 16p11.2 and about 16p12.1; a region at about 19q13.13; and a region between about 20q13.12 and about 20q13.13.

In another example, such chromosomal regions are linked to genes not previously known to have an association with therapeutic outcome in treatment of a condition with an immunomodulatory agent as described herein e.g., chromosomal regions selected individually or collectively from the group consisting of: a region at about 1p35; a region between about 3p21.2 and about 3p21.31; a region between about 3p24.3 and about 3p25.1; a region at about 4q32; a region at about 4p13; a region at about 4p16.1; a region between about 6p12.2 and about 6p12.3; a region between about 6p21.33 and about 6p22; a region between about 6p22.1 and about 6p22.2; a region at about 6q13; a region at about 6q22.31; a region between about 8q12.2 and about 8q13.1; a region between about 9q22.1 and about 9q22.2; a region between about 10q26.2 and about 10q26.3; a region at about 11q21; a region between about 14q22.1 and 14q22.2; a region between about 16q23.1 and about 16q23.2; a region between about 16p11.2 and about 16p12.1; a region at about 19q13.13; and a region between about 20q13.12 and about 20q13.13.

In another example, a chromosomal region disclosed herein is suitable for determining the likelihood that a subject will respond to therapy comprising an immunomodulatory composition comprising IFN and/or guanosine analog(s) as described according to any example hereof.

The data provided herein also demonstrate that certain SNPs identified by the inventors are positioned within or near to structural genes. For example, Table 1 hereof indicates significant associations between several SNPs that are linked to genes and treatment outcome.

By “linked to a gene” or “linked to genes” is meant that the SNPs are positioned within the structural gene i.e., intron or exon regions, or within a 5′-upstream or 3′-downstream region of the structural gene and in sufficient proximity to the structural gene so as to be in linkage disequilibrium with it and/or so as to have an association with expression of the structural gene. A SNP will also be considered to be linked to a gene if a physical or genetic marker e.g., another SNP, that is positioned more distally from a 5′-terminus or 3′-terminus of the corresponding structural gene portion than said SNP is in linkage disequilibrium with the structural gene and/or associated with expression of the structural gene. For example, a haplotype block comprising markers in linkage disequilibrium will be linked to a gene when one or more alleles of the haplotype block are linked to the gene. SNPs are generally, but not necessarily, linked to a gene if they are positioned within 5 kb of the 5′-end or 3′ end of the gene.

By following such criteria, the haplotype block identified and characterized by the inventors for the IFN-λ3 gene (Table 6), and expression data (FIG. 1) demonstrating that expression of IFN-λ2 and IFN-λ3 is reduced in carriers of the LR allele i.e., the G allele, of rs8099917 relative to carriers of the corresponding HR allele i.e., the T allele, indicate that all of the chromosome 19 SNPs presented in Table 1 are definitely linked to the IFN-λ3 gene, with the possible exception of rs4803224, rs12980602 and rs10853728. The excluded SNPs under these criteria are more distal than rs8099917 from the structural gene region i.e., encoding IFN-λ3. Thus, the present invention also provides SNPs linked to IFN-λ3 that are associated with treatment outcome e.g., in the 5′-upstream region or an intron or an exon or the 3′-downstream region. Similarly, the present invention provides SNPs linked to SULF-2 and/or WWOX-1 and/or RTFN-1 and/or CACNA2D3 and/or CASP-1 and/or RIMS-1 and/or PKHD-1 and/or IL21R and/or NPS that are associated with treatment outcome e.g., in one or more introns of any one or more of those genes. By virtue of the rigor applied by the inventors to selecting the SNPs of the invention that provide accurate prognosis, the value of these intragenic associations is high.

Accordingly, the present invention also encompasses the use of a gene or fragment thereof linked to a polymorphic locus set forth in Table 1, and the use of any gene linked to a HR allele and/or LR allele of a polymorphic locus set forth in Table 1, and any combination thereof e.g., a specific haplotype, for determining the likelihood that a subject will respond to therapy comprising an immunomodulatory composition as described herein. For example, the gene or fragment may be employed for accurate prognosis. By “fragment” in this context, is meant a portion of a gene of sufficient length to be useful for detection of gene expression associated with the polymorphism and/or of sufficient length to directly identify the polymorphism e.g., in a platform suitable for identifying SNPs as described herein.

In one example, the present invention encompasses the use of a gene selected individually or collectively from the group consisting of IFN-λ3, SULF-2, WWOX-1, RTFN-1, CACNA2D3, CASP-1, RIMS-1, PKHD-1, IL21R and NPS for determining the likelihood that a subject will respond to therapy comprising an immunomodulatory composition as described herein. In another example, such genes are not previously known to have an association with therapeutic outcome in treatment of a condition with an immunomodulatory agent as described herein e.g., IFN-λ3, SULF-2, WWOX-1, RTFN-1, CACNA2D3, RIMS-1, PKHD-1, IL21R and NPS. In another example, a gene disclosed herein is suitable for determining the likelihood that a subject will respond to therapy comprising an immunomodulatory composition comprising IFN and/or guanosine analog(s) as described according to any example hereof. Clearly, these examples extend to the use of gene fragments of one or more of the stated genes.

The data support the inventors' conclusion that variations in 19q13.13 between position 44,420,000 and position 44,440,000 and more specifically between about position 44,423,000 and about position 44,436,000, such as those linked to the IFN-λ3 (IL28B) gene, contribute to the variation in response to therapy with an immunomodulatory composition as described according to any example hereof. The instant association between variations in the IL28B gene is sufficiently-strong to indicate that genotypes in 19q13.13 between position 44,425,000 and position 44,436,000, especially IFN-λ3 (IL-28B) genotypes (Tables 4 and 5), can be used to predict drug responses. The haplotype effect of the LR allele for rs8099917 and linkage disequilibrium across SNPs linked to the IFN-λ3 (IL-28B) gene (Table 6) support this conclusion. Finally, the correlation between the LR allele at rs8099917 in this haplotype block and low expression of the IFN-λ2 and IFN-λ3 genes also demonstrates functional significance of the associations described herein, and especially with respect to IFN therapy.

Accordingly, in yet another example, the IFNλ3 gene or a fragment thereof is particularly suitable for determining the likelihood that a subject will respond to therapy comprising an immunomodulatory composition e.g., IFN and/or guanosine analog(s) as described according to any example hereof. Because the SNPs described herein are within the 5′-upstream region, introns, exons, or the 3′-downstream region, any gene fragments encompassing any one or more of these regions are also useful for prognosis of treatment outcome, subject to such fragments being of sufficient length to be useful for detection of gene expression associated with a polymorphism and/or of sufficient length to directly identify a polymorphism. Fragments within the 5′-upstream region and/or within an intron and/or within an exon and/or within the 3′-downstream region of the IFNλ3 gene are also useful. The present invention also encompasses the use of any polymorphic locus of an IFNλ3 gene e.g., as set forth in Table 1, and the use of any HR allele and/or LR allele of said polymorphic locus set forth in Table 1, and any combination thereof e.g., a specific haplotype such as a haplotype comprising alleles of rs12980275, rs8105790, rs8103142, rs10853727, rs8109886 and rs8099917, for determining the likelihood that a subject will respond to therapy comprising an immunomodulatory composition as described herein.

The known disease associations of the genes identified herein to have linked SNPs associated herein with treatment outcome to immunomodulatory composition(s) indicates further application of one or more IFNs in the treatment of diseases not known to be treatable with immunomodulatory composition(s) e.g., carcinoma and infection by gram-negative bacteria. For example, SULF-2 is associated with asthma, liver cancer and breast cancer; WWOX-1 and CACNA2D3 are tumor-suppressor genes that are associated with various cancers, including breast cancer, lung cancer, adenocarcinoma, squamous cell carcinoma, ovarian cancer; CASP-1 is associated with infection by gram-negative bacteria e.g., Escherichia coli and Salmonella typhimurium; and PKHD-1 is associated with polycystic kidney disease, post-transplant diabetes in subjects having polycystic kidney disease and poor clearance of HCV in post-transplant patients.

Accordingly, in yet another example, IFN is used in the preparation of a medicament for the treatment of a carcinoma e.g., a carcinoma of breast, a carcinoma of liver, a carcinoma of the lung, a carcinoma of the ovary, adenocarcinoma, or squamous cell carcinoma.

In yet another example, IFN is used in the preparation of a medicament for the treatment of infection by a gram-negative bacterium e.g., Escherichia coli or Salmonella typhimurium.

In yet another example, IFN is used in the preparation of a medicament for the treatment of polycystic kidney disease or complication arising therefrom e.g., post-transplant diabetes.

The strong associations between response to IFN-α in the treatment of HCV infection and polymorphisms in the IFN-λ3 gene also suggest that IFN-λ3 and/or the structurally similar IFN-λ2 have general utility in the treatment of medical conditions known to be treated using IFN-α/β, especially HCV infection.

Accordingly, in yet another example, IFN-λ2 and/or IFN-λ3 is used in the preparation of a medicament for the treatment of a medical condition known to be treated using IFN-α/β e.g., a viral infection and/or neoplasia and/or Th1-mediated disease and/or Th2-mediated disease such as an infection by one or more (+) ssRNA viruses and/or an infection by one or more (−) ssRNA viruses, such as SARS-associated coronavirus (SARS-CoV), HBV, HCV, coxsackie B virus, EMCV, Ebola virus, VSV, IAV, HTNV, or APEUV, and/or an infection by one or more double-stranded DNA viruses such as HSV-1 or HSV-2, and/or a pre-cancerous lesion or neoplasia such as a sarcoma or lymphoma or leukemia (e.g., condylomata acuminata, hairy cell leukemia, Kaposi's sarcoma, melanoma, non-Hodgkin's lymphoma, astrocytoma, glioblastoma, thymoma or fibrosarcoma) and/or a disease selected from the group consisting of MS, asthma and Con A-induced hepatitis.

In a preferred example, IFN-λ2 is used in the preparation of a medicament for the treatment of infection by HCV, e.g., a primary infection or chronic infection.

In a particularly preferred example, IFN-λ3 is used in the preparation of a medicament for the treatment of infection by HCV, e.g., a primary infection or chronic infection.

The known disease associations of the genes identified herein to have linked SNPs associated herein with treatment outcome to immunomodulatory composition(s) indicates further application of the invention to the prediction of treatment outcome for diseases that are not necessarily known to respond to immunomodulatory composition(s).

Accordingly, in yet another example, a tumor-suppressor gene e.g., WWOX-1 and/or CACNA2D3, or a fragment of a tumor suppressor gene is suitable for determining the likelihood that a subject will respond to an immunomodulatory composition e.g., IFN and/or guanosine analog(s) as described according to any example hereof, in the treatment of cancer or a pre-cancerous condition e.g., breast cancer, lung cancer, adenocarcinoma, squamous cell carcinoma, or ovarian cancer.

In yet another example, the SULF-2 gene or a fragment thereof is suitable for determining the likelihood that a subject will respond to an immunomodulatory composition e.g., IFN and/or guanosine analog(s) as described according to any example hereof, in the treatment of asthma, cancer or a pre-cancerous condition, e.g., liver cancer or breast cancer.

In yet another example, a PKHD-1 gene or a fragment thereof is suitable for determining the likelihood that a subject will respond to an immunomodulatory composition e.g., IFN and/or guanosine analog(s) as described according to any example hereof, in the treatment of polycystic kidney disease or complication arising therefrom e.g., post-transplant diabetes.

In yet another example, the CASP-1 gene or a fragment thereof is suitable for determining the likelihood that a subject will respond to an immunomodulatory composition e.g., IFN and/or guanosine analog(s) as described according to any example hereof, in the treatment of an infection with a gram negative bacterium such as Escherichia coli or Salmonella typhimurium.

### 2. Specific Embodiments

The scope of the invention will be apparent from the claims as filed with the application that follow the examples. The claims as filed with the application are hereby incorporated into the description. The scope of the invention will also be apparent from the following description of specific embodiments.

In one example, the present invention provides a method for accurately determining the likelihood that a subject will respond to treatment with an immunomodulatory composition, said method comprising detecting one or more markers in a sample from the subject, wherein at least one marker is linked to a single nuclear polymorphism (SNP) set forth in Table 1 or comprises a SNP set forth in Table 1 or is encoded by nucleic acid comprising a SNP set forth in Table 1 or linked to a SNP set forth in Table 1, and wherein detection of said one or more markers is indicative of the likely response of the subject to treatment with said composition.

For example, at least one marker is linked to a SNP set forth in Table 3 or comprises a SNP set forth in Table 3 or is encoded by nucleic acid comprising a SNP set forth in Table 3 or linked to a SNP set forth in Table 3, or at least one marker is linked to a SNP set forth in Table 4 or 5 or comprises a SNP set forth in Table 4 or 5 or is encoded by nucleic acid comprising a SNP set forth in Table 4 or 5 or linked to a SNP set forth in Table 4 or 5.

Alternatively, or in addition, at least one marker is contained within a chromosomal region are selected from the group consisting of: a region at 1p35; a region between about 3p21.2 and about 3p21.31; a region between about 3p24.3 and about 3p25.1; a region at about 4q32; a region at about 4p13; a region at about 4p16.1; a region between about 6p12.2 and about 6p12.3; a region between about 6p21.33 and about 6p22; a region between about 6p22.1 and about 6p22.2; a region at about 6q13; a region at about 6q22.31; a region between about 8q12.2 and about 8q13.1; a region between about 9q22.1 and about 9q22.2; a region between about 10q26.2 and about 10q26.3; a region at about 11q21; a region at about 11q22.3; a region between about 14q22.1 and 14q22.2; a region between about 16q23.1 and about 16q23.2; a region between about 16p11.2 and about 16p12.1; a region at about 19q13.13; and a region between about 20q13.12 and about 20q13.13.

Alternatively, or in addition, at least one marker is linked to a gene selected from the group consisting of IFN-λ3, SULF-2, WWOX-1, RTFN-1, CACNA2D3, CASP-1, RIMS-1 and PKHD-1 or is contained within a gene selected from the group consisting of IFN-λ3, SULF-2, WWOX-1, RTFN-1, CACNA2D3, CASP-1, RIMS-1 and PKHD-1 or comprises a gene selected from the group consisting of IFN-λ3, SULF-2, WWOX-1, RTFN-1, CACNA2D3, CASP-1, RIMS-1 and PKHD-1 or is encoded by a gene selected from the group consisting of IFN-λ3, SULF-2, WWOX-1, RTFN-1, CACNA2D3, CASP-1, RIMS-1 and PKHD-1.

Alternatively, or in addition, at least one marker comprises a polymorphic nucleotide in a sequence selected from the group consisting of:

(i) a sequence set forth in any one of SEQ ID NOs: 1 to 60, 62, 64 to 67, 69, 71 to 74, 76, 78, 79, 81 or 83 to 158; and
(ii) a sequence complementary to a sequence at (i).

Alternatively, or in addition, at least one marker comprises an allele associated with a positive response or a high response or a strong response to treatment with the immunomodulatory composition, wherein said allele is contained within a sequence selected from the group consisting of:

(i) a sequence set forth in any one of SEQ ID NOs: 5, 10, 67, 85, 88, 91, 94, 97, 100, 103, 106, 109, 112, 115, 118, 121, 124, 127, 130, 133, 136, 139, 142, 145, 148, 151, 154 and 157; and
(ii) a sequence complementary to a sequence at (i),
wherein detection of said at least one marker is indicative of a response of the subject to treatment with said composition.

Alternatively, or in addition, at least one marker comprises an allele associated with a low response or non-response to treatment with the immunomodulatory composition, wherein said allele is contained within a sequence selected from the group consisting of:

(i) a sequence set forth in any one of SEQ ID NOs: 6, 11, 69, 86, 89, 92, 95, 98, 101, 104, 107, 110, 113, 116, 119, 122, 125, 128, 131, 134, 137, 140, 143, 146, 149, 152, 155 and 158; and
(ii) a sequence complementary to a sequence at (i),
wherein detection of said at least one marker is indicative of a low response or non-response to treatment of the subject to treatment with said composition.

In a particular example, at least one marker is linked to an IFN-λ3 gene or is contained within an IFN-λ3 gene or comprises an IFN-λ3 gene or is encoded by an IFN-λ3 gene. In accordance with this example, at least one marker comprises a polymorphic nucleotide in a sequence selected from the group consisting of: (i) a sequence set forth in any one of SEQ ID NOs: 1 to 60, 62, 64 to 67, 69, 71 to 74, 76, 78, 79, 81 or 83 to 89; and (ii) a sequence complementary to a sequence at (i). For identifying a positive response using markers associated with the IFN-λ3 gene, at least one marker may comprise an allele associated with a response to treatment with the immunomodulatory composition, wherein said allele is contained within a sequence selected from the group consisting of: (i) a sequence set forth in any one of SEQ ID NOs: 5, 10, 67, 85 and 88; and (ii) a sequence complementary to a sequence at (i), wherein detection of said at least one marker is indicative of a response of the subject to treatment with said composition. Alternatively, to identify non-responders or weak responders, at least one marker may comprise an allele associated with a low response or non-response to treatment with the immunomodulatory composition, wherein said allele is contained within a sequence selected from the group consisting of: (i) a sequence set forth in any one of SEQ ID NOs: 6, 11, 69, 86 and 89; and (ii) a sequence complementary to a sequence at (i), wherein detection of said at least one marker is indicative of a low response or non-response to treatment of the subject to treatment with said composition.

Alternatively, or in addition, at least one proteinaceous marker is encoded by a sequence comprising a polymorphic nucleotide, wherein said sequence is selected from the group consisting of: SEQ ID NOs: 60, 62, 67, 69, 74, 76, 79 and 81, e.g., a marker comprising an amino acid sequence comprising a polymorphic amino acid, wherein said sequence is selected from the group consisting of: SEQ ID NOs: 61, 63, 68, 70, 75, 77, 80 and 82. Of these markers, an exemplary responder allele or high response allele i.e., an allele associated with a response to treatment with the immunomodulatory composition, is encoded by a sequence comprising a polymorphic nucleotide in SEQ ID NO: 67 or comprises the sequence of SEQ ID NO: 68. Alternatively, an exemplary non-responder allele or low response allele i.e., an allele associated with non-response or a poor response to treatment with the immunomodulatory composition, is encoded by a sequence comprising a polymorphic nucleotide in SEQ ID NO: 69 or comprises the sequence of SEQ ID NO: 70.

It is clearly within the scope of the invention to detect a plurality of the markers described according to any example hereof e.g., two or three or four of five or six or more of the markers.

It is also clearly within the scope of the invention to detect a haplotype comprising a plurality of the markers e.g., wherein the haplotype comprises an allele at rs8099917 such as wherein the haplotype comprises an allele at each of rs12980275, rs8105790, rs8103142, rs10853727, rs8109886 and rs8099917, and wherein detection of a haplotype comprising said allele is indicative of a low response or non-response to treatment of the subject to treatment with said composition. For example, an allele comprising a C or G nucleotide at rs8099917 is indicative of a low response or non-response to treatment of the subject to treatment with said composition. Alternatively, a haplotype comprising an allele at each of rs12980275, rs8105790, rs8103142, rs10853727, rs8109886 and rs8099917 may be indicative of a response to treatment of the subject to treatment with said composition.

The present invention also encompasses the detection of a modified level of expression e.g., increased or reduced expression of one or more of genes in a sample from the subject, wherein said modified expression is indicative of a response of the subject to treatment with said composition. Alternatively, modified expression e.g., increased or reduced expression of one or more of the genes, wherein said modified expression may be indicative of a low response or non-response to treatment. To detect modified expression, a modified level of at least one expression product of the gene(s) is detected e.g., by nucleic acid-based assay or antigen-based assay. For example, an amplification reaction, e.g., isothermal amplification or PCR reaction such as RT-PCR, is performed to detect an mRNA transcript of the gene(s) in a sample from the subject. Alternatively, to detect expressed protein, a protein-containing sample derived from a subject is contacted with an antibody or ligand capable of specifically binding to an allelic variant of a protein encoded by the gene(s) said marker for a time and under conditions sufficient for complex to form and the complex is detected. Any standard immunoassay may be employed e.g., ELISA, including sandwich ELISA performed in a microtiter well or in a lateral flow or flow-through assay format. In any assay to determine expression, it is possible to control for variability e.g., by comparing expression in the sample to expression in a control sample. Preferred control samples are selected from the group consisting of: (i) sample(s) from one or more subjects not being treated with the immunomodulatory composition; and (ii) a data set comprising measurements of expression determined previously for the sample(s) at (i).

In performing the prognostic method of the invention, or any diagnostic or therapeutic assay or process employing the method, the sample will generally comprise genomic DNA, mRNA, protein or a derivative thereof. Amplified DNA or cDNA derived from genomic DNA or mRNA is also useful. Accordingly, a nucleated cell and/or an extract thereof comprising protein or nucleic acid, is particularly useful if the assay is nucleic acid-based or protein-based. For protein-based assays e.g., immunoassay, the sample should comprise cell extract expected to comprise the marker protein e.g., a cell expressing IFN-λ3. Accordingly, the present invention encompasses the use of any sample selected from the group consisting of whole blood, serum, plasma, peripheral blood mononuclear cells (PBMC), a buffy coat fraction, saliva, urine, a buccal cell, liver biopsy and a skin cell or combinations thereof.

It is to be understood that the invention may be performed ex vivo i.e., wherein the sample has been derived or isolated or obtained previously from the subject.

The sample may comprise genomic DNA, mRNA, protein or a derived thereof.

In accordance with the prognostic method of the invention as described according to any example hereof, a positive response may be selected from the group consisting of: (i) a response comprising enhanced clearance of a virus or a reduction in virus titer or a change in other health characteristic of the subject related to reduced virus titer or enhanced clearance; (ii) a response comprising recovery or remission from cancer or reduced growth of a tumor or pre-cancerous lesion; (iii) a change in Th1 cell number, Th2 cell number or Th1/Th2 cell balance or a change in other health characteristic of the subject indicative of recovery from a Th1-mediated or Th2-mediated disease; and (iv) a combination of two or all of (i) to (iii). Similarly, a low response or non-response may be selected from the group consisting of: (i) a failure to clear of a virus or to reduce virus titer or change in other health characteristic of the subject related to said failure; (ii) a failure to recover or enter remission from cancer or to reduce growth of a tumor or pre-cancerous lesion; (iii) no significant change in Th1 cell number, Th2 cell number or Th1/Th2 cell balance or health characteristic of the subject that would indicate recovery from a Th1-mediated or Th2-mediated disease; and (iv) a combination of two or all of (i) to (iii).

For those diseases and conditions in which racial origin or genetic background is significant in the association with response, it is preferred that the subject belongs to that racial background or has a matching genetic background. In one example, the subject is Caucasian e.g., northern European. Alternatively, the subject may be African e.g., Zulu, or Asian e.g., Chinese.

The immunomodulatory composition may also comprise one or more IFNs and/or one or more derivatives of said one or more of said IFNs e.g., one or more IFNs selected from IFN-α, IFN-β, IFN-ω, IFN-γ, IFN-λ1, IFN-λ2 and IFN-λ3 and/or one or more derivatives of any one or more of said IFNs. Alternatively, or in addition the immunomodulatory composition may comprise one or more guanosine analogs and/or one or more derivatives of said one or more of said guanosine analogs e.g., one or more of ribavirin, viramidine, 7-benzyl-8-bromoguanine, 9-benzyl-8-bromoguanine, and CpG-containing oligonucleotide(s), and derivative(s), salt(s), solvate(s) and hydrate(s) thereof. For example, the immunomodulatory composition comprises IFN-α and ribavirin. Testing of responses to pegylated IFNs are clearly encompassed.

In another example, the present invention provides a process for accurately determining the likelihood that a subject will respond to treatment of Th1-mediated disease and/or Th2-mediated disease with an immunomodulatory composition, said process comprising performing the method as described according to any example hereof to thereby detect one or more markers indicative of the likely response of the subject to treatment with said composition, and determining a response for the subject selected from the group consisting of:

(i) a change in Th1 cell number, Th2 cell number or Th1/Th2 cell balance or a change in other health characteristic of the subject indicative of recovery from a Th1-mediated or Th2-mediated disease, wherein said response is indicative of a response to treatment; and
(ii) no significant change in Th1 cell number, Th2 cell number or Th1/Th2 cell balance or health characteristic of the subject that would indicate recovery from a Th1-mediated or Th2-mediated disease, wherein said response is indicative of a low response or no response to treatment.

In accordance with this example, the disease may be selected from the group consisting of multiple sclerosis (MS), rheumatoid arthritis (RA), Type I diabetes (IDDM), scleroderma, Con A hepatitis, atopic dermatitis, asthma, allergic rhinitis and allergy.

Alternatively, another example of the present invention provides a process for accurately determining the likelihood that a subject will respond to treatment of one or more bacterial or viral infections with an immunomodulatory composition, said process comprising performing a method as described according to any example hereof to thereby detect one or more markers indicative of the likely response of the subject to treatment with said composition, and determining a response for the subject selected from the group consisting of:

(i) a response comprising enhanced clearance of a virus or bacterium or a reduction in virus titer or bacterial count or a change in other health characteristic of the subject related to reduced virus titer or bacterial count or enhanced clearance, wherein said response is indicative of a response to treatment; and
(ii) a failure to clear of a virus or bacteria or to reduce virus titer or bacterial count or a change in a health characteristic of the subject related to said failure, wherein said response is indicative of a low response or no response to treatment.

In accordance with this example, the bacterium is a gram negative bacterium and/or the virus is a single-stranded RNA virus e.g., a virus is selected from the group consisting of a human papillomavirus, apicornavirus, a flavivirus such as a hepatitis virus, an arenavirus, a togavirus, a bunyavirus, a filovirus, a paramyxovirus, a rhabdovirus, an orthomyxovirus, and a coronavirus. Alternatively, the virus is a DNA virus e.g., a herpesvirus.

Alternatively, another example of the present invention provides a process for accurately determining the likelihood that a subject will respond to treatment of one or more neoplasia or pre-cancerous conditions with an immunomodulatory composition, said process comprising performing a method of the invention according to any example hereof to thereby detect one or more markers indicative of the likely response of the subject to treatment with said composition, and determining a response for the subject selected from the group consisting of:

(i) a response comprising recovery or remission from cancer or reduced growth of a tumor or pre-cancerous lesion, wherein said response is indicative of a response to treatment; and
(ii) a failure to recover or enter remission from cancer or to reduce growth of a tumor or pre-cancerous lesion, wherein said response is indicative of a low response or no response to treatment.

In accordance with this example, the cancer or pre-cancerous lesion is selected from the group consisting of breast cancer, lung cancer, ovarian cancer, HPV-associated cancer (e.g., cervical intraepithelial neoplasia and/or cervical carcinoma and/or vulvar intraepithelial neoplasia and/or penile intraepithelial neoplasia and/or perianal intraepithelial neoplasia), hepatocellular carcinoma, basal cell carcinoma, squamous cell carcinoma, actinic keratosis, melanoma, hairy cell leukemia, Kaposi's sarcoma, non-Hodgkin's lymphoma, astrocytoma, glioblastoma, thymoma, adenocarcinoma and fibrosarcoma.

Yet another example of the invention provides a process for accurately determining the likelihood that a subject will respond to treatment of HCV infection with an immunomodulatory composition, said process comprising performing a method of the invention according to any example hereof to thereby detect one or more markers indicative of the likely response of the subject to treatment with said composition, and determining a response for the subject selected from the group consisting of:

(i) a response comprising enhanced clearance of HCV or a reduction in HCV titer or a change in other health characteristic of the subject related to reduced virus titer or enhanced clearance, wherein said response is indicative of a response to treatment; and
(ii) a failure to clear HCV or to reduce HCV titer or a change in a health characteristic of the subject related to said failure, wherein said response is indicative of a low response or no response to treatment.

In yet another example, the present invention provides a process for accurately determining the likelihood that a subject will respond to treatment of HCV infection with an immunomodulatory composition comprising an IFN or a derivative thereof and ribavirin or a derivative thereof, said process comprising performing a method according to any example hereof to thereby detect one or more markers indicative of the likely response of the subject to treatment with said composition, and determining a response for the subject selected from the group consisting of:

(i) a response comprising enhanced clearance of HCV or a reduction in HCV titer or a change in other health characteristic of the subject related to reduced virus titer or enhanced clearance, wherein said response is indicative of a response to treatment; and
(ii) a failure to clear HCV or to reduce HCV titer or a change in a health characteristic of the subject related to said failure, wherein said response is indicative of a low response or no response to treatment.

In these foregoing examples, the immunomodulatory composition may comprise one or more IFNs and/or one or more derivatives of said one or more of said IFNs as described according to any other example hereof. Alternatively, or in addition, the immunomodulatory composition may comprise one or more guanosine analogs and/or one or more derivatives of said one or more of said guanosine analogs according to any other example hereof.

In another example, the present invention provides a process for selecting a subject in need of treatment with an immunomodulatory composition, said process comprising:

(i) exposing a sample comprising cells obtained from the subject to the immunomodulatory composition in vitro; and
(ii) performing a prognostic method or process as described according to any example hereof on the sample to thereby identify a subject likely to respond to treatment with the immunomodulatory composition; and
(iii) administering or recommending an immunomodulatory composition to a subject likely to respond to treatment.

In another example, the present invention provides a process for selecting a subject in need of treatment with an immunomodulatory composition, said process comprising:

(i) exposing a sample comprising cells obtained from the subject to the immunomodulatory composition in vitro; and
(ii) performing a prognostic method or process as described according to any example hereof on the sample to thereby identify a subject likely to not respond to treatment with the immunomodulatory composition or likely to provide a low response to treatment; and
(iii) administering or recommending an alternative therapy to the immunomodulatory composition.

This selection process is readily-performed on a sample from a subject that has not been previously administered with the immunomodulatory composition, or for determining whether or not to continue treatment in a subject who has received prior in vivo administration of the immunomodulatory composition. This method is particularly well-suited to determining the effect of an immunomodulatory composition on a sample from a subject infected with HCV. In this example, the immunomodulatory composition may comprise one or more IFNs and/or one or more derivatives of said one or more of said IFNs as described according to any other example hereof. Alternatively, or in addition, the immunomodulatory composition may comprise one or more guanosine analogs and/or one or more derivatives of said one or more of said guanosine analogs according to any other example hereof. Exemplary samples comprise peripheral blood mononuclear cells.

In a related example, the present invention provides a process for treating an HCV-infected subject, comprising performing the ex vivo selection process on a sample from a subject and administering or recommending a therapeutically effective amount of an immunomodulatory composition comprising an IFN to the subject if the subject is likely to respond to treatment or administering or recommending an alternative therapy if the subject is not likely to respond to treatment or likely to produce a low response to treatment.

In another example, the present invention provides a process for determining a predisposition in a subject to a chronic HCV infection, said process comprising performing a prognostic method as described herein to thereby identify a subject likely to not respond to treatment with an immunomodulatory composition or likely to provide a low response to treatment, and determining that the subject has a predisposition to chronic HCV infection.

In yet another example, the present invention provides methods of treatment employing the prognostic test described herein. For example, the invention provides a process comprising: (i) performing a prognostic method or process as described according to any example hereof; and (ii) administering or recommending an immunomodulatory composition to a subject. In another example, such a process comprises: (i) obtaining results of a prognostic method or process as described according to any example hereof; and (ii) administering or recommending an immunomodulatory composition to a subject.

In another example, the present invention provides a method of treatment of HCV-infection in a subject, said method comprising administering or recommending to the subject an immunomodulatory composition comprising an IFN-λ2 or a derivative thereof and/or an IFN-λ3 or a derivative thereof to a subject in need thereof e.g., wherein administration of the immunomodulatory composition is for a time and under conditions sufficient to enhance viral clearance or reduce virus titer in the subject. As will be known to the skilled artisan, a derivative may be pegylated and e.g., the invention clearly encompasses administration of pegylated IFN-λ2 and/or pegylated IFN-λ3. Alternatively, or in addition, the derivative may be modified by addition of albumin i.e., it is “albuminated”, and e.g., the invention clearly encompasses administration of albuminated IFN-λ2 and/or albuminated IFN-λ3. Optionally, a guanosine analog as described according to any example hereof may also be administered to the subject.

A further example of the invention provides for a use of IFN-λ2 and/or IFN-λ3 is used in the preparation of a medicament for the treatment of a medical condition known to be treated using IFN-α/β. Such medical indications are apparent from the disclosure herein.

A further example of the invention provides for a use of IFN-λ2 is used in the preparation of a medicament for the treatment of infection by HCV.

A further example of the invention provides for a use of IFN-λ3 is used in the preparation of a medicament for the treatment of infection by HCV.

A further example of the invention provides for a use of an IFN is used in the preparation of a medicament for the treatment of infection by a gram-negative bacterium.

A further example of the invention provides for a use of an IFN is used in the preparation of a medicament for the treatment of polycystic kidney disease or complication arising there from e.g., post-transplant diabetes.

A further example of the invention provides for a use of an IFN is used in the preparation of a medicament for the treatment of a carcinoma of the lung, ovary, liver or breast.

A further example of the present invention provides a kit comprising a plurality of isolated nucleic acids and/or a plurality of antibodies and/or a plurality of peptides for performing a prognostic method or process according to any example hereof. In one example, the nucleic acids each comprise an allele of a SNP listed in Table 1 and are capable of distinguishing between the other allele at the same locus e.g., by virtue of comprising nucleotide sequences set forth herein or complementary thereto, or by virtue of being contained within said nucleotide sequences. In another example, the antibodies bind to a peptide comprising an allelic variant of an amino acid in the IFN-λ3 polypeptide as set forth in Table 1 and are capable of distinguishing between the other allelic variant at the same locus. In another example, the peptides each comprise an allelic variant of an amino acid in the IFN-λ3 polypeptide as set forth in Table 1 and are capable of distinguishing between the other allelic variant at the same locus e.g., by virtue of comprising amino acid sequences set forth herein, or by virtue of being contained within said amino acid sequences. The plurality of nucleic acids, peptides or antibodies may be arrayed e.g., on a solid substrate. Preferably, the kit at least comprises a plurality of nucleic acids comprising sequences derived from the IFN-λ3 gene or at least comprises a plurality of peptides derived from the full-length sequence of the IFN-λ3 polypeptide, or at least comprise a plurality of antibodies each capable of binding to a peptide derived from the full-length sequence of the IFN-λ3 polypeptide. The plurality of nucleic acids, peptides or antibodies may be arrayed e.g., on a solid substrate. A further example provides for the use of a plurality of isolated nucleic acid or peptides or antibodies as described according to any example hereof in the manufacture of a kit or solid substrate for performing a prognostic method or process according to any example hereof.

### 3. General

Unless the context requires otherwise or specifically stated to the contrary, integers, steps, or elements of the invention recited herein as singular integers, steps or elements clearly encompass both singular and plural forms of the recited integers, steps or elements.

The designation of nucleotide residues referred to herein are those recommended by the IUPAC-IUB Biochemical Nomenclature Commission, wherein A represents Adenine, C represents Cytosine, G represents Guanine, T represents Thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, developmental biology, mammalian cell culture, recombinant DNA technology, histochemistry and immunohistochemistry and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;

### BRIEF DESCRIPTION OF THE DRAWINGS

FIG. 1 provides a graphical representation showing combined expression of IFN-λ2 and IFN-λ3 (y-axis) as determined by RT-PCR for patients having different genotypes at rs8099917 (x-axis). Data show that expression of IFN-λ2 and IFN-λ3 is reduced in patients that are homozygous for the low response (LR) G allele at this locus compared to those patients that are homozygous for the high response (HR) T allele at this locus, and intermediate for G/T heterozygotes. The data further suggest functional significance of the rs8099917 SNP in therapeutic response.

### DETAILED DESCRIPTION OF THE PREFERRED EMBODIMENTS

### Markers Associated with a Disease or Disorder

In one example, a marker of the present invention is presented in Table 1 and preferably in Tables 3 or 4-5.

Preferably, the marker comprises or consists of nucleic acid comprising a sequence set forth in the Sequence Listing or complementary thereto. Such a nucleic acid marker comprises, for example, a polymorphism, an insertion into an IFN-LAMBDA3 gene or transcript thereof, a deletion from an IFN-LAMBDA3 gene or transcript thereof, a transcript of an IFN-LAMBDA3 gene or a fragment thereof or an alternatively spliced transcript of an IFN-LAMBDA3 or a fragment thereof, and includes copy number variants or inversions. The nucleotide substitution or deletion or insertion may be in the 5′-end of a gene, the 3′-end of a gene, in an exon of a gene or an intron of a gene. Alternatively, the nucleotide substitution or deletion or insertion may be in an intergenic region i.e., between genes. A nucleotide substitution or deletion or insertion may modify gene expression and, without being bound by any theory or mode of action this modified expression may be associated with the development of a therapeutic response, or a non-response or low response.

Markers comprising proteins or peptides spanning a prognostic polymorphism are also provided by this invention.

In one example, the method of the invention comprises detecting or determining the presence of a plurality of markers associated with a therapeutic response.

TABLE 1
Summary of SNPs associated with response to therapy

| SNP | Chromosome | Position¹ | Location² | SNP effect | Sequence comprising SNP | SEQ ID NO: |
|---|---|---|---|---|---|---|
| rs4803224 | 19 | 44444854 | IL28A/IL28B intergenic | expressifon level | aaaaaaaaatagaagaattatctgggcatg[C/G]tggtgggtgcctgcagctcc | 1 |
| | | | region | | agctgcttag | |
| rs12980602 | 19 | 44444660 | IL28A/IL28B intergenic | expression level | atattcatataacaatatgaaagccagaga[C/T]agctcgtctgagacacagat | 2 |
| | | | region | | gaacaaaaac | |
| rs10853728 | 19 | 44436986 | IL28A/IL28B intergenic | Weak | tgtctcgtaagcagcctgggagatgtgggc[C/G]taagctttggtgaggatgag | 3 |
| | | | region | | agtctgtctt | |
| rs8099917 | 19 | 44435005 | 5′-end of IL28B | expression level | cctccttttgttttcctttctgtgagcaat[G/T]tcacccaaattggaaccatg | 4 |
| | | | | | ctgtatacag | |
| | | | | HR allele = T | cctccttttgttttcctttctgtgagcaatTtcacccaaattggaaccatgctgt | 5 |
| | | | | | atacag | |
| | | | | LR allele = G | cctccttttgttttcctttctgtgagcaatGtcacccaaattggaaccatgctgt | 6 |
| | | | | | atacag | |
| rs8113007 | 19 | 44434943 | 5′-end of IL28B | expression level | ttaaagtaagtcttgtatttcacctcctgg[A/T]ggtaaatattttttaacaat | 7 |
| | | | | | ttgtcactgt | |
| rs8109889 | 19 | 44434610 | 5′-end of IL28B | expression level | catttttccaacaagcatcctgccccaggt[C/T]gctctgtctgtctcaatcaa | 8 |
| | | | | | tctctttttg | |
| rs8109886 | 19 | 44434603 | 5′-end of IL28B | expression level | ttcttattcatttttccaacaagcatcctg[A/C]cccaggtcgctctgtctgtc | 9 |
| | | | | | tcaatcaatc | |
| | | | | HR allele = C | ttcttattcatttttccaacaagcatcctgCcccaggtcgctctgtctgtctcaa | 10 |
| | | | | | tcaatc | |
| | | | | LR allele = A | ttcttattcatttttccaacaagcatcctgAcccaggtcgctctgtctgtctcaa | 11 |
| | | | | | tcaatc | |
| rs61599059 | 19 | 44434538 | 5′-end of IL28B | expression level | gtcttgctttctctttctctctctctctct[*/CT]gttcctgtctctgtctctg | 12, 13 |
| | | | | | gcgtgactcca | |
| rs34567744 | 19 | 44434535 | 5′-end of IL28B | expression level | tgtgtcttgctttctctttctctctctctc[*/CT]tctgttcctgtctctgtct | 14, 15 |
| | | | | | ctggcgtgact | |
| rs10642510 | 19 | 44434534 | 5′-end of IL28B | expression level | tgtcttgctttctctttctctctctct[*/CT/TC]ctctgttcctgtctctgtc | 16, 17, 18 |
| | | | | | tctggcgt | |
| rs 10643535 | 19 | 44434531 | 5′-end of IL28B | expression level | tcctgtgtcttgctttctctttctctctct[**/CT]ctctctgttcctgtctct | 19, 20 |
| | | | | | gtctctggcgtg | |
| rs34593676 | 19 | 44434523 | 5′-end of IL28B | expression level | agcgtctcctcctgtgtcttgctttctctt[**/TC]tctctctctctctctgtt | 21, 22 |
| | | | | | cctgtctctgtc | |
| rs 25122122 | 19 | 44434521 | 5′-end of IL28B | expression level | tcagcgtctcctcctgtgtcttgctttctc[*/T]tttctctctctctctctgtt | 23, 24 |
| | | | | | cctgtctctg | |
| rs35407108 | 19 | 44434307 | 5′-end of IL28B | expression level | gcctgggcaacaaaagtgaaactccgtctc[*/A]aaaaaaaaaaaagacacaaa | 25, 26 |
| | | | | | agggaggttc | |
| rs59211796 | 19 | 44434282 | 5′-end of IL28B | expression level | gccgagatcacgccattgcactccagcctg[A/G]gcaacaaaagtgaaactccg | 27 |
| | | | | | tctcaaaaaa | |
| rs62120529 | 19 | 44434043 | 5′-end of IL28B | expression level | aaaaaagacacaaaccaggcacagtcgctc[A/G]tgcctgtaatcccagcactt | 28 |
| | | | | | tgggaggccg | |
| rs62120528 | 19 | 44433258 | 5′-end of IL28B | expression level | cttgaggtcaggagttcaataccagcctga[A/C]caacatggcaaaaccctgtc | 29 |
| | | | | | tctactagaa | |
| rs12983038 | 19 | 44432964 | 5′-end of IL28B | expression level | ggagggaggattgtttgagcccaggagttc[A/G]agaccagcctgggcaatata | 30 |
| | | | | | gtgagaccct | |
| rs10853727 | 19 | 44432303 | 5′-end of IL28B | weak | tttgctgaacatacatcatatgaagaggca[C/T]gcttatgatctgcacctgcg | 31 |
| | | | | | tctggagttg | |
| rs7254424 | 19 | 44432022 | 5′-end of IL28B | expression level | aattcttggattacaggcatgatccattgc[A/G]cctggcctcattattttctt | 32 |
| | | | | | aaaccgtttt | |
| rs1549928 | 19 | 44431549 | 5′-end of IL28B | expression level | gaagcaaagaaagaggaaacagacagtaga[A/G]acagggacagagacaatttg | 33 |
| | | | | | gaaaccgagt | |
| rs34347451 | 19 | 44431529 | 5′-end of IL28B | expression level | gggatggctgccctccaacactcggtttcc[*/A]aaattgtctctgtccctgtt | 24, 35 |
| | | | | | tctactgtct | |
| rs35814928 | 19 | 44431477 | 5′-end of IL28B | expression level | tctgggatcccagtcgggtgtgaggacttc[*/A]aacccgaggttggcctgtgc | 36, 37 |
| | | | | | ccgggatggc | |
| rs4803222 | 19 | 44431193 | 5′-end of IL28B | expression level | gagcgtgaaggcacagcacacacagtggga[C/G]agagagtgggagccggcccc | 38 |
| | | | | | ctcctcgcct | |
| rs11322783 | 19 | 44430995 | 5′-end of IL28B | expression level | agtgcgagagcaggcagcgccggggggcct[*/T]ctgcgatcaccgtgcacagg | 39, 40 |
| | | | | | acccacagcc | |
| rs4803221 | 19 | 44430969 | 5′-end of IL28B | expression level | cagcgtccggggctccagcgagcggtagtg[C/G]gagagcaggcagcgccgggg | 41 |
| | | | | | ggccttctgc | |
| rs12979860 | 19 | 44430627 | 5′-end of IL28B | expression level | tgtactgaaccagggagctccccgaaggcg[C/T]gaaccagggttgaattgcac | 42 |
| | | | | | tccgcgctcc | |
| rs12971396 | 19 | 44429706 | 5′-end of IL28B | expression level | gaagaccacgctggctttgcggcaccgagg[C/G]gagtcctggagccagggagg | 43 |
| | | | | | gagggcagcg | |
| rs11672932 | 19 | 44429556 | 5′-end of IL28B | expression level | tcgcccggccagcccaatggacgacag[C/G]agctgctttcggcagccaatggc | 44 |
| | | | | | gtgg | |
| rs11882871 | 19 | 44429451 | 5′-end of IL28B | expression level | tccctgtagaaggacccgctcctctt[A/G]tatctgagacagtggatccaagtc | 45 |
| | | | | | ag | |
| rs56215543 | 19 | 44429428 | 5′-end of IL28B | expression level | gatataagaggagcgggtccttctac[A/G]gggaagagaccacagttctccagg | 46 |
| | | | | | aa | |
| rs12979731 | 19 | 44429353 | 5′-end of IL28B | expression level | tccagagctcaagttttttcctgcca[C/T]agcaaccgttggagggtcgtacaa | 47 |
| | | | | | tg | |
| rs2020358 | 19 | 44428927 | 5′-end of IL28B | expression level | cgagccagggactcaggtggcctgag[G/T]ttcagttctgaccctgccagttaa | 48 |
| | | | | | tt | |
| rs34853289 | 19 | 44428781 | 5′-end of IL28B | expression level | tcattaagaccatactaggacctcag[C/T]tggagagtttaaaacgtgatctca | 49 |
| | | | | | ac | |
| rs8107030 | 19 | 44428559 | 5′-end of IL28B | expression level | gggtgccgtctttcttagggaagttc[A/G]ggcagtggtgaagagcatgggtct | 50 |
| | | | | | tg | |
| rs41537748 | 19 | 44428498 | 5′-end of IL28B | expression level | aggctctgctcaaga[C/T]tgaggtgtgacgaagg | 51 |
| rs59702201 | 19 | 44428148 | 5′-end of IL28B | expression level | gcatatatatatatatatatatatat[*/ATAT]tttgagacagggtcttgttcg | 52, 53 |
| | | | | | gtcac | |
| rs2596806 | 19 | 44428010 | 5′-end of IL28B | expression level | taagacagggtctcactctgtcactg[C/G]agtgcaatggcatgatcacagctc | 54 |
| | | | | | ac | |
| rs2569377 | 19 | 44427950 | 5′-end of IL28B | expression level | gtaacctacaggaaggtatgttccca[A/G]gaggattccacctgctctggtttt | 55 |
| | | | | | gt | |
| rs4803219 | 19 | 44427759 | 5′-end of IL28B | expression level | ctgagctccatggggcagcttttatc[C/T]ctgacagaagggcagtcccagctg | 56 |
| | | | | | at | |
| rs28416813 | 19 | 44427484 | 5′-end/intron 1 of IL28B | expression | cagagagaaagggagctgagggaatg[C/G]agaggctgcccactgagggcaggg | 57 |
| | | | | level/mRNA | gc | |
| | | | | stability/turnover/ | | |
| | | | | alternate splicing | | |
| rs630388 | 19 | 44427442 | exon 1 of IL28B | silent mutation in | agcaccagcactggcatgcagtcccc[A/G]gtcatgtctgtgtcacagagagaa | 58 |
| | | | | codon for Thr6 of | ag | |
| | | | | IL28B | | |
| rs629976 | 19 | 44427365 | exon 1 of IL28B | missense: R32H | tggagcagttcctgtcgccaggctcc[A/G]cggggctctcccggatgcaagggg | 59 |
| | | | | mutation in IL28B | ct | |
| | | | | IL28B-His32 allele | tggagcagttcctgtcgccaggctccAcggggctctcccggatgcaaggggct | 60 |
| | | | | IL28B-Arg32 allele | tggagcagttcctgtcgccaggctccGcggggctctcccggatgcaaggggct | 62 |
| rs629008 | 19 | 44427130 | intron 2 of IL28B | mRNA | cttcaggaaaacatgagtcagtccct[A/G]cagtaggagcatgagatagcccac | 64 |
| | | | | stability/turnover/ | tg | |
| | | | | alternate splicing | | |
| rs628973 | 19 | 44427106 | intron 2 of IL28B | mRNA | gggaggatggtagaggaccctcttck[A/T]maggaaaacatgagtcagtccctg | 65 |
| | | | | stability/turnover/ | ca | |
| | | | | alternate splicing | | |
| rs8103142 | 19 | 44426946 | exon 2 of IL28B | missense: K74R | tcctggggaagaggcgggagcggcac[C/T]tgcagtccttcagcagaagcgact | 66 |
| | | | | mutation in IL28B | ct | |
| | | | | HR allele = T or A | agagtcgcttctgctgaaggactgcaAgtgccgctcccgcctcttccccagga | 67 |
| | | | | (IL28B-Lvs74) | | |
| | | | | LR allele = C or G | agagtcgcttctgctgaaggactgcaGgtgccgctcccgcctcttccccagga | 69 |
| | | | | (IL28B-Arg74) | | |
| rs8102358 | 19 | 44426852 | intron 3 of IL28B | mRNA | gtgaaggggccactacagagccaggt[A/G]agcagggctgggagggcaggggtg | 71 |
| | | | | stability/turnover/ | gg | |
| | | | | alternate splicing | | |
| rs11881222 | 19 | 44426763 | intron 3 of IL28B | mRNA | agagggcacagccagtgtggtcaggt[A/G]ggagcagagggaaggggtagcagg | 72 |
| | | | | stability/turnover/ | tg | |
| | | | | alternate splicing | | |
| rs61735713 | 19 | 44426330 | exon 4 of IL28B | missense: H160Y | cccggggccgcctccaccattggctg[C/T]accggctccaggaggccccaaaaa | 73 |
| | | | | mutation in IL28B | ag | |
| | | | | IL28B-His160 | cccggggccgcctccaccattggctgCaccggctccaggaggccccaaaaaag | 74 |
| | | | | IL28B-Tyr160 | cccggggccgcctccaccattggctgTaccggctccaggaggccccaaaaaag | 76 |
| rs62120527 | 19 | 44426192 | exon 5 of IL28B | missense: E175K | gaagaggttgaaggtgacagaggcct[C/T]gaggcagccaggggactcctgtag | 78 |
| | | | | mutation in IL28B | gg | |
| | | | | IL28B-Glu175 | ccctacaggagtcccctggctgcctcGaggcctctgtcaccttcaacctcttc | 79 |
| | | | | IL28B-Lys175 | ccctacaggagtcccctggctgcctcAaggcctctgtcaccttcaacctcttc | 81 |
| rs4803217 | 19 | 44426060 | 3′-end of IL28B | mRNA | Tagcgactgggtgacaataaattaag[A/C]caagtggctaatttataaataaaa | 83 |
| | | | | stability/turnover | t | |
| rs8105790 | 19 | 44424341 | 1.75 kb distal to 3′-end | mRNA | ttcccttcctgacatcactccaatgtcctg[C/T]ttctgtggttacatcttccg | 84 |
| | | | of IL28B | stability/turnover | ctaatgatgc | |
| | | | | HR allele = T | ttcccttcctgacatcactccaatgtcctgTttctgtggttacatcttccgctaa | 85 |
| | | | | | tgatgc | |
| | | | | LR allele = C | ttcccttcctgacatcactccaatgtcctgCttctgtggttacatcttccgctaa | 86 |
| | | | | | tgatgc | |
| rs12980275 | 19 | 44423623 | 2.47 kb distal to 3′-end | mRNA | Ctgagagaagtcaaattcctagaaac[A/G]gacgtgtctaaatatttgccgggg | 87 |
| | | | of IL28B | stability/turnover | t | |
| | | | | HR allele = A | ctgagagaagtcaaattcctagaaacAgacgtgtctaaatatttgccggggt | 88 |
| | | | | LR allele = G | ctgagagaagtcaaattcctagaaacGgacgtgtctaaatatttgccggggt | 89 |
| rs7750468 | 6 | 118183677 | Intergenic to C6orf68 | HR allele = A | | |
| | | | and SLC35F1 | LR allele = G | taaatgaaatttggaaaacaatccag[A/G]aacaaaatgagaaaatagacaaag | 90, 91, 92 |
| | | | | | a | |
| rs2746200 | 6 | 73075162 | RIMS-1 gene intron | HR allele = C | | |
| | | | | LR allele = T | ggagggtcactgtgattcagtgatgc[C/T]caactccctaagagtcttaccaaa | 93, 94, 95 |
| | | | | | a | |
| rs927188 | 6 | 51917576 | PHKD-1 gene intron | HR allele = A | | |
| | | | | LR allele = C | ttgtagaaattgagcaggttgtagat[A/C]taatcacccggtgggttcttcctg | 96, 97, 98 |
| | | | | | c | |
| rs2517861 | 6 | 29929961 | Intergenic to HLA | HR allele = G | | |
| | | | pseudogenes HCP5P10 | LR allele = A | tgatatttcttcatgggatggtctcc[A/G]tgatacaatggtaagggaaaacag | 99, 100, 101 |
| | | | and MICF | | c | |
| rs2025503 | 6 | 23701746 | Intergenic to | HR allele = C | | |
| | | | ALDH5A1 and PRL | LR allele = A | catacactgtacaaagattttcactt[A/C]accaagttggaggactcacttgat | 102, 103, 104 |
| | | | | | c | |
| rs2066911 | 6 | 23656329 | Intergenic to | HR allele = C | | |
| | | | ALDH5A1 and PRL | LR allele = A | catacactgtacaaagattttcactt[A/C]accaagttggaggactcacttgat | 105. 106, 107 |
| | | | | | c | |
| rs10018218 | 4 | 161692769 | Intergenic region | HR allele = C | | |
| | | | | LR allele = T | atgggctcaaatctcatatccttcctccaa[C/T]acgtgttaaaactcaggccc | 108, 109, 110 |
| | | | | | tttggtgact | |
| rs1581096 | 4 | 44874493 | Intergenic region | HR allele = G | | |
| | | | | LR allele = A | aaaagagtacaagggatccattttccccat[A/G]tccttactaatacttgctat | 111, 112, 113 |
| | | | | | catttgtctt | |
| rs1250105 | 4 | 1193265 | Near to CTBP1 | HR allele = G | | |
| | | | | LR allele = A | aaaatcagccaaagcctgcagctaatcctg[A/G]gactggccaggtgacctcac | 114, 115, 116 |
| | | | | | aggagcgcct | |
| rs1939565 | 11 | 930139007 | Near to KIAA1731 and | HR allele = A | | |
| | | | intergenic to FN5 | LR allele = G | gcaaagcactggcactttattatatttacc | 117, 118, 119 |
| | | | | | [A/G]aaagtacttttggggagagaactaccctat | |
| rs568910 | 11 | 104409780 | Intron-2 of CASP-1 | HR allele = G | | |
| | | | | LR allele = T | ctgagtgcaaggggtctgtaggcacttatg[G/T]agttgtaaagtcacatgaag | 120, 121, 122 |
| | | | | | ctttaaggtt | |
| rs557905 | 11 | 104403053 | Intron-6 of CASP-1 | HR allele = G | | |
| | | | | LR allele = A | ccactttgggaatgcacatttagatatttc[A/G]tttccaaatcccaatcactc | 123, 124, 125 |
| | | | | | ccctctaccc | |
| rs6806020 | 3 | 54949198 | Intron of CACNA2D3 | HR allele = T | | |
| | | | | LR allele = C | aaaaaaccacacactcaccacattggtgtc[C/T]agtctcaggccacagcccca | 126, 127, 128 |
| | | | | | cactcccagt | |
| rs12486361 | 3 | 16430714 | Intron of RTFN-1 gene | HR allele = C | | |
| | | | | LR allele = T | aatagatagaagtgacaaaacctctgcctt[C/T]gtggagctaacaatctaata | 129, 130, 131 |
| | | | | | ggaggagaaa | |
| rs10283103 | 8 | 67556167 | intergenic ADHFE1 and | HR allele = C | | |
| | | | MGC33510 | LR allele = T | Agttctttattaataagtcacagcatcctg[C/T]aaggaagaaattgtgcatca | 132, 133, 134 |
| | | | | | gctgccaagc | |
| rs2114487 | 8 | 67420305 | Intergenic region RRS1 | HR allele = C | | |
| | | | and CRH | LR allele = T | aggacactggaaaagggatagaaacagatt[C/T]tcccccggggccttcagaac | 135, 136, 137 |
| | | | | | tgaaagtagt | |
| rs7196702 | 16 | 77341734 | Intron of WWOX gene | HR allele = A | | |
| | | | | LR allele = G | ttcatagctgtcttgcccctcctgtggtct[A/G]taagaatgggaccaggactc | 138, 139, 140 |
| | | | | | ctagttgtga | |
| rs3093390 | 16 | 27370949 | Near to IL21R and | HR allele = T | | |
| | | | intergenic to GFT3C1 | LR allele = C | gttgggaagagatatgcacaatctgccctc[C/T]tggctggtatgagtgagtcc | 141, 142, 143 |
| | | | | | cagctcaccg | |
| rs7512595 | 1 | 27729758 | Intergenic region | HR allele = G | | |
| | | | WASF2 and ADHC1 | LR allele = A | agaccaaatgcattaatacatatgcaaagc[A/G]tttggaacagctggcatata | 144, 145, 146 |
| | | | | | taagtgccat | |
| rs1002960 | 9 | 88029735 | Intergenic region | HR allele = A | | |
| | | | | LR allele = C | cggcccttgtctgcgtacccctagacttct[A/C]attatgtaagaaaaataacc | 147, 148, 149 |
| | | | | | actatttggt | |
| rs1931704 | 10 | 129229799 | Near to NPS and | HR allele = G | | |
| | | | intergenic to NPS and | LR allele = A | taggaggaaacgtgtgaagagggcttgggt[A/G]actctaagacagttacctca | 150, 151, 152 |
| | | | | | tgacaaagaa | |
| | | | DOCK1 | | | |
| rs66616 | 14 | 58286251 | Intergenic to DACT1 | HR allele = G | | |
| | | | and LOC729646 | LR allele = A | gaaaaacaagaaagctggtttctttgattt[A/G]acagacaatgtatagaccat | 153, 154, 155 |
| | | | | | ttgggcactg | |
| rs4402825 | 20 | 45765623 | Intron-3 of SULF2 gene | HR allele = T | | |
| | | | | LR allele = C | gtttgtggatcccttggattctgtctgcta[C/T]acagcaaccagaatggctaa | 156, 157, 158 |
| | | | | | cattaaagaa | |

¹Chromosome positions are derived from Hapmap project data release 27.
²Gene locations were obtained by scanning ±100 kb from the associated SNP
HR allele, Allele associated with higher response to therapy.
LR allele, Allele associated with lower response or no response to therapy.

### Assay Methods

### (i) Nucleic Acid Marker Detection

As will be apparent to the skilled artisan a probe or primer capable of specifically detecting a marker that is associated with or causative of a therapeutic response, is any probe or primer that is capable of specifically hybridizing to the region of the genome that comprises said marker, or an expression product thereof. Accordingly, a nucleic acid marker is preferably at least about 8 nucleotides in length (for example, for detection using a locked nucleic acid (LNA) probe). To provide more specific hybridization, a marker is preferably at least about 15 nucleotides in length or more preferably at least 20 to 30 nucleotides in length. Such markers are particularly amenable to detection by nucleic acid hybridization-based detection means assays, such as, for example any known format of PCR or ligase chain reaction.

In one example, a preferred probe or primer comprises, consists of or is within a nucleic acid comprising a nucleotide sequence at least about 80% identical to at least nucleotides of a sequence selected from the group consisting of:
(i) a sequence at least about 80% homologous to a sequence selected from the group consisting of SEQ ID NO: 1-158;
(ii) a sequence capable of encoding an amino acid sequence encoded by a sequence at (i) e.g., a sequence that is at least 80% homologous to the sequence set forth in SEQ ID NO: 68 or 70; and
(iii) a sequence complementary to a sequence set forth in (i) or (ii).

Generally, a method for detecting a nucleic acid marker comprises hybridizing an oligonucleotide to the marker linked to nucleic acid in a sample from a subject under moderate to high stringency conditions and detecting hybridization of the oligonucleotide using a detection means, such as for example, an amplification reaction or a hybridization reaction.

For the purposes of defining the level of stringency to be used in these diagnostic assays, a low stringency is defined herein as being a hybridization and/or a wash carried out in 6×SSC buffer, 0.1% (w/v) SDS at 28° C., or equivalent conditions. A moderate stringency is defined herein as being a hybridization and/or washing carried out in 2×SSC buffer, 0.1% (w/v) SDS at a temperature in the range 45° C. to 65° C., or equivalent conditions. A high stringency is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample DNA, and/or the type of hybridization probe used.

In another example, stringency is determined based upon the temperature at which a probe or primer dissociates from a target sequence (i.e., the probe or primers melting temperature or Tm). Such a temperature may be determined using, for example, an equation or by empirical means. Several methods for the determination of the Tm of a nucleic acid are known in the art. For example the Wallace Rule determines the G+C and the T+A concentrations in the oligonucleotide and uses this information to calculate a theoretical Tm (Wallace et al., Nucleic Acids Res. 6, 3543, 1979). Alternative methods, such as, for example, the nearest neighbour method are known in the art, and described, for example, in Howley, et al., J. Biol. Chem. 254, 4876, Santa Lucia, Proc. Natl. Acad. Sci. USA, 95: 1460-1465, 1995 or Bresslauer et al., Proc. Natl. Acad. Sci. USA, 83: 3746-3750, 1986. A temperature that is similar to (e.g., within 5° C. or within 10° C.) or equal to the proposed denaturing temperature of a probe or primer is considered to be high stringency. Medium stringency is to be considered to be within 10° C. to 20° C. or 10° C. to 15° C. of the calculated Tm of the probe or primer.

### a) Probe/Primer Design and Production

As will be apparent to the skilled artisan, the specific probe or primer used in an assay of the present invention will depend upon the assay format used. Clearly, a probe or primer that is capable of preferentially or specifically hybridizing or annealing to or detecting the marker of interest is preferred. Methods for designing probes and/or primers for, for example, PCR or hybridization are known in the art and described, for example, in Dieffenbach and Dveksler (Eds) (In: PCR Primer: A Laboratory Manual, Cold Spring Harbor Laboratories, NY, 1995). Furthermore, several software packages are publicly available that design optimal probes and/or primers for a variety of assays, e.g. Primer 3 available from the Center for Genome Research, Cambridge, Mass., USA. Probes and/or primers useful for detection of a marker associated with a therapeutic response, are assessed to determine those that do not form hairpins, self-prime or form primer dimers (e.g. with another probe or primer used in a detection assay).

Furthermore, a probe or primer (or the sequence thereof) is assessed to determine the temperature at which it denatures from a target nucleic acid (i.e. the melting temperature of the probe or primer, or Tm). Methods of determining Tm are known in the art and described, for example, in Santa Lucia, Proc. Natl. Acad. Sci. USA, 95: 1460-1465, 1995 or Bresslauer et al., Proc. Natl. Acad. Sci. USA, 83: 3746-3750, 1986.

A primer or probe useful for detecting a SNP or mutation in an allele specific PCR assay or a ligase chain reaction assay is designed such that the 3′ terminal nucleotide hybridizes to the site of the SNP or mutation. The 3′ terminal nucleotide may be any of the nucleotides known to be present at the site of the SNP or mutation. When complementary nucleotides occur in the probe or primer and at the site of the polymorphism the 3′ end of the probe or primer hybridizes completely to the marker of interest and facilitates amplification, for example, PCR amplification or ligation to another nucleic acid. Accordingly, a probe or primer that completely hybridizes to the target nucleic acid produces a positive result in an assay.

In another example, a primer useful for a primer extension reaction is designed such that it preferentially or specifically hybridizes to a region adjacent to a specific nucleotide of interest, e.g. a SNP or mutation.

Whilst the specific hybridization of a probe or primer may be estimated by determining the degree of homology of the probe or primer to any nucleic acid using software, such as, for example, BLAST, the specificity of a probe or primer can only be determined empirically using methods known in the art.

A locked nucleic acid (LNA) or protein-nucleic acid (PNA) probe or a molecular beacon useful, for example, for detection of a SNP or mutation or microsatellite by hybridization is at least about 8 to 12 nucleotides in length. Preferably, the nucleic acid, or derivative thereof, that hybridizes to the site of the SNP or mutation or microsatellite is positioned at approximately the centre of the probe, thereby facilitating selective hybridization and accurate detection.

Methods for producing/synthesizing a probe or primer of the present invention are known in the art. For example, oligonucleotide synthesis is described, in Gait (Ed) (In: Oligonucleotide Synthesis: A Practical Approach, IRL Press, Oxford, 1984). For example, a probe or primer may be obtained by biological synthesis (eg. by digestion of a nucleic acid with a restriction endonuclease) or by chemical synthesis. For short sequences (up to about 100 nucleotides) chemical synthesis is preferable.

For longer sequences standard replication methods employed in molecular biology are useful, such as, for example, the use of M13 for single stranded DNA as described by J. Messing (1983) Methods Enzymol, 101, 20-78.

Other methods for oligonucleotide synthesis include, for example, phosphotriester and phosphodiester methods (Narang, et al. Meth. Enzymol 68: 90, 1979) and synthesis on a support (Beaucage, et al Tetrahedron Letters 22: 1859-1862, 1981) as well as phosphoramidate technique, Caruthers, M. H., et al., “Methods in Enzymology,” Vol. 154, pp. 287-314 (1988), and others described in “Synthesis and Applications of DNA and RNA,” S. A. Narang, editor, Academic Press, New York, 1987, and the references contained therein.

LNA synthesis is described, for example, in Nielsen et al, J. Chem. Soc. Perkin Trans., 1: 3423, 1997; Singh and Wengel, Chem. Commun. 1247, 1998. While, PNA synthesis is described, for example, in Egholm et al., Am. Chem. Soc., 114: 1895, 1992; Egholm et al., Nature, 365: 566, 1993; and Orum et al., Nucl. Acids Res., 21: 5332, 1993.

In one example, the probe or primer comprises one or more detectable markers. For example, the probe or primer comprises a fluorescent label such as, for example, fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7, fluorescein (5-carboxyfluorescein-N-hydroxysuccinimide ester), rhodamine (5,6-tetramethyl rhodamine). The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm).

Alternatively, the probe or primer is labeled with, for example, a fluorescent semiconductor nanocrystal (as described, for example, in U.S. Pat. No. 6,306,610), a radiolabel or an enzyme (e.g. horseradish peroxidase (HRP), alkaline phosphatase (AP) or β-galactosidase).

Such detectable labels facilitate the detection of a probe or primer, for example, the hybridization of the probe or primer or an amplification product produced using the probe or primer. Methods for producing such a labeled probe or primer are known in the art. Furthermore, commercial sources for the production of a labeled probe or primer will be known to the skilled artisan, e.g., Sigma-Genosys, Sydney, Australia.

The present invention additionally contemplates the use a probe or primer as described herein in the manufacture of a diagnostic reagent for diagnosing or determining a predisposition to a therapeutic response.

### b) Detection Methods

Methods for detecting nucleic acids are known in the art and include for example, hybridization based assays, amplification based assays and restriction endonuclease based assays. For example, a change in the sequence of a region of the genome or an expression product thereof, such as, for example, an insertion, a deletion, a transversion, a transition, alternative splicing or a change in the preference of or occurrence of a splice form of a gene is detected using a method, such as, polymerase chain reaction (PCR) strand displacement amplification, ligase chain reaction, cycling probe technology or a DNA microarray chip amongst others.

Methods of PCR are known in the art and described, for example, in Dieffenbach (Ed) and Dveksler (Ed) (In: PCR Primer: A Laboratory Manual, Cold Spring Harbor Laboratories, NY, 1995). Generally, for PCR two non-complementary nucleic acid primer molecules comprising at least about 20 nucleotides in length, and more preferably at least 30 nucleotides in length are hybridized to different strands of a nucleic acid template molecule, and specific nucleic acid molecule copies of the template are amplified enzymatically. PCR products may be detected using electrophoresis and detection with a detectable marker that binds nucleic acids. Alternatively, one or more of the oligonucleotides are labeled with a detectable marker (e.g. a fluorophore) and the amplification product detected using, for example, a lightcycler (Perkin Elmer, Wellesley, Mass., USA). Clearly, the present invention also encompasses quantitative forms of PCR, such as, for example, Taqman assays.

Strand displacement amplification (SDA) utilizes oligonucleotides, a DNA polymerase and a restriction endonuclease to amplify a target sequence. The oligonucleotides are hybridized to a target nucleic acid and the polymerase used to produce a copy of this region. The duplexes of copied nucleic acid and target nucleic acid are then nicked with an endonuclease that specifically recognizes a sequence at the beginning of the copied nucleic acid. The DNA polymerase recognizes the nicked DNA and produces another copy of the target region at the same time displacing the previously generated nucleic acid. The advantage of SDA is that it occurs in an isothermal format, thereby facilitating high-throughput automated analysis.

Ligase chain reaction (described in EU 320,308 and U.S. Pat. No. 4,883,750) uses at least two oligonucleotides that bind to a target nucleic acid in such a way that they are adjacent. A ligase enzyme is then used to link the oligonucleotides. Using thermocycling the ligated oligonucleotides then become a target for further oligonucleotides. The ligated fragments are then detected, for example, using electrophoresis, or MALDI-TOF. Alternatively, or in addition, one or more of the probes is labeled with a detectable marker, thereby facilitating rapid detection.

Cycling Probe Technology uses chimeric synthetic probe that comprises DNA-RNA-DNA that is capable of hybridizing to a target sequence. Upon hybridization to a target sequence the RNA-DNA duplex formed is a target for RNase H thereby cleaving the probe. The cleaved probe is then detected using, for example, electrophoresis or MALDI-TOF.

In a preferred example, a marker that is associated with or causative of a therapeutic response, occurs within a protein coding region of a genomic gene (e.g. an IFN-Λ3 gene) and is detectable in mRNA encoded by that gene. For example, such a marker may be an alternate splice-form of a mRNA encoded by a genomic gene (e.g. a splice form not observed in a normal and/or healthy subject, or, alternatively, an increase or decrease in the level of a splice form in a subject that carries the marker). Such a marker may be detected using, for example, reverse-transcriptase PCR (RT-PCR), transcription mediated amplification (TMA) or nucleic acid sequence based amplification (NASBA), although any mRNA or cDNA based hybridization and/or amplification protocol is clearly amenable to the instant invention.

Methods of RT-PCR are known in the art and described, for example, in Dieffenbach (Ed) and Dveksler (Ed) (In: PCR Primer: A Laboratory Manual, Cold Spring Harbor Laboratories, NY, 1995).

Methods of TMA or self-sustained sequence replication (3SR) use two or more oligonucleotides that flank a target sequence, a RNA polymerase, RNase H and a reverse transcriptase. One oligonucleotide (that also comprises a RNA polymerase binding site) hybridizes to an RNA molecule that comprises the target sequence and the reverse transcriptase produces cDNA copy of this region. RNase H is used to digest the RNA in the RNA-DNA complex, and the second oligonucleotide used to produce a copy of the cDNA. The RNA polymerase is then used to produce a RNA copy of the cDNA, and the process repeated.

NASBA systems rely on the simultaneous activity of three enzymes (a reverse transcriptase, RNase H and RNA polymerase) to selectively amplify target mRNA sequences. The mRNA template is transcribed to cDNA by reverse transcription using an oligonucleotide that hybridizes to the target sequence and comprises a RNA polymerase binding site at its 5′ end. The template RNA is digested with RNase H and double stranded DNA is synthesized. The RNA polymerase then produces multiple RNA copies of the cDNA and the process is repeated.

Clearly, the hybridization to and/or amplification of a marker associated with a therapeutic response, using any of these methods is detectable using, for example, electrophoresis and/or mass spectrometry. In this regard, one or more of the probes/primers and/or one or more of the nucleotides used in an amplification reactions may be labeled with a detectable marker to facilitate rapid detection of a marker, for example, marker as described supra, e.g., a fluorescent label (e.g. Cy5 or Cy3) or a radioisotope (e.g. ³²P).

Alternatively, amplification of a nucleic acid may be continuously monitored using a melting curve analysis method, such as that described in, for example, U.S. Pat. No. 6,174,670.

In a one exemplified form of the invention, a marker associated with a therapeutic response, comprises a single nucleotide change. Methods of detecting single nucleotide changes are known in the art, and reviewed, for example, in Landegren et al, Genome Research 8: 769-776, 1998.

For example, a single nucleotide changes that introduces or alters a sequence that is a recognition sequence for a restriction endonuclease is detected by digesting DNA with the endonuclease and detecting the fragment of interest using, for example, Southern blotting (described in Ausubel et at (In: Current Protocols in Molecular Biology. Wiley Interscience, ISBN 047 150338, 1987) and Sambrook et at (In: Molecular Cloning: Molecular Cloning: A Laboratory Manual, Cold Spring Harbor Laboratories, New York, Third Edition 2001)). Alternatively, a nucleic acid amplification method described supra, is used to amplify the region surrounding the single nucleotide changes. The amplification product is then incubated with the endonuclease and any resulting fragments detected, for example, by electrophoresis, MALDI-TOF or PCR.

The direct analysis of the sequence of polymorphisms of the present invention can be accomplished using either the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

Alternatively, a single nucleotide change is detected using single stranded conformational polymorphism (SSCP) analysis. SSCP analysis relies upon the formation of secondary structures in nucleic acids and the sequence dependent nature of these secondary structures. In one form of this analysis an amplification method, such as, for example, a method described supra, is used to amplify a nucleic acid that comprises a single nucleotide change. The amplified nucleic acids are then denatured, cooled and analyzed using, for example, non-denaturing polyarcrylamide gel electrophoresis, mass spectrometry, or liquid chromatography (e.g. HPLC or dHPLC). Regions that comprise different sequences form different secondary structures, and as a consequence migrate at different rates through, for example, a gel and/or a charged field. Clearly, a detectable marker may be incorporated into a probe/primer useful in SSCP analysis to facilitate rapid marker detection.

Alternatively, any nucleotide changes are detected using, for example, mass spectrometry or capillary electrophoresis. For example, amplified products of a region of DNA comprising a single nucleotide change from a test sample are mixed with amplified products from a normal/healthy individual. The products are denatured and allowed to reanneal. Clearly those samples that comprise a different nucleotide at the position of the single nucleotide change will not completely anneal to a nucleic acid molecule from a normal/healthy individual thereby changing the charge and/or conformation of the nucleic acid, when compared to a completely annealed nucleic acid. Such incorrect base pairing is detectable using, for example, mass spectrometry.

Mass spectrometry is also useful for detecting the molecular weight of a short amplified product, wherein a nucleotide change causes a change in molecular weight of the nucleic acid molecule (such a method is described, for example, in U.S. Pat. No. 6,574,700).

Allele specific PCR (as described, for example, In Liu et al, Genome Research, 7: 389-398, 1997) is also useful for determining the presence of one or other allele of a single nucleotide change. An oligonucleotide is designed, in which the most 3′ base of the oligonucleotide hybridizes with the single nucleotide change. During a PCR reaction, if the 3′ end of the oligonucleotide does not hybridize to a target sequence, little or no PCR product is produced, indicating that a base other than that present in the oligonucleotide is present at the site of single nucleotide change in the sample. PCR products are then detected using, for example, gel or capillary electrophoresis or mass spectrometry.

Primer extension methods (described, for example, in Dieffenbach (Ed) and Dveksler (Ed) (In: PCR Primer: A Laboratory Manual, Cold Spring Harbor Laboratories, NY, 1995)) are also useful for the detection of a single nucleotide change. An oligonucleotide that hybridizes to the region of a nucleic acid adjacent to the single nucleotide change. This oligonucleotide is then used in a primer extension protocol with a polymerase and a free nucleotide diphosphate that corresponds to either or any of the possible bases that occur at the single nucleotide change. Preferably the nucleotide-diphosphate is labeled with a detectable marker (e.g. a fluorophore). Following primer extension, unbound labeled nucleotide diphosphates are removed, e.g. using size exclusion chromatography or electrophoresis, or hydrolyzed, using for example, alkaline phosphatase, and the incorporation of the labeled nucleotide into the oligonucleotide is detected, indicating the base that is present at the site of the single nucleotide change. Alternatively, or in addition, as exemplified herein primer extension products are detected using mass spectrometry (e.g. MALDI-TOF).

Clearly, the present invention extends to high-throughput forms primer extension analysis, such as, for example, minisequencing (Sy Vämen et al., Genomics 9: 341-342, 1995). In such a method, a probe or primer (or multiple probes or primers) are immobilized on a solid support (e.g. a glass slide). A biological sample comprising nucleic acid is then brought into direct contact with the probe/s or primer/s, and a primer extension protocol performed with each of the free nucleotide bases labeled with a different detectable marker. The nucleotide present at a single nucleotide change or a number of single nucleotide changes is then determined by determining the detectable marker bound to each probe and/or primer.

Fluorescently labeled locked nucleic acid (LNA) molecules or fluorescently labeled protein-nucleic acid (PNA) molecules are useful for the detection of SNPs (as described in Simeonov and Nikiforov, Nucleic Acids Research, 30 (17): 1-5, 2002). LNA and PNA molecules bind, with high affinity, to nucleic acid, in particular, DNA. Fluorophores (in particular, rhodomine or hexachlorofluorescein) conjugated to the LNA or PNA probe fluoresce at a significantly greater level upon hybridization of the probe to target nucleic acid. However, the level of increase of fluorescence is not enhanced to the same level when even a single nucleotide mismatch occurs. Accordingly, the degree of fluorescence detected in a sample is indicative of the presence of a mismatch between the LNA or PNA probe and the target nucleic acid, such as, in the presence of a SNP. Preferably, fluorescently labeled LNA or PNA technology is used to detect a single base change in a nucleic acid that has been previously amplified using, for example, an amplification method described supra.

As will be apparent to the skilled artisan, LNA or PNA detection technology is amenable to a high-throughput detection of one or more markers immobilizing an LNA or PNA probe to a solid support, as described in Orum et al., Clin. Chem. 45: 1898-1905, 1999.

Similarly, Molecular Beacons are useful for detecting single nucleotide changes directly in a sample or in an amplified product (see, for example, Mhlang and Malmberg, Methods 25: 463-471, 2001). Molecular beacons are single stranded nucleic acid molecules with a stem-and-loop structure. The loop structure is complementary to the region surrounding the single nucleotide change of interest. The stem structure is formed by annealing two “arms,” complementary to each other, that are on either side of the probe (loop). A fluorescent moiety is bound to one arm and a quenching moiety to the other arm that suppresses any detectable fluorescence when the molecular beacon is not bound to a target sequence. Upon binding of the loop region to its target nucleic acid the arms are separated and fluorescence is detectable. However, even a single base mismatch significantly alters the level of fluorescence detected in a sample. Accordingly, the presence or absence of a particular base at the site of a single nucleotide change is determined by the level of fluorescence detected.

A single nucleotide change can also be identified by hybridization to nucleic acid arrays, an example of which is described in WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

Clearly the present invention encompasses other methods of detecting a single nucleotide change that is associated with a therapeutic response, such as, for example, SNP microarrays (available from Affymetrix, or described, for example, in U.S. Pat. No. 6,468,743 or Hacia et al, Nature Genetics, 14: 441, 1996), Taqman assays (as described in Livak et al, Nature Genetics, 9: 341-342, 1995), solid phase minisequencing (as described in Syvämen et al, Genomics, 13: 1008-1017, 1992), minisequencing with FRET (as described in Chen and Kwok, Nucleic Acids Res. 25: 347-353, 1997) or pyrominisequencing (as reviewed in Landegren et al., Genome Res., 8(8): 769-776, 1998).

In a preferred example, a single nucleotide change associated with a therapeutic response, is detected using a Taqman assay essentially as described by Corder et al., Science, 261: 921-923.

### (ii) Protein Marker Detection

### a) Antibodies

Methods for detecting polypeptides generally make use of a ligand or antibody that preferentially or specifically binds to the target polypeptide. As used herein the term “ligand” shall be taken in its broadest context to include any chemical compound, polynucleotide, peptide, protein, lipid, carbohydrate, small molecule, natural product, polymer, etc. that is capable of selectively binding, whether covalently or not, to one or more specific sites on a polypeptide encoded by a gene linked to a SNP of Table 1. The ligand may bind to its target via any means including hydrophobic interactions, hydrogen bonding, electrostatic interactions, van der Waals interactions, pi stacking, covalent bonding, or magnetic interactions amongst others. It is particularly preferred that a ligand is able to specifically bind to a specific form of a polypeptide marker.

As used herein, the term “antibody” refers to intact monoclonal or polyclonal antibodies, immunoglobulin (IgA, IgD, IgG, IgM, IgE) fractions, humanized antibodies, or recombinant single chain antibodies, as well as fragments thereof, such as, for example Fab, F(ab)2, and Fv fragments.

Antibodies are prepared by any of a variety of techniques known to those of ordinary skill in the art, and described, for example in, Harlow and Lane (In: Antibodies: A Laboratory Manual, Cold Spring Harbor Laboratory, 1988). In one such technique, an immunogen comprising the antigenic polypeptide is initially injected into any one of a wide variety of animals (e.g., mice, rats, rabbits, sheep, humans, dogs, pigs, chickens and goats). The immunogen is derived from a natural source, produced by recombinant expression means, or artificially generated, such as by chemical synthesis (e.g., BOC chemistry or FMOC chemistry). A peptide comprising any variant amino acid listed in Table 1 may be employed as an antigen for antibody production.

A peptide, polypeptide or protein is joined to a carrier protein, such as bovine serum albumin or keyhole limpet hemocyanin. The immunogen and optionally a carrier for the protein is injected into the animal host, preferably according to a predetermined schedule incorporating one or more booster immunizations, and blood collected from said the animals periodically. Optionally, the immunogen is injected in the presence of an adjuvant, such as, for example Freund's complete or incomplete adjuvant, lysolecithin and dinitrophenol to enhance the subject's immune response to the immunogen. Monoclonal or polyclonal antibodies specific for the polypeptide are then purified from blood isolated from an animal by, for example, affinity chromatography using the polypeptide coupled to a suitable solid support.

Monoclonal antibodies specific for the antigenic polypeptide of interest are prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines are produced, for example, from spleen cells obtained from an animal immunized as described supra. The spleen cells are immortalized by, for example, fusion with a myeloma cell fusion partner, preferably one that is syngenic with the immunized animal. A variety of fusion techniques are known in the art, for example, the spleen cells and myeloma cells are combined with a nonionic detergent or electrofused and then grown in a selective medium that supports the growth of hybrid cells, but not myeloma cells. A preferred selection technique uses HAT (hypoxanthine, aminopterin, and thymine) selection. After a sufficient time, usually about 1 to 2 weeks, colonies of hybrids are observed. Single colonies are selected and growth media in which the cells have been grown is tested for the presence of an antibody having binding activity against the polypeptide (immunogen). Hybridomas having high reactivity and specificity are preferred.

Monoclonal antibodies are isolated from the supernatants of growing hybridoma colonies using methods such as, for example, affinity purification as described supra.

Various techniques are also known for enhancing antibody yield, such as injection of the hybridoma cell line into the peritoneal cavity of a suitable vertebrate host, such as a mouse. Monoclonal antibodies are then harvested from the ascites fluid or the blood of such an animal subject. Contaminants are removed from the antibodies by conventional techniques, such as chromatography, gel filtration, precipitation, and/or extraction. The marker associated with neurodegeneration of this invention may be used in the purification process in, for example, an affinity chromatography step.

It is preferable that an immunogen used in the production of an antibody is one which is sufficiently antigenic to stimulate the production of antibodies that will bind to the immunogen and is preferably, a high titer antibody. In one example, an immunogen is an entire protein. In another example, an immunogen consists of a peptide representing a fragment of a polypeptide. Preferably an antibody raised to such an immunogen also recognizes the full-length protein from which the immunogen was derived, such as, for example, in its native state or having native conformation.

Alternatively, or in addition, an antibody raised against a peptide immunogen recognizes the full-length protein from which the immunogen was derived when the protein is denatured. By “denatured” is meant that conformational epitopes of the protein are disrupted under conditions that retain linear B cell epitopes of the protein. As will be known to a skilled artisan linear epitopes and conformational epitopes may overlap.

Alternatively, a monoclonal antibody capable of binding to a form of an IFN-λ3 polypeptide or a fragment thereof is produced using a method such as, for example, a human B-cell hybridoma technique (Kozbar et al., Immunol. Today 4:72, 1983), a EBV-hybridoma technique to produce human monoclonal antibodies (Cole et al. Monoclonal Antibodies in Cancer Therapy, 1985 Allen R. Bliss, Inc., pages 77-96), or screening of combinatorial antibody libraries (Huse et al., Science 246:1275, 1989).

Such an antibody is then particularly useful in detecting the presence of a marker of a therapeutic response.

The methods described supra are also suitable for production of an antibody or antibody binding fragment as described herein according to any example.

### b) Detection Methods

In one example, the method of the invention detects the presence of a marker in a polypeptide, aid marker being associated or causative of with a therapeutic response.

An amount, level or presence of a polypeptide is determined using any of a variety of techniques known to the skilled artisan such as, for example, a technique selected from the group consisting of, immunohistochemistry, immunofluorescence, an immunoblot, a Western blot, a dot blot, an enzyme linked immunosorbent assay (ELISA), radioimmunoassay (RIA), enzyme immunoassay, fluorescence resonance energy transfer (FRET), matrix-assisted laser desorption/ionization time of flight (MALDI-TOF), electrospray ionization (ESI), mass spectrometry (including tandem mass spectrometry, e.g. LC MS/MS), biosensor technology, evanescent fiber-optics technology or protein chip technology.

In one example, an assay used to determine the amount or level of a protein is a semi-quantitative assay. In another example, an assay used to determine the amount or level of a protein in a quantitative assay.

Preferably, an amount of antibody or ligand bound to a marker of a therapeutic response, in an IFN-λ3 polypeptide is determined using an immunoassay. Preferably, using an assay selected from the group consisting of, immunohistochemistry, immunofluorescence, enzyme linked immunosorbent assay (ELISA), fluorescence linked immunosorbent assay (FLISA) Western blotting, RIA, a biosensor assay, a protein chip assay, a mass spectrometry assay, a fluorescence resonance energy transfer assay and an immunostaining assay (e.g. immunofluorescence).

Standard solid-phase ELISA or FLISA formats are particularly useful in determining the concentration of a protein from a variety of samples.

In one form such an assay involves immobilizing a biological sample onto a solid matrix, such as, for example a polystyrene or polycarbonate microwell or dipstick, a membrane, or a glass support (e.g. a glass slide). An antibody that specifically binds to a marker of a therapeutic response, e.g., an IFN-λ3 polypeptide or other polypeptide encoded by a gene linked to a SNP of Table 1, is brought into direct contact with the immobilized biological sample, and forms a direct bond with any of its target protein present in said sample. This antibody is generally labeled with a detectable reporter molecule, such as for example, a fluorescent label (e.g. FITC or Texas Red) or a fluorescent semiconductor nanocrystal (as described in U.S. Pat. No. 6,306,610) in the case of a FLISA or an enzyme (e.g. horseradish peroxidase (HRP), alkaline phosphatase (AP) or β-galactosidase) in the case of an ELISA, or alternatively a suitably labeled secondary antibody is used that binds to the first antibody. Following washing to remove any unbound antibody, the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal) in the case of an enzymatic label.

Such ELISA or FLISA based systems are suitable for quantification of the amount of a protein in a sample, by calibrating the detection system against known amounts of a protein standard to which the antibody binds, such as for example, an isolated and/or recombinant IFN-Λ3 polypeptide or immunogenic fragment thereof or epitope thereof.

In another form, an ELISA comprises immobilizing an antibody or ligand that specifically binds an IFN-λ3 polypeptide or other polypeptide encoded by a gene linked to a SNP of Table 1 on a solid matrix, such as, for example, a membrane, a polystyrene or polycarbonate microwell, a polystyrene or polycarbonate dipstick or a glass support. A sample is then brought into physical relation with said antibody, and a marker within the polypeptide is bound or ‘captured’. The bound protein is then detected using a labeled antibody. For example, if the marker is captured from a human sample, a labeled anti-human antibody that binds to an epitope that is distinct from the first (capture) antibody is used to detect the captured protein. Alternatively, a third labeled antibody can be used that binds the second (detecting) antibody.

It will be apparent to the skilled person that the assay formats described herein are amenable to high throughput formats, such as, for example automation of screening processes or a microarray format as described in Mendoza et al., Biotechniques 27(4): 778-788, 1999. Furthermore, variations of the above-described assay will be apparent to those skilled in the art, such as, for example, a competitive ELISA.

Alternatively, a marker within an IFN-λ3 polypeptide or other polypeptide encoded by a gene linked to a SNP of Table 1 is detected using a radioimmunoassay (RIA). The basic principle of the assay is the use of a radiolabeled antibody or antigen to detect antibody-antigen interactions. An antibody or ligand that specifically binds to the marker is bound to a solid support and a sample brought into direct contact with said antibody. To detect the level of bound antigen, an isolated and/or recombinant form of the antigen is radiolabeled and brought into contact with the same antibody. Following washing, the level of bound radioactivity is detected. As any antigen in the biological sample inhibits binding of the radiolabeled antigen the level of radioactivity detected is inversely proportional to the level of antigen in the sample. Such an assay may be quantitated by using a standard curve using increasing known concentrations of the isolated antigen.

As will be apparent to the skilled artisan, such an assay may be modified to use any reporter molecule, such as, for example, an enzyme or a fluorescent molecule, in place of a radioactive label.

In another example, Western blotting is used to determine the level of a marker within an IFN-λ3 polypeptide or other polypeptide encoded by a gene linked to a SNP of Table 1. In such an assay, protein from a sample is separated using sodium doedecyl sulphate polyacrylamide gel electrophoresis (SDS-PAGE) using techniques known in the art and described in, for example, Scopes (In: Protein Purification: Principles and Practice, Third Edition, Springer Verlag, 1994). Separated proteins are then transferred to a solid support, such as, for example, a membrane (e.g., a PVDF membrane), using methods known in the art, for example, electrotransfer. This membrane is then blocked and probed with a labeled antibody or ligand that specifically binds to a marker of a therapeutic response. Alternatively, a labeled secondary, or even tertiary, antibody or ligand is used to detect the binding of a specific primary antibody. The level of label is then determined using an assay appropriate for the label used. An appropriate assay will be apparent to the skilled artisan.

For example, the level or presence a protein marker is determined using methods known in the art, such as, for example, densitometry. In one example, the intensity of a protein band or spot is normalized against the total amount of protein loaded on a SDS-PAGE gel using methods known in the art. Alternatively, the level of the marker detected is normalized against the level of a control/reference protein. Such control proteins are known in the art, and include, for example, actin, glyceraldehyde 3-phosphate dehydrogenase (GAPDH), β2 microglobulin, hydroxy-methylbilane synthase, hypoxanthine phosphoribosyl-transferase 1 (HPRT), ribosomal protein L13c, succinate dehydrogenase complex subunit A and TATA box binding protein (TBP).

In an alternative example, a polypeptide marker of a therapeutic response is detected within a cell, using methods known in the art, such as, for example, immunohistochemistry or immunofluorescence. For example, a cell or tissue section that is to be analyzed to determine the presence of the marker is fixed, to stabilize and protect both the cell and the proteins contained within the cell. Preferably, the method of fixation does not disrupt or destroy the antigenicity of the marker, thus rendering it undetectable. Methods of fixing a cell are known in the art and include for example, treatment with paraformaldehyde, treatment with alcohol, treatment with acetone, treatment with methanol, treatment with Bouin's fixative and treatment with glutaraldehyde. Following fixation a cell is incubated with a ligand or antibody capable of binding the marker. The ligand or antibody is, for example, labeled with a detectable marker, such as, for example, a fluorescent label (e.g. FITC or Texas Red), a fluorescent semiconductor nanocrystal (as described in U.S. Pat. No. 6,306,610) or an enzyme (e.g. horseradish peroxidase (HRP)), alkaline phosphatase (AP) or β-galactosidase. Alternatively, a second labeled antibody that binds to the first antibody is used to detect the first antibody. Following washing to remove any unbound antibody, the level of the bound to said labeled antibody is detected using the relevant detection means. Means for detecting a fluorescent label will vary depending upon the type of label used and will be apparent to the skilled artisan.

Optionally, immunofluorescence or immunohistochemistry will comprise additional steps such as, for example, cell permeabilization (using, for example, n-octyl-βD-glucopyranoside, deoxycholate, a non-ionic detergent such as Triton X-100 NP-40, low concentrations of ionic detergents, such as, for example SDS or saponin) and/or antigen retrieval (using, for example, heat).

Methods using immunofluorescence are preferable, as they are quantitative or at least semi-quantitative. Methods of quantitating the degree of fluorescence of a stained cell are known in the art and described, for example, in Immunohistochemistry (Cuello, 1984 John Wiley and Sons, ASIN 0471900524).

Biosensor devices generally employ an electrode surface in combination with current or impedance measuring elements to be integrated into a device in combination with the assay substrate (such as that described in U.S. Pat. No. 5,567,301). An antibody/ligand that specifically binds to a marker of a therapeutic response is preferably incorporated onto the surface of a biosensor device and a biological sample contacted to said device. A change in the detected current or impedance by the biosensor device indicates protein binding to said antibody. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several proteins or peptides in a small amount of body fluids.

Evanescent biosensors are also preferred as they do not require the pretreatment of a biological sample prior to detection of a protein of interest. An evanescent biosensor generally relies upon light of a predetermined wavelength interacting with a fluorescent molecule, such as for example, a fluorescent antibody attached near the probe's surface, to emit fluorescence at a different wavelength upon binding of the target polypeptide to the antibody or ligand.

Micro- or nano-cantilever biosensors are also preferred as they do not require the use of a detectable label. A cantilever biosensor utilizes a ligand and/or antibody capable of specifically detecting the analyte of interest that is bound to the surface of a deflectable arm of a micro- or nano-cantilever. Upon binding of the analyte of interest (e.g. a marker within an IFN-λ3 polypeptide or other polypeptide encoded by a gene linked to a SNP of Table 1) the deflectable arm of the cantilever is deflected in a vertical direction (i.e. upwards or downwards). The change in the deflection of the deflectable arm is then detected by any of a variety of methods, such as, for example, atomic force microscopy, a change in oscillation of the deflectable arm or a change in pizoresistivity. Exemplary micro-cantilever sensors are described in USSN 20030010097.

To produce protein chips, the proteins, peptides, polypeptides, antibodies or ligands that are able to bind specific antibodies or proteins of interest are bound to a solid support such as for example glass, polycarbonate, polytetrafluoroethylene, polystyrene, silicon oxide, metal or silicon nitride. This immobilization is either direct (e.g. by covalent linkage, such as, for example, Schiff's base formation, disulfide linkage, or amide or urea bond formation) or indirect. Methods of generating a protein chip are known in the art and are described in for example U.S. Patent Application No. 20020136821, 20020192654, 20020102617 and U.S. Pat. No. 6,391,625. To bind a protein to a solid support it is often necessary to treat the solid support so as to create chemically reactive groups on the surface, such as, for example, with an aldehyde-containing silane reagent. Alternatively, an antibody or ligand may be captured on a microfabricated polyacrylamide gel pad and accelerated into the gel using microelectrophoresis as described in, Arenkov et al. Anal. Biochem. 278:123-131, 2000.

A protein chip may comprise only one protein, ligand or antibody, and be used to screen one or more patient samples for the presence of one polypeptide of interest. Such a chip may also be used to simultaneously screen an array of patient samples for a polypeptide of interest.

Preferably, a protein sample to be analyzed using a protein chip is attached to a reporter molecule, such as, for example, a fluorescent molecule, a radioactive molecule, an enzyme, or an antibody that is detectable using methods known in the art. Accordingly, by contacting a protein chip with a labeled sample and subsequent washing to remove any unbound proteins the presence of a bound protein is detected using methods known in the art, such as, for example, using a DNA microarray reader.

Alternatively, biomolecular interaction analysis-mass spectrometry (BIA-MS) is used to rapidly detect and characterize a protein present in complex biological samples at the low- to sub-fmole level (Nelson et al. Electrophoresis 21: 1155-1163, 2000). One technique useful in the analysis of a protein chip is surface enhanced laser desorption/ionization-time of flight-mass spectrometry (SELDI-TOF-MS) technology to characterize a protein bound to the protein chip. Alternatively, the protein chip is analyzed using ESI as described in U.S. Patent Application 20020139751.

As will be apparent from the preceding discussion, it is particularly preferred to employ a detection system that is antibody or ligand based as such assays are amenable to the detection of a marker of a therapeutic response, within an IFN-Λ3 polypeptide. Immunoassay formats are even more particularly preferred.

### Biological Samples

As examples of the present invention are based upon detection of a marker in genomic DNA any cell or sample that comprises genomic DNA is useful for determining a disease or disorder and/or a predisposition to a disease or disorder. Preferably, the cell or sample is derived from a human. Preferably, comprises a nucleated cell.

Preferred biological samples include, for example, whole blood, serum, plasma, peripheral blood mononuclear cells (PBMC), a buffy coat fraction, saliva, urine, a buccal cell, urine, fecal material, sweat, liver biopsy or a skin cell.

In a preferred example, a biological sample comprises a white blood cell, more preferably, a lymphocyte cell.

Alternatively, the biological sample is a cell isolated using a method selected from the group consisting of amniocentesis, chorionic villus sampling, fetal blood sampling (e.g. cordocentesis or percutaneous umbilical blood sampling) and other fetal tissue sampling (e.g. fetal skin biopsy). Such biological samples are useful for determining the predisposition of a developing embryo to a therapeutic response.

As will be apparent to the skilled artisan, the size of a biological sample will depend upon the detection means used. For example, an assay, such as, for example, PCR or single nucleotide primer extension may be performed on a sample comprising a single cell, although greater numbers of cells are preferred. Alternative forms of nucleic acid detection may require significantly more cells than a single cell. Furthermore, protein-based assays require sufficient cells to provide sufficient protein for an antigen based assay.

Preferably, the biological sample has been derived or isolated or obtained previously from the subject. Accordingly, the present invention also provides an ex vivo method. In one example, the method of the invention additionally comprises isolating, obtaining or providing the biological sample.

In one example, the method is performed using an extract from a biological sample, such as, for example, genomic DNA, mRNA, cDNA or protein.

As the present invention also includes detection of a marker in a IFN-λ3 gene that is associated with a disease or disorder in a cell (e.g. using immunofluorescence), the term “biological sample” also includes samples that comprise a cell or a plurality of cells, whether processed for analysis or not.

As will be apparent from the preceding description, such an assay may require the use of a suitable control, e.g. a normal individual or a typical population, e.g., for quantification.

As used herein, the term “normal individual” shall be taken to mean that the subject is selected on the basis that they are not undergoing treatment with an immunomodulatory composition.

For example, the normal subject has not been diagnosed with any form of medical condition for which therapy would be recommended using, for example, clinical analysis. Alternatively, or in addition, a suitable control sample is a control data set comprising measurements of the marker being assayed for a typical population of subjects known not to suffer from a medical condition for which therapy would be recommended. Preferably the subject is not at risk of developing such a medical condition and e.g., the subject does not have a history of the disease.

In the present context, the term “typical population” with respect to subjects known not to suffer from a disease or disorder and/or comprise or express a marker of a therapeutic response, shall be taken to refer to a population or sample of subjects tested using, for example, known methods for diagnosing the therapeutic response, and determined not to suffer from the disease and/or tested to determine the presence or absence of a marker of the disease, wherein said subjects are representative of the spectrum of normal and/or healthy subjects or subjects known not to suffer from the disease.

In one example, a reference sample is not included in an assay. Instead, a suitable reference sample is derived from an established data set previously generated from a typical population. Data derived from processing, analyzing and/or assaying a test sample is then compared to data obtained for the sample population.

Data obtained from a sufficiently large number of reference samples so as to be representative of a population allows the generation of a data set for determining the average level of a particular parameter. Accordingly, the amount of an expression product that is diagnostic of a therapeutic response can be determined for any population of individuals, and for any sample derived from said individual, for subsequent comparison to levels of the expression product determined for a sample being assayed. Where such normalized data sets are relied upon, internal controls are preferably included in each assay conducted to control for variation.

Methods for Determining a Marker Associated with Therapeutic Response

In another example, the invention additionally comprises determining a marker for a therapeutic response to any form of medical condition for which therapy with an immunomodulator would be recommended.

Given the tight association of the human IFN-λ3 gene or other gene listed in table 1 to a therapeutic response, and the provision of several markers associated with a therapeutic response, the present invention further provides methods for identifying new markers for a therapeutic response.

Accordingly, the present invention additionally provides a method for identifying a marker that is associated with a therapeutic response, said method comprising:

(i) identifying a polymorphism or allele or mutation within an IFN-λ3 gene or other gene listed in table 1 or an expression product thereof;
(ii) analyzing a panel of subjects to determine those that suffer from a condition treatable by an immunomodulatory composition and to which the immunomodulatory composition is administered, wherein not all members of the panel comprise the polymorphism or allele or mutation; and
(iii) determining the variation in the development of the therapeutic response to the immunomodulatory composition, wherein said variation indicates that the polymorphism or allele or mutation is associated with a subject's response.

Methods for determining associations are known in the art and reviewed, for example, in King (Ed) Rotter (Ed) and Motulski (Ed), The Genetic Basis of Common Disease, Oxford University Press, 2nd Edition, ISBN 0195125827, and Miller and Cronin (Eds), Genetic Polymorphisms and Susceptibility to Disease, Taylor and Francis, 1st Edition, ISBN 0748408223.

Generally, determining an association between a marker (e.g. a polymorphism and/or allele and/or a splice form and/or a mutation) and an event e.g., a response, involves comparing the frequency of a polymorphism, allele, splice form or mutation at a specific locus between a sample of unrelated individuals undergoing treatment (i.e., and an appropriate control that is representative of the allelic distribution in the normal population.

Several methods are useful for determining associations, however such studies should consider several parameters to avoid difficulties, such as, for example, population stratification, that may produce false positive results.

Population stratification occurs when there are multiple subgroups with different allele frequencies present within a population. The different underlying allele frequencies in the sampled subgroups may be independent of the disease, disorder and/or phenotype within each group, and, as a consequence, may produce erroneous conclusions of linkage disequilibrium or association.

Generally, problems of population stratification are avoided by using appropriate control samples. For example, case-comparison based design may be used in which a comparison between a group of unrelated probands with the disease, disorder and/or phenotype and a group of control (comparison) individuals who are unrelated to each other or to the probands, but who have been matched to the proband group on relevant variable (other than affection status) that may influence genotype (e.g. sex, ethnicity and/or age).

Alternatively, controls are screened to exclude those subjects that have a personal history of a disease or treatment. Such a “supernormal” control group is representative of the allele distribution of individuals unaffected by a disease or treatment.

In general, an analysis of association is used to detect non-random distribution of one or more alleles and/or polymorphisms and/or splice variants within subjects affected by a disease/disorder and/or phenotype of interest. The comparison between the test population and a suitable control population is made under the null hypothesis assumption that the locus to which the alleles and/or polymorphisms are linked has no influence on phenotype, and from this a nominal p-value is produced. For analysis of a biallelic polymorphism or mutation (e.g. a SNP) using a case control study, a chi-square analysis (or equivalent test) of a 2×2 contingency table (for analysis of alleles) or a 3×2 contingency table (for analysis of genotypes) is used.

For analysis using a family-based association study, marker data from members of the family of each proband are used to estimate the expected null distributions and an appropriate statistical test performed that compares observed data with that expected under the null hypothesis.

Another method useful in the analysis of association of a marker with a disease, disorder and/or phenotype is the genomic control method (Devlin and Roeder, Biometrics, 55: 997-1004, 1999). For a case-control analysis of candidate allele/polymorphism the genetic control method computes chi-square test statistics for both null and candidate loci. The variability and/or magnitude of the test statistics observed for the null loci are increased if population stratification and/or unmeasured genetic relationships among the subjects exist. This data is then used to derive a multiplier that is used to adjust the critical value for significance test for candidate loci. In this manner, genetic control permits analysis of stratified case-control data without an increased rate of false positives.

A structured association approach (Pritchard et al., Am. J. Hum. Genet., 67: 170-181, 2000) uses marker loci unlinked to a candidate marker to infer subpopulation membership. Latent class analysis is used to control for the effect of population substructure. Essentially, null loci are used to estimate the number of subpopulations and the probability of a subject's membership to each subpopulation. This method is then capable of accounting for a change in allele/polymorphism frequency as a result of population substructure.

Alternatively, or in addition, a Bayesian statistical approach may be used to determine the significance of an association between an allele and/or polymorphism in a gene and a response to treatment. Such an approach takes account of the prior probability that the locus under examination is involved in the therapeutic response of interest (e.g., Morris et al., Am. J. Hum. Genet., 67: 155-169, 2001).

Publicly available software may be employed to determine associations.

### Formulations

An IFN compound of the invention as described herein according to any embodiment is formulated for therapy or prophylaxis with a carrier or excipient e.g., suitable for inhalation or injection. Such formulations may be administered with another immunomodulatory composition e.g., sequentially or simultaneously. Such co-administration may be in the same formulation if both active agents are amendable to formulation and administration by the same route. For example, IFNs are generally formulated as injectables, whereas guanosine analogs may be inhalable, injectable or oral formulations. Accordingly, injectable formulations e.g., for administration by subcutaneous, intramuscular, intravenous or intradermal route, are particularly preferred. Such formulations can be prepared by any method known in the art of pharmacy, for example by bringing into association the active ingredient with the carrier(s), diluent(s) or excipient(s).

Formulation of a pharmaceutical compound will vary according to the route of administration selected (e.g., solution, emulsion, capsule). For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils, for instance. Intravenous vehicles can include various additives, preservatives, or fluids, nutrient or electrolyte replenishers and the like (See, generally, Remington's Pharmaceutical Sciences, 17th Edition, Mack Publishing Co., Pa., 1985). For inhalation, the agent can be solubilized and loaded into a suitable dispenser for administration (e.g., an atomizer, nebulizer or pressurized aerosol dispenser).

To prepare such pharmaceutical formulations, one or more compounds of the present invention is/are mixed with a pharmaceutically acceptable carrier or excipient for example, by mixing with physiologically acceptable carriers, excipients, or stabilizers in the form of, e.g., lyophilized powders, slurries, aqueous solutions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

As will be apparent to a skilled artisan, a compound that is active in vivo is particularly preferred. A compound that is active in a human subject is even more preferred. Accordingly, when manufacturing a compound that is useful for the treatment of a disease it is preferable to ensure that any components added to the formulation do not inhibit or modify the activity of the active compound.

Pharmaceutical formulations may be presented in unit dose forms containing a predetermined amount of active ingredient per unit dose. Such a unit may contain for example 1 μg to 10 ug, such as 0.01 mg to 1000 mg, or 0.1 mg to 250 mg, of a compound of Structural Formula I, Structural Formula II, Structural Formula III or Structural Formula IV, depending on the condition being treated, the route of administration and the age, weight and condition of the patient.

### a) Injectable Formulations

Pharmaceutical formulations adapted for parenteral administration include aqueous and non-aqueous sterile injection solutions which may contain the antioxidants as well as buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Formulation of a modulator or compound of the present invention in an intravenous lipid emulsion or a surfactant micelle or polymeric micelle (see., e.g., Jones et al., Eur. J. Pharmaceutics Biopharmaceutics 48, 101-111, 1999; Torchilin J. Clin, release 73, 137-172, 2001; both of which are incorporated herein by reference) is particularly preferred.

Sustained release injectable formulations are produced e.g., by encapsulating the modulator or compound in porous microparticles which comprise a pharmaceutical agent and a matrix material having a volume average diameter between about 1 μm and 150 μm, e.g., between about 5 μm and 25 μm diameter. In one embodiment, the porous microparticles have an average porosity between about 5% and 90% by volume. In one embodiment, the porous microparticles further comprise one or more surfactants, such as a phospholipid. The microparticles may be dispersed in a pharmaceutically acceptable aqueous or non-aqueous vehicle for injection. Suitable matrix materials for such formulations comprise a biocompatible synthetic polymer, a lipid, a hydrophobic molecule, or a combination thereof. For example, the synthetic polymer can comprise, for example, a polymer selected from the group consisting of poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers, derivatives and blends thereof. In a preferred embodiment, the synthetic polymer comprises a poly(lactic acid), a poly(glycolic acid), a poly(lactic-co-glycolic acid), or a poly(lactide-co-glycolide).

### b) Inhalable Formulations

Pharmaceutical formulations adapted for administration by inhalation include fine particle dusts or mists which may be generated by means of various types of metered dose pressurized aerosols, nebulizers or insufflators.

Spray compositions may, for example, be formulated as aerosols delivered from pressurized packs, such as a metered dose inhaler, with the use of a suitable liquified propellant.

Capsules and cartridges for use in an inhaler or insufflator, for example gelatine, may be formulated containing a powder mix for inhalation of a compound of the invention and a suitable powder base such as lactose or starch.

Aerosol formulations are preferably arranged so that each metered dose or “puff” of aerosol contains about 0.001 μg to about 2000 μg of modulator or compound of the invention.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close to the nose. Suitable formulations wherein the carrier is a liquid, for administration as a nasal spray or as nasal drops, include aqueous or oil solutions of the active ingredient.

The overall daily dose and the metered dose delivered by capsules and cartridges in an inhaler or insufflator will generally be double those with aerosol formulations.

### Treatment Regimes

An IFN-λ2/3 compound of the invention as described according to any example hereof is formulated for therapy or prophylaxis with a carrier or excipient as described, and administered to a subject in need thereof by any means suitable e.g., by inhalation or injection. Selecting an administration regimen for a therapeutic composition depends on several factors, including the serum or tissue turnover rate of the entity, the level of symptoms, the immunogenicity of the entity, and the accessibility of the target cells in the biological matrix. Preferably, an administration regimen maximizes the amount of therapeutic compound delivered to the patient consistent with an acceptable level of side effects. Accordingly, the amount of composition delivered depends in part on the particular entity and the severity of the condition being treated.

A compound can be provided, for example, by continuous infusion, or by doses at intervals of, e.g., one day, one week, or 1-7 times per week. Doses of a composition may be provided intravenously, subcutaneously, topically, orally, nasally, rectally, intramuscularly, intracerebrally, vaginally or by inhalation. A preferred dose protocol is one involving the maximal dose or dose frequency that avoids significant undesirable side effects. A total weekly dose depends on the type and activity of the compound being used. For example, such a dose is at least about 0.05 μg/kg body weight, or at least about 0.2 μg/kg, or at least about 0.5 μg/kg, or at least about 1 μg/kg, or at least about 10 μg/kg, or at least about 100 μg/kg, or at least about 0.2 mg/kg, or at least about 1.0 mg/kg, or at least about 2.0 mg/kg, or at least about 10 mg/kg, or at least about 25 mg/kg, or at least about 50 mg/kg (see, e.g., Yang, et al. New Engl. J. Med. 349:427-434, 2003; or Herold, et al. New Engl. J. Med. 346:1692-1698, 2002.

An effective amount of a compound for a particular patient may vary depending on factors such as the condition being treated, the overall health of the patient, the method route and dose of administration and the severity of side effects, see, e.g., Maynard, et al. (1996) A Handbook of SOPs for Good Clinical Practice, Interpharm Press, Boca Raton, Fla.; or Dent (2001) Good Laboratory and Good Clinical Practice, Urch Publ., London, UK.

Determination of the appropriate dose is made by a clinician, e.g., using parameters or factors known or suspected in the art to affect treatment or predicted to affect treatment. Generally, the dose begins with an amount somewhat less than the optimum dose and is increased by small increments thereafter until the desired or optimum effect is achieved relative to any negative side effects. Important diagnostic measures include those of symptoms of the disease and/or disorder being treated. Preferably, a compound that will be used is derived from or adapted for use in the same species as the subject targeted for treatment, thereby minimizing a humoral response to the reagent.

An effective amount of therapeutic will decrease disease symptoms, for example, as described supra, typically by at least about 10%; usually by at least about 20%; preferably at least about 30%; more preferably at least about 40%, and more preferably by at least about 50%.

The route of administration is preferably by, e.g., injection or infusion by intravenous, intraperitoneal, intracerebral, intramuscular, intraocular, intraarterial, intracerebrospinal, intralesional, or pulmonary routes, or by sustained release systems or an implant (see, e.g., Sidman et al. Biopolymers 22:547-556, 1983; Langer, et al. J. Biomed. Mater. Res. 15:167-277, 1981; Langer Chem. Tech. 12:98-105, 1982; Epstein, et al. Proc. Natl. Acad. Sci. USA 82:3688-3692, 1985; Hwang, et al. Proc. Natl. Acad. Sci. USA 77:4030-4034, 1980; U.S. Pat. Nos. 6,350,466 and 6,316,024).

The present invention is further described with referenced to the following non-limiting examples.

### Example 1

### Identification of SNPs Associated with Therapy of Chronic Hepatitis C Using an Immunomodulatory Composition Comprising an Interferon (IFN)

### Summary

This example demonstrates SNPs and alleles of the present invention that are associated with a response to therapy for hepatitis C virus infection using a composition comprising IFN.

For example, the data herein demonstrate that a high response (HR) allele (approximate p value less than about 10⁻³) i.e., an allele associated with a rapid or strong or other significant response of Caucasians (Table 2) to hepatitis C therapy using IFN-α and ribavirin, as determined by virus clearance (Tables 1 and 3-6). This HR allele is an allele of the chromosome 19 SNP designated rs8099917. The corresponding low response (LR) allele i.e., associated with a poor or low response or no response to therapy, has p>0.05 in this patient cohort (Tables 1-6). The chromosome 19 SNP rs8099917 maps to position 19q13.13, in the 5′-end of the IFN-λ3 (IL28B) gene.

The inventors subsequently identified other SNPs linked to a 5 kb region of 19q13.13, between 44,420,000 and position 44,440,000 and more specifically between about position 44,423,000 and about position 44,436,000, encompassing the IFN-λ3 (IL28B) gene (Tables 1 and 3). For example, HR alleles (approximate p value less than about 10⁻³) and/or LR alleles (approximate p value greater than about 0.05) have been identified for SNPs in this region designated rs8109886, rs10853727, rs8103142 and rs12980275 (Table 4). Weak alleles were also identified for the SNPs rs4803224, rs12980602 and rs10853728 in this region (Table 4).

The IL28B association data are confirmed in subjects homozygous for one or more chromosome 19 SNPs showing strong associations with response to therapy e.g., rs8099917 in the 5′-end of the gene and/or rs8103142 in exon 2 and/or rs12980275 in the 3′-end of the gene (Tables 4 and 5). For example, double-homozyotes for the HR alleles of rs12980275 and rs8099917, and double-homozyotes for the HR alleles of rs8103142 and rs8099917 and triple homozygotes for the HR alleles of rs12980275, rs8103142 and rs8099917 show strong responses to therapy (p<6×10⁻⁴), whereas the corresponding homozygotes for LR alleles at these loci demonstrate consistently low responses to therapy (p>0.04), as shown in Table 5. In another example, haplotype data for the SNPs rs12980275, rs8105790, rs8103142, rs10853727, rs8109886 and rs8099917 also show that the presence of HR alleles at all six loci is associated consistently and significantly enhanced response to therapy (p value less than 10⁻³) whereas LR alleles at all six loci are associated consistently and significantly with poor response to therapy (p value greater than 0.25). These data support the inventors' conclusion that variations in 19q13.13 between position 44,420,000 and position 44,440,000 and more specifically between about position 44,423,000 and about position 44,436,000, such as those linked to the IL28B gene, contribute to the variation in response to therapy with an immunomodulatory composition. The instant association between variations in the IL28B gene is sufficiently-strong to indicate that genotypes in 19q13.13 between position 44,423,000 and position 44,436,000, especially IFN-λ3 (IL-28B) genotypes, can be used to predict drug responses. The data also support the use of IFN-λ e.g., IFN-λ1 and/or IFN-λ2 and/or IFN-λ3, for treatment of HCV infection and other diseases currently treated using other IFNs such as IFN-α or IFN-β or combinations thereof.

The data also demonstrate HR alleles (approximate p value less than about 10⁻⁴) and/or LR alleles (p value>0.01) for one or more SNPs at the following chromosomal locations:
a) Chromosome 1, at about 1p35 e.g., between WASF2 and ADHC1 genes; and/or
b) Chromosome 3, between about 3p21.2 and about 3p21.31 e.g., within the CACNA2D3 gene such as within an intron of the CACNA2D3 gene, and/or between about 3p24.3 and about 3p25.1 e.g., within the RTFN-1 gene such as within an intron of the RTFN-1 gene; and/or
c) Chromosome 4 at about 4q32, and/or at about 4p13, and/or at about 4p16.1 e.g., near to the CTBP1 gene; and/or
d) Chromosome 6, between about 6p12.2 and about 6p12.3 e.g., within the PHKD-1 gene such as within an intron of PHKD-1, and/or between about 6p21.33 and about 6p22 e.g., within an HLA gene cluster such as between HLA pseudogenes HCP5P10 and MICF, and/or between about 6p22.1 and about 6p22.2 e.g., between ALDH5A1 and PRL genes, and/or at about 6q13 e.g., within the RIMS-1 gene such as within an intron of the RIMS-1 gene, and/or at about 6q22.31 e.g., between C6orf68 and SLC35F1; and/or
e) Chromosome 8 between about 8q12.2 to about 8q13.1 e.g., between CRH and MGC33510 such as between ADHFE1 and MGC33510 or between RRS1 and CRH; and/or
f) Chromosome 9, between about 9q22.1 and about 9q22.2 e.g., in an intergenic region; and/or
g) Chromosome 10, between about 10q26.2 and about 10q26.3 e.g., between NPS and DOCK1; and/or
h) Chromosome 11, at about 11q21 e.g., between KIAA1731 and FN5 genes, and/or at about 11q22.3 e.g., within the CASP-1 gene such as within an intron of the CASP-1 gene; and/or
i) Chromosome 14, between about 14q22.1 and 14q22.2 e.g., between DACT1 and LOC729646; and/or
j) Chromosome 16, between about 16q23.1 and about 16q23.2 e.g., in the WWOX gene such as within an intron of the WWOX gene, and/or between about 16p11.2 and about 16p12.1 e.g., between IL21R and GFT3C1; and/OR
k) Chromosome 20, between about 20q13.12 and about 20q13.13 e.g., in the SULF2 gene such as in an intron of the SULF2 gene.

These additional SNPs and their associations are shown in Tables 1-4.

These data provide the means for predicting outcome of therapy to immunomodulatory compositions with accuracy in more than 90% of cases.

### Patient Cohorts

For stage one genotyping, a well-characterised Australian population of 302 patients of northern European ancestry, matched for age, BMI, viral titre, and treatment regime was employed (Table 2). Patients were excluded from the study if they had been co-infected with either HBV or HIV or if they were not of northern European descent. All patients included in this study had been diagnosed as infected with genotype 1 HCV based on serology and viral DNA tests, had received a standard course of pegylated interferon-alpha (IFN-α) and ribavirin, and their six-month post-treatment responses to therapy as determined by virus clearance had been determined. All patients who responded to therapy, and most patients classified as having a non-sustained viral response (“non-SVR”), had received treatment for 12 months. A few non-SVR cases received only 4 months of therapy, because they showed no reduction in viral RNA at week 12. All patients were seen by experienced hepatologists at their respective hospitals.

A larger independent cohort consisting of about 600 northern Europeans from the United Kingdom, Germany, Italy and Australia was also employed for stage two genotyping (Table 2). The criteria for recruitment of study subjects in this cohort were the same for the Australian cohort.

### Sample Collection and Processing

Australian samples for both stages were collected at Sydney (Westmead Hospital, Nepean Hospital, St Vincent's Hospital and Prince of Wales Hospital) and Brisbane (Princess Alexandra Hospital). Case samples for the replication cohorts were collected at Universtat Zu Berlin, Germany (n=298), Rheinische Friedrich-Wilhelms-Universitat, Bonn, Germany (n=43), Universita degli Studi di Turino, Turin, Italy (n=93) and Freeman Hospital, Newcastle, UK (n=91).

Blood was collected into EDTA tubes (Australian cohort). Extracted DNA normalised to 50 ng/ul was obtained for other cohorts. Genomic DNA was extracted by standard protocols. DNA quality was assessed by calculating absorbance ratio OD_{260 nm/280 nm} using nanodrop.

### Ethics Approval

Ethical approval for this study was given by Sydney West Area Health Service Human Research Ethics Committee at Westmead Hospital and the University of Sydney (HREC No. 2002/12/4.9 (1564)). All other sites had ethical approval from their respective ethics committees. Written informed consent was obtained from all participants.

### Statistical Analysis

Hardy-Weinberg equilibrium and allelic distributions in subjects having high response(s) or low response(s) were compared using a chi-squared test in Haploview version 3.31 of the Broad Institute, USA e.g., as described by Barrett et al. Bioinformatics 21, 263-265 (2005). The threshold for genome-wide significant association was set at p<1.6×10⁻⁷ i.e., 0.05/312,000. SNPs having 1.6×10⁻⁷≦p<1.0×10⁻⁴ were considered to show a highly suggestive association with response to therapy. SNPs having 1.0×10⁻⁴≦p<1.0×10⁻³ were considered to show a moderately suggestive association with response to therapy. The Cochran-Armitage trend test was used to assess association of all SNPs tested in Stage one and Stage two, and merged p values were determined.

### SNP Genotyping

A two-stage approach essentially as described by Saito et al. J. Hum. Genetics 47, 360-365 (2002) was employed for SNP genotyping using HumanLinkage panels for Infinium and GoldenGate SNP Genotyping (Illumina, Inc., San Diego, USA). SNPs on Chr 19 were fine-mapped using the Sequenom mass array iPlex genotyping platform (Sequenome, Inc, San Diego, USA). The two-stage approach was favoured as it was calculated to have a power of 87% to detect risk factors of 1.5 for disease allele frequency of 0.2 (Skol et al, Nature Genetics 38, 209-213 (2006).

### a) Stage One Genotyping

The 302 patient samples were genotyped using the Infinium HumanHap300 or CNV370 genotyping BeadChip (Illumina, Inc., San Diego, USA). Samples having a very low call rate using the Illumina cluster (i.e., genotyping efficiency less than 95%) was deleted. A minor allele frequency (MAF) check was performed for data handling accuracy, and those SNPs occurring in less than 0.05% of samples were deleted. Samples providing a Hardy-Weinberg equilibrium p value>10⁻⁴ were retained. Two individuals were excluded due to genotyping call rates less than 90%, and IBS/IBD analysis revealed that those two individuals were related. The 99% confidence interval (CI) for genotyping error was estimated to be between 1.7% and 1.8%. As ethnicity was determined by self-identification or parental ethnic identification, an assessment for possible population stratification was performed using EIGENSOFT software, essentially as described by Price et al. Nature Genetics 38, 904-909 (2006), applying principal component analysis to the genotype data to infer the axes of variation. This resulted in exclusion of five individuals from further analysis. Accordingly, the final genome-wide association (GWA) study consisted of 293 patients (162 having low response(s) (LR) and 131 having high response(s) (HR) as indicated in Table 2.

A Manhattan plot of signal intensity relative to genome position and a Quantile-Quantile plot of allelic associations for the stage one SNPs (not shown), identified SNPs that were more associated than would be expected by mere chance. A genomic inflation factor lambda of 1.005 in that analysis indicated a low possibility of false positive associations e.g., due to population stratification. A total of 312,000 SNPs passed the first stage quality filters and were analysed further. A total of 695 SNPs (0.22%) did not pass the first stage quality filters and were excluded from subsequent analysis.

From these 312,000 SNPs, SNPs were classified as highly or suggestively associated with the therapeutic response, as described under “statistical analysis” supra.

In stage one, three chromosome 19 SNPs were identified having a high or suggestive association with therapeutic response that were linked to the interferon lambda-3 (IFN-λ3) gene. No other SNPs mapping to chromosome 19 satisfied the threshold for genome-wide associations. The genomic sequences flanking these three SNPs, their chromosomal positions and locations within the IFN-λ3 gene are presented in Table 3. Two SNPs flanking the IFN-λ3 gene i.e., rs8099917 mapping to the 5′-end of IFN-λ3 (p=7.06×10⁻⁰⁸), and rs12980275 mapping to the 3′-end of IFN-λ3 (p=4.81×10⁻⁸) were well-below the threshold for significant associations with therapeutic response in stage one (Table 4). The third SNP i.e., rs8109886 mapping to the 5′-end of IFN-λ3 (p=1.29×10⁻⁰⁴) was considered to have a suggestive association with therapeutic response in stage one (Table 4).

SNPs on other chromosomes were also identified having at least moderately suggestive positive associations with therapeutic response that mapped to the following chromosomal locations:
a) rs7512595 at about 1p35, between WASF2 and ADHC1 genes;
b) rs6806020 between about 3p21.2 and about 3p21.31, within an intron of the CACNA2D3 gene; and rs12486361 between about 3p24.3 and about 3p25.1, within an intron of the RTFN-1 gene;
c) rs10018218 at about 4q32; rs1581096 at about 4p13; and rs1250105 at about 4p16.1 near to the CTBP1 gene;
d) rs7750468 at about 6q22.31, between C6orf68 and SLC35F1; rs2746200 at about 6q13, within an intron of the RIMS-1 gene; rs927188 between about 6p12.2 and about 6p12.3, within an intron of PHKD-1; rs2517861 between about 6p21.33 and about 6p22 and between HLA pseudogenes HCP5P10 and MICF; and rs2025503 and rs2066911 between about 6p22.1 and about 6p22.2, and between ALDH5A1 and PRL genes;
e) rs10283103 and rs2114487 between about 8q12.2 to about 8q13.1 e.g., between CRH and MGC33510 such as between ADHFE1 and MGC33510 or between RRS1 and CRH;
f) rs1002960 between about 9q22.1 and about 9q22.2, in an intergenic region;
g) rs1931704 between about 10q26.2 and about 10q26.3 and between NPS and DOCK1;
h) rs1939565 at about 11q21, between KIAA1731 and FN5 genes; rs568910 and rs557905 within introns of the CASP-1 gene, wherein rs568910 is in intron 2 and rs557905 is in intron 6;
i) rs1931704 between about 14q22.1 and 14q22.2, between DACT1 and LOC729646;
j) rs3093390 between about 16p11.2 and about 16p12.1 and between IL21R and GFT3C1; and rs7196702 between about 16q23.1 and about 16q23.2, in an intron of the WWOX gene; and
k) rs4402825, between about 20q13.12 and about 20q13.13 in an intron of the SULF2 gene.

These additional SNPs and their associations are shown in Tables 1-4. Of these SNPs, rs7750468, rs2066911, rsrs6806020, rs2114487 and rs1931704 were considered highly suggestive of an association, and the remaining considered to be moderately suggestive of an association based on stage one screening data. The SNP designated rs1931704 has a very close association with therapeutic response (p=4.42×10⁻⁰⁷), and was shown to be closely-linked to the neuropeptide S(NPS) gene.

A total of 512 highly and moderately associated SNPs were selected from stage one.

### b) Stage Two Genotyping

In the second stage whole genome screen, 307 SNPs having a significance level of p<1.0×10⁻⁴ irrespective of their genome location, and 206 SNPs linked to genes classified as immune regulatory or anti-viral by gene ontology and having a significance level of 1.0×10⁻⁴≦p<1.0×10⁻³ were included. The SNPs were genotyped using Golden Gate technology (Illumina, Inc., San Diego, USA). Two (2) cases having call rates of less than 0.90, 8 samples with no treatment outcome were excluded. Cluster plots of the remaining samples were checked by visual inspection and 38 ambiguous calls and SNPs with MAF less than 0.05 were also excluded from further analysis. A further 8 SNPs were excluded as having poor significance in their Hardy-Weinberg equilibrium i.e., p value<10⁻⁴. This meant that the stage two analysis was carried out in 577 individuals, of which 294 had low response(s) and 261 had high response(s) to therapy (Table 2).

A total of 468 SNPs also passed the quality filters and were selected for stage 2 genotyping.

These 468 SNPs were classified as highly or suggestively associated with the therapeutic response, as described under “statistical analysis” supra. Of these, in stage 2, 40 SNPs achieved the threshold for suggestive evidence of association with treatment response in the replication phase (p≦0.05).

As shown in Table 4, SNPs linked to the IFN-λ1 (IL28B) gene showed moderate-to-strong associations with therapeutic response, including rs8099917 in the 5′-end of the gene, which provided the most highly-significant association (p=9.39×10⁻⁰⁴; OR=1.56; and 95% CI=1.19-2.04). A moderate association was observed for rs12980275 mapping to the 3′-end of IFN-λ3 (p=1.24×10⁻⁴), which had provided a higher significance value in the previous cohort (Table 4). Also shown in Table 4, moderate associations were also observed for the SNPs rs8103142 in exon 2 of the IL28B gene (p=3.83×10⁻⁴) and rs8105790 in the 3′-end of the IL28B gene (p=3.7×10⁻⁴).

Associations with therapeutic outcome were weaker in the stage two cohort for SNPs mapped to other genomic regions, with the exception of rs10018218 and rs1002960, which provided moderate associations.

### c) Merged Data

The Cochran-Armitage trend test (Cochran Biometrics 10, 417-451, 1954; Armitage Biometrics 11, 375-386, 1955) was used to assess association of all SNPs tested in stage one and stage two, and merged P values were determined.

Data presented in Table 4 reveal strong associations with response, reaching genome-wide significance in the overall analysis of the discovery and replication groups, for rs8099917, and rs12980275 flanking the IL28B gene on chromosome 19, with a strong association for rs8109886 in the 5′-end of the IL28B gene.

Data shown in Table 4 also indicate SNPs on other chromosomes that provided moderately-suggestive or highly-suggestive associations with therapeutic outcome, as determined by a merged P value less than 10⁻³. In particular, all of the SNPs on these other chromosomes that were identified in stage one and/or stage two qualified on this basis.

### SNP Genotyping to Determine High Response (HR) and Low Response (LR) Alleles

Conventional methods are used to determine genotypes for the various SNPs listed in Tables 1, 3 and 4, such as, for example a method selected from the following and combinations and variations thereof:

(i) by hybridizing complementary DNA probes to the SNP site in genomic DNA e.g., under high stringency hybridization conditions;
(ii) by dynamic allele-specific hybridization (DASH) employing fluorescently-labelled allele specific oligonucleotides to hybridize to single-stranded biotinylated genomic DNA amplicons bound to a streptavidin column;
(iii) by using a molecular beacon such comprising a sequence of a wild-type allele or a mutant allele;
(iv) by interrogating a SNP microarray using probes comprising the SNP site in several different locations or comprising mismatches to the SNP allele and comparing the signal intensities produced to thereby determine homozygous and heterozygous alleles;
(v) by analyzing restriction fragment length polymorphisms (RFLPs) generated by digestion of genomic DNA using enzymes that distinguish sequence comprising a SNP and resolution of the fragments produced based on their lengths;
(vi) by PCR or other amplification means employing e.g., ARMS primers of different length or differentially labelled and comprising sequences that overlap at the SNP site to thereby amplify the alleles;
(vii) by Invader assay employing e.g., a flap endonuclease (FEN) such as cleavase to digest a tripartite structure comprising genomic DNA and two specific oligonucleotide probes wherein a first probe (the Invader oligonucleotide) is complementary to the 3′ end of the genomic DNA and comprises a mismatched 3′-terminal nucleotide that overlaps the SNP in the target genomic DNA and wherein a second probe (allele-specific oligonucleotide) is complementary to the 5′ end of the target genomic DNA and extends past the 3′ side of the SNP nucleotide and comprises a nucleotide complementary to a SNP allele, such that the tripartite structure forms when the SNP is in the target genomic DNA and cleavase releases the 3′ end of the allele-specific probe from the tripartite structure when the matched allele is present in the allele-specific oligonucleotide;
(vii) by primer extension across the SNP from a probe that is hybridized to the genomic DNA immediately upstream of the SNP nucleotide in the presence of mixes of dNTP/ddNTP mixes each lacking a different ddNTP and sequencing the extension products produced;
(viii) by iPLEX SNP genotyping (Sequenom Inc., San Diego, USA);
(ix) by arrayed primer extension (APEX or APEX-2);
(x) by Infinium assay (Illumina Inc., San Diego, USA) based on primer extension;
(xi) by homogeneous multiplex PCR employing two oligonucleotide primers per SNP to generate amplicons that comprise the alleles in genomic DNA;
(xii) by 5′-nuclease assay employing a thermostable DNA polymerase having 5′-nuclease activity to degrade genomic DNA hybridizing to matched primers but not mismatched primers e.g., performed in real time such as in a Taqman assay format (Applied Biosystems, Carlsbad, USA) and/or in a multiplex assay format;
(xiii) by ligase assay employing matched and mismatched oligonucleotides to interrogate a SNP by hybridizing the probes over the SNP site such that ligation to an upstream or downstream constant oligonucleotide can occur if the probes are identical to the target genomic DNA;
(xiv) by analyzing single strand conformation polymorphisms e.g., as determined by mobility of single-stranded genomic DNA or amplicons produced therefrom;
(xv) by temperature gradient gel electrophoresis (TGGE) or temperature gradient capillary electrophoresis (TGCE) employing target DNA comprising denaturing target DNA comprising the SNP site in the presence of an allele-specific probe comprising a mismatched allele to the target DNA, reannealing the nucleic acids and resolving the products in the presence of a temperature gradient;
(xvi) by denaturing HPLC comprising denaturing target DNA comprising the SNP site in the presence of an allele-specific probe comprising a mismatched allele to the target DNA, reannealing the nucleic acids and resolving the products under reverse-phase HPLC conditions;
(xvii) by high-resolution melting of amplicons;
(xviii) by SNPlex (Applied Biosystems, Carlsbad, USA); and
(xix) by sequencing across SNPs in genomic DNA e.g., employing pyrosequencing.

For example, using conventional methods, HR and LR alleles were determined for SNPs exemplified herein, and these are summarized in Table 3 hereof in the column headed “SNP effect”.

In one particularized example, the rs8099917 SNP was typed by PCR-RFLP using Tsp45I restriction enzyme (New England Biolabs, Beverley, Mass.). Digestions were performed in 10 μl reactions in X1 buffer, 0.4 U enzyme, 5 μl PCR product and Milli Q water at 65° C. for 2 h. Digested products were electrophoresed at 120V for ½ h on a 2% (w/v) TBE gel. Genotype was determined as a 325 by fragment for the T allele, and as fragments of 286 bp and 39 bp for the G allele. Release of a further 214 bp fragment arising from digestion at an internal control Tsp451 site was used to assess completeness of digestion. Data in Table 4 show a very strong association with therapeutic response reaching genome-wide significance for the T allele (and complementary A residue on the opposing DNA strand) of rs8099917 (merged P value=9.25×10⁻⁰⁹, OR=1.86, 95% CI=1.49-2.32). Compared to non-carriers of the high response (HR) allele at rs8099917, heterozygous carriers of the rs8099917 HR allele produced an odds ratio (OR) of 1.64 (95% CI=1.15-2.32) and homozygous carriers produced an OR of 2.39 (95% CI=1.16-4.94).

Data in Table 4 also show a very strong association with therapeutic response reaching genome-wide significance for the A allele (and complementary T residue on the opposing DNA strand) of rs12980275 (merged P value=7.74×10⁻¹⁰).

Data in Table 4 also indicate that the T allele (and complementary A residue on the opposing DNA strand) of rs8103142 in exon 2 of IL28B is associated with a higher response to therapeutic intervention with immunomodulatory compositions i.e., it is the HR allele (p=3.83×10⁻⁴), whereas the C allele (and complementary G residue on the opposing DNA strand) are associated with a lower response i.e., it is the low response (LR) allele.

Data presented in Table 5 show the possible genotypes for two-SNP and three-SNP combinations comprising rs12980275, rs8099917 and rs8103142. These data support the use of combinations of HR alleles and/or LR alleles for the individual SNPs. For example, there is a highly-significant association between therapeutic response and the combination of both HR alleles in rs12980275 and rs8099917 i.e., genotype AA at rs12980275 and genotype TT at rs8099917 (p=6.13×10⁻⁵; OR=2.11; 95% CI=1.46-3.04). Similarly, there is a highly-significant association between therapeutic response and the combination of both HR alleles in rs8103142 and rs8099917 i.e., genotype TT at rs8103142 and genotype TT at rs8099917 (p=4.92×10⁻⁴; OR=2.03; 95% CI=1.36-3.05). The triple homozygotes for HR alleles at these loci are also associated at high significance with therapeutic response i.e., genotype AA at rs12980275 and genotype TT at rs8103142 and genotype TT at rs8099917 (p=6.3×10⁻⁴; OR=2.03; 95% CI=1.36-3.05).

Collectively, the data presented in Tables 4 and 5 suggest that genotypes in 19q13.13 between position 44,420,000 and position 44,440,000 and more specifically between about position 44,423,000 and about position 44,436,000, especially IFN-λ3 (IL-28B) genotypes, are predictive of patient responses to immunomodulatory compositions e.g., an interferon such as IFN-α and/or an agent that modulates Th1/Th2 such as ribavirin.

### Haplotype Analysis for IFN-23 (IL28B)

Haplotypes of SNPs linked to the IFN-λ3 (IL28B) gene were selected using Haplotype Tagger software of the Center for Human Genetic Research of Massachusetts General Hospital and Harvard Medical School, USA, and the Broad Institute, USA, e.g., as described by de Bakker et al., Nature Genetics 37, 1217-1223 (2005). Haplotype Tagger is a tool for the selection and evaluation of SNPs from genotype data, that combines a pairwise tagging method with a multimarker haplotype approach. Genotype data and/or a chromosomal location within which SNPs are mapped are provided as a source for calculation of linkage disequilibrium patterns based on sequence data for the chromosomal region of interest. Haplotype Tagger provides a list of the SNPs and corresponding statistical tests that capture variants of interest. Haplotype Tagger may be implemented in the stand-alone program, Haploview (e.g., version 3.31) of the Broad Institute, USA (e.g., Barrett et al. Bioinformatics 21, 263-265, 2005).

In one example, Haplotype Tagger was employed to fine-map SNPs in the IFN-λ, gene cluster and to tag the common haplotypes in the chromosomal region comprising the IFNλ gene cluster. That analysis identified IFN-λ3 as having a distinct haplotype block for alleles at loci identified herein as being associated with therapeutic response (data not shown).

Pairwise correlation coefficients were determined for IFN-λ3 SNPs and haplotype distributions within the study population were determined. For example, Table 6 shows haplotypes for combinations of the following SNPs:

(a) rs12980275, for which possible alleles are A or G (SEQ ID NO: 64);
(b) rs8105790 for which possible alleles are C or T (SEQ ID NO: 63);
(c) rs8103142 for which possible alleles are C or T (SEQ ID NO: 57);
(d) rs10853727 for which possible alleles are C or T (SEQ ID NO: 26);
(e) rs8109886 for which possible alleles are A or C (SEQ ID NO: 7); and
(f) rs8099917 for which possible alleles are G or T (SEQ ID NO: 4).

The data presented in Table 6 show that the G allele for rs8099917 i.e., the LR allele, tags the haplotype that is most-associated with a low response to therapy (p=3.03×10⁻⁹; OR=2.0; 95% CI=1.58-2.50).

The data presented in Table 6 also show that the HR allele for rs12980275 is linked to HR alleles at rs8105790, rs8103142, rs10853727, rs8109886 and rs8099917 in 45.2% of the test population, and that the LR allele for rs8099917 is associated with LR alleles for rs12980275, rs8105790, rs8103142, rs10853727 and rs8109886 in 25.6% of the test population, suggesting linkage disequilibrium between these alleles. Accordingly, the occurrence of specific alleles linked to IFN-λ3 may predict haplotypes associated with high or low responses to therapy.

### Determining Expression of IFNλ-3 (IL28B)

Total RNA was extracted from whole blood cells of healthy controls according to standard procedures, and used as a template for single-stranded cDNA synthesis using random hexamer primers and Superscript III reverse transcriptase (Invitrogen) according to manufacturer's instruction. RT-PCR was performed employing primers and probes for IFN-λ1 (IL29), IFN-λ2 (IL28A) and IFN-λ3 (IL28B), essentially as described by Mihm et al., C. Lab. Invest. 84, 1148-1159 (2004). The expression levels of the mRNAs were normalized to median expression of glyceraldehyde 3-phosphate dehydrogenase (GAPDH).

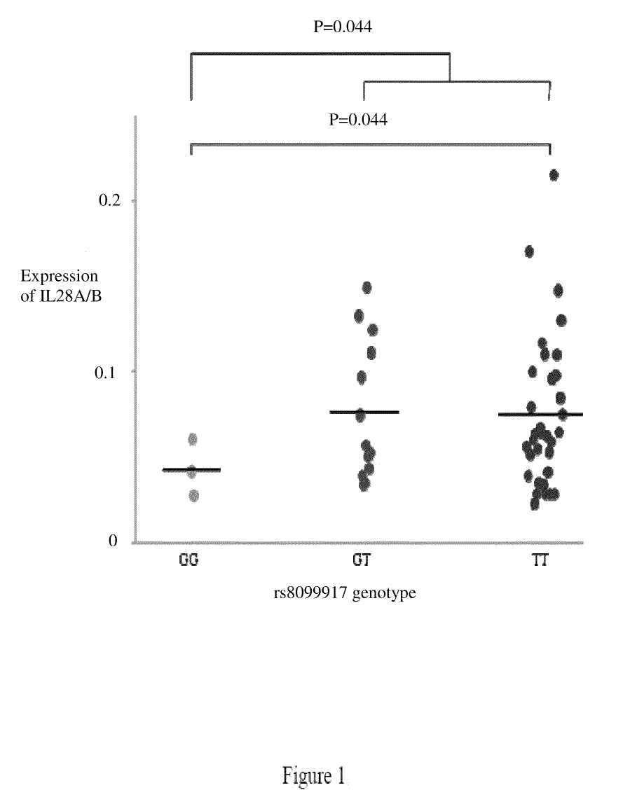

Data in FIG. 1 indicate that expression levels for both IFN-λ2 and IFN-λ3 are higher in carriers of a haplotype having the HR allele for rs8099917 (P<0.04). The haplotype may alter expression in different contexts and with different stimulation e.g., as indicated in Table 1, such as by altering one or more of mRNA splicing, mRNA turnover, mRNA half-life, mRNA stability, affinity of the encoded cytokine for its cognate receptor. Any one or more of these factors may contribute to improved viral clearance for subject having the high response (HR) haplotype. In any event, the data indicate functional significance in the correlation between haplotype and therapeutic efficacy of pegylated interferon-alpha (IFN-α) and ribavirin against HCV.

### Clinical Relevance

Current therapies for HCV-1 employing immunomodulatory compositions such as IFN and ribavirin can produce serious adverse reactions and, in any event, produce low virus clearance in about 50% of infected patients. Accordingly, there is a clear benefit to providing diagnostic and prognostic methods to identify those subjects that are less likely to respond to therapy, thereby avoiding their discomfort. Such diagnostics and prognostic methods also provide a basis for suggesting adjunct or alternative therapies to those patients that are less likely to respond to conventional therapy.

The low response haplotype identified in this study is carried by 70% of northern Europeans, clearly indicating the extent of the problem faced by the pharmaceutical industry for effective therapy of this disease alone

The definition of SNPs, and associations between specific allelic variants at the loci identified in this study, have clear clinical relevance to the diagnosis and treatment using immune response modulators such as interferons, ribavirin and combinations thereof. For example, the identification of a subject carrying a low response (LR) allele at a SNP position identified in this study indicates a reduced likelihood of clearing a virus such as HCV compared to a subject that is a non-carrier of the same allele. Similarly, the identification of a subject carrying a high response (HR) allele at a SNP position identified in this study indicates an enhanced likelihood of clearing virus compared to carriers of the LR allele. For example, 68% of GG homozygotes at the rs1099917 locus fail to clear HCV, whereas only 40% of TT homozygotes at this locus fail to clear HCV. Standard genotyping and haplotyping methods as described herein may be employed to determine the likelihood of a response to therapy in a subject. Thus, the data provided herein provide the means to identify those subjects, including 50% of northern Europeans, who may clear virus on therapy, and those who do not. Using the SNPs identified herein, including the HR haplotype and LR haplotype associations, nearly 90% of subjects capable of having high response(s) to conventional therapy can be identified by their genotype.

The data presented in this study also suggest the broad applicability of a diagnostic/prognostic assay based on IFN-λ3 genotyping and/or haplotyping to the context of virus infections other than HCV. First, the association of IFN-λ3 with viral clearance is consistent with functionality of IFN-λ3 as an antiviral protein, the responsiveness of IFN-λ3 expression to Type 1 interferons such as IFN-α and IFN-β (Li et al, J. Leukocyte Biol., online publication DOI:10.1189/jlb.1208761 (Apr. 30, 2009), and the observation that expression of IFN-λ3 is up-regulated in hepatocytes and PBMCs of HCV-infected patients (Mihm et al., C. Lab. Invest. 84, 1148-1159, 2004). Second, IFN-λ3 is up-regulated by viral infection and by other interferons in hepatocytes and other cells e.g., Siren et al., J. Immunol. 174, 1932-1937 (2005), Ank et al., J. Virol. 80, 4501-4509 (2006), and Doyle et al., Hepatol. 44, 896-906 (2006), and protects against HCV in an in vitro system e.g., Robek et al., J. Virol. 79, 3851-3854 (2005) and Marcello et al., Gastroenterol. 131, 1887-1898 (2006), as well as other RNA viruses in vivo e.g., Ank et al., J. Virol. 80, 4501-4509 (2006) and Ank et al., J. Immunol. 180, 2474-2485 (2008). IFN-λ3 also regulates similar genes to IFN-α via JAK/STAT signalling, however is more specific in its tissue targets. Proceeding on this basis, it is reasonable to conclude that IFN-λ3 provides the basis for diagnosis for those medical indications currently treated using IFNs. It is also reasonable to conclude that IFN-λ3 provides the basis for alternative therapies to those employing other IFNs such as IFN-α, or IFN-λ1, e.g., for those medical indications compatible with IFN-λ3 expression and activity.

Other associations described herein that are not linked to the IFN-λ cluster are also strong indicators of virus clearance, as supported by the available data. The associations with SNPs linked to IL-21R on chromosome 16, caspase-1 (CASP-1) on chromosome 11 and an HLA pseudogene cluster on chromosome 6 are particularly interesting. For example, IL-21 promotes T cell proliferation and viral clearance, and is structurally similar to IFN-λ in terms of exon structure, wherein the alpha helices are encoded by separate exons. Additionally, CASP1 activates IL-1 which then promotes the inflammatory cascade, and inhibits HCV replication in vitro e.g., Zhu et al., J. Virol. 77, 5493-5498 (2003). Proceeding on this basis, it is reasonable to conclude that the other associations described herein provide the basis for diagnosis/prognostic assays in the context of any medical indications currently treated using immunomodulatory compositions other than IFNs and/or ribavirin e.g., compositions comprising IL-1.

TABLE 2
Characteristic for higher responder (HR) and lower-responder (LR) subjects

| | Study | | | | | | | | | | | |
|---|---|---|---|---|---|---|---|---|---|---|---|---|
| | Stage One | | | | Stage Two | | | | | | | |
| | Australian (n = 293) | | | | Berlin (n = 298) | | | | Turin (n = 93) | | | |
| Demographic factors^{a} | HR (131) | | LR (162) | | HR (143) | | LR (155) | | HR (50) | | LR (43) | |
| Age (yr) | 42.0 | (10.0) | 43.9 | (7.0) | 41.3 | (10.4) | 46.9 | (10.1) | 43.1 | (13.0) | 44.3 | (10.1) |
| No. Females (%) | 51 | (38.9) | 35 | (21.6) | 76 | (53.1) | 67 | (43.2) | 27 | (54.0) | 15 | (34.9) |
| No. Males (%) | 80 | (61.1) | 127 | (78.4)^{c} | 67 | (46.9) | 88 | (56.8) | 23 | (46.0) | 28 | (65.1) |
| BMI | 26.9 | (5.0) | 27.5 | (5.1) | 25.2 | (4.5) | 25.9 | (3.9) | 24.1 | (3.4) | 24.6 | (3.5) |
| Viral load (IU/ml) | NS^{b} | | | | NS^{b} | | | | NS^{b} | | | |
| | Study | | | | | | | | | | | |
| | Stage Two | | | | | | | | | | | |
| | UK (n = 91) | | | | Bonn (n = 43) | | | | Australian (n = 32) | | | |
| Demographic factors^{a} | HR (43) | | LR (48) | | HR (13) | | LR (30) | | HR (13) | | LR (19) | |
| Age (yr) | 40.0 | (11.4) | 48.0 | (11.8) | 39.2 | (12.8) | 52.0 | (10.9) | 34.8 | (9.9) | 50.8 | (4.9) |
| No. Females (%) | 14 | (32.6) | 12 | (25.0) | 6 | (46.2) | 10 | (33.3) | 6 | (46.2) | 6 | (31.6) |
| No. Males (%) | 29 | (67.4) | 36 | (75.0) | 7 | (53.8) | 20 | (66.7) | 7 | (53.8) | 13 | (68.4) |
| BMI | 24.6 | (5.9) | 26.4 | (6.4) | 24.3 | (3.5) | 27.3 | (4.6) | 26.7 | (5.3) | 25.7 | (6.3) |

| | | | |
|---|---|---|---|
| Viral load (IU/ml) | NS^{b} | NS^{b} | NS^{b} |

^{a}Unless otherwise specified, mean (s.d.) are presented.
^{b}No significance within each cohort for chi squared comparison of viral load among R vs NR. This methodology was chosen as the viral liters were measured using different kits with different sensitivity between cohorts.
^{c}P < 0.05. No significant difference was observed between stages one and two or between cohorts for age, BMI and viral load.

TABLE 3
Preferred SNPs having alleles associated with efficacy of therapy

| SNP | Chromosome | Position¹ | Location ² | SNP effect | Sequence comprising SNP | SEQ ID NO: |
|---|---|---|---|---|---|---|
| rs10853728 | 19 | 44436986 | IL28A/IL28B | Weak | tgtctcgtaagcagcctgggagatgtgggc[C/G]taagctttggtgaggatgagagtct | 3 |
| | | | intergenic region | | gtctt | |
| rs8099917 | 19 | 44435005 | 5′-end of IL28B | expression level | cctccttttgttttcctttctgtgagcaat[G/T]tcacccaaattggaaccatgctgtatacag | 4 |
| | | | | HR allele = T | cctccttttgttttcctttctgtgagcaatTtcacccaaattggaaccatgctgtatacag | 5 |
| | | | | LR allele = G | cctccttttgttttcctttctgtgagcaatGtcacccaaattggaaccatgctgtatacag | 6 |
| rs8109886 | 19 | 44434603 | 5′-end of IL28B | expression level | ttcttattcatttttccaacaagcatcctg[A/C]cccaggtcgctctgtctgtctcaatcaatc | 9 |
| | | | | HR allele = C | ttcttattcatttttccaacaagcatcctgCcccaggtcgctctgtctgtctcaatcaatc | 10 |
| | | | | LR allele = A | ttcttattcatttttccaacaagcatcctgAcccaggtcgctctgtctgtctcaatcaatc | 11 |
| rs8103142 | 19 | 44426946 | exon 2 of IL28B | missense: K74R | tcctggggaagaggcgggagcggcac[C/T]tgcagtccttcagcagaagcgactct | 66 |
| | | | | mutation in IL28B | | |
| | | | | HR allele = T or A | agagtcgcttctgctgaaggactgcaAgtgccgctcccgcctcttccccagga | 67 |
| | | | | (IL28B-Lvs74) | | |
| | | | | LR allele = C or G | agagtcgcttctgctgaaggactgcaGgtgccgctcccgcctcttccccagga | 69 |
| | | | | (IL28B-Arg74) | | |
| rs8105790 | 19 | 44424341 | 1.75 kb distal | mRNA | ttcccttcctgacatcactccaatgtcctg[C/T]ttctgtggttacatcttccgctaatgatgc | 84 |
| | | | to 3′-end | stability/turnover | | |
| | | | of IL28B | HR allele = T | ttcccttcctgacatcactccaatgtcctgTttctgtggttacatcttccgctaatgatgc | 85 |
| | | | | LR allele = C | ttcccttcctgacatcactccaatgtcctgCttctgtggttacatcttccgctaatgatgc | 86 |
| rs12980275 | 19 | 44423623 | 2.47 kb distal | mRNA | ctgagagaagtcaaattcctagaaac[A/G]gacgtgtctaaatatttgccggggt | 87 |
| | | | to 3′-end | stability/turnover | | |
| | | | of IL28B | HR allele = A | ctgagagaagtcaaattcctagaaacAgacgtgtctaaatatttgccggggt | 88 |
| | | | | LR allele = G | ctgagagaagtcaaattcctagaaacGgacgtgtctaaatatttgccggggt | 89 |
| rs7750468 | 6 | 118183677 | Intergenic | HR allele = A | | |
| | | | to C6orf68 | LR allele = G | taaatgaaatttggaaaacaatccag[A/G]aacaaaatgagaaaatagacaaaga | 90, 91, 92 |
| | | | and SLC35F1 | | | |
| rs2746200 | 6 | 73075162 | RIMS-1 gene | HR allele = C | | |
| | | | intron | LR allele = T | ggagggtcactgtgattcagtgatgc[C/T]caactccctaagagtcttaccaaaa | 93, 94, 95 |
| rs927188 | 6 | 51917576 | PHKD-1 gene | HR allele = A | | |
| | | | intron | LR allele = C | ttgtagaaattgagcaggttgtagat[A/C]taatcacccggtgggttcttcctgc | 96, 97, 98 |
| rs2517861 | 6 | 29929961 | Intergenic to | HR allele = G | | |
| | | | HLA pseudogenes | LR allele = A | tgatatttcttcatgggatggtctcc[A/G]tgatacaatggtaagggaaaacagc | 99, 100, 101 |
| | | | HCP5P10 | | | |
| | | | and MICF | | | |
| rs2025503 | 6 | 23701746 | Intergenic to | HR allele = C | | |
| | | | ALDH5A1 and PRL | LR allele = A | catacactgtacaaagattttcactt[A/C]accaagttggaggactcacttgatc | 102, 103, 104 |
| rs2066911 | 6 | 23656329 | Intergenic to | HR allele = C | | |
| | | | ALDH5A1 and PRL | LR allele = A | catacactgtacaaagattttcactt[A/C]accaagttggaggactcacttgatc | 105. 106, 107 |
| rs10018218 | 4 | 161692769 | Intergenic | HR allele = C | | |
| | | | region | LR allele = T | atgggctcaaatctcatatccttcctccaa[C/T]acgtgttaaaactcaggccctttggtgact | 108, 109, 110 |
| rs1581096 | 4 | 44874493 | Intergenic | HR allele = G | | |
| | | | region | LR allele = A | aaaagagtacaagggatccattttccccat[A/G]tccttactaatacttgctatcatttgtctt | 111, 112, 113 |
| rs1250105 | 4 | 1193265 | Near to | HR allele = G | | |
| | | | CTBP1 | LR allele = A | aaaatcagccaaagcctgcagctaatcctg[A/G]gactggccaggtgacctcacaggagcgcct | 114, 115, 116 |
| rs1939565 | 11 | 930139007 | Near to KIAA1731 | HR allele = A | | |
| | | | and intergenic | LR allele = G | gcaaagcactggcactttattatatttacc[A/G]aaagtacttttggggagagaactaccctat | 117, 118, 119 |
| | | | to FN5 | | | |
| rs568910 | 11 | 104409780 | Intron-2 of | HR allele = G | | |
| | | | CASP-1 | LR allele = T | ctgagtgcaaggggtctgtaggcacttatg[G/T]agttgtaaagtcacatgaagctttaaggtt | 120, 121, 122 |
| rs557905 | 11 | 104403053 | Intron-6 of | HR allele = G | | |
| | | | CASP-1 | LR allele = A | Ccactttgggaatgcacatttagatatttc[A/G]tttccaaatcccaatcactcccctctaccc | 123, 124, 125 |
| rs6806020 | 3 | 54949198 | Intron of | HR allele = T | | |
| | | | CACNA2D3 | LR allele = C | aaaaaaccacacactcaccacattggtgtc[C/T]agtctcaggccacagccccacactcccagt | 126, 127, 128 |
| rs12486361 | 3 | 16430714 | Intron of | HR allele = C | | |
| | | | RTFN-1 gene | LR allele = T | aatagatagaagtgacaaaacctctgcctt[C/T]gtggagctaacaatctaataggaggagaaa | 129, 130, 131 |
| rs10283103 | 8 | 67556167 | intergenic | HR allele = C | | |
| | | | ADHFE1 and | LR allele = T | agttctttattaataagtcacagcatcctg[C/T]aaggaagaaattgtgcatcagctgccaagc | 132, 133, 134 |
| | | | MGC33510 | | | |
| rs2114487 | 8 | 67420305 | Intergenic | HR allele = C | | |
| | | | region RRS1 | LR allele = T | aggacactggaaaagggatagaaacagatt[C/T]tcccccggggccttcagaactgaaagtagt | 135, 136, 137 |
| | | | and CRH | | | |
| rs7196702 | 16 | 77341734 | Intron of | HR allele = A | | |
| | | | WWOX gene | LR allele = G | ttcatagctgtcttgcccctcctgtggtct[A/G]taagaatgggaccaggactcctagttgtga | 138, 139, 140 |
| rs3093390 | 16 | 27370949 | Near to IL21R | HR allele = T | | |
| | | | and intergenic | LR allele = C | gttgggaagagatatgcacaatctgccctc[C/T]tggctggtatgagtgagtcccagctcaccg | 141, 142, 143 |
| | | | to GFT3C1 | | | |
| rs7512595 | 1 | 27729758 | Intergenic | HR allele = G | | |
| | | | region WASF2 | LR allele = A | agaccaaatgcattaatacatatgcaaagc[A/G]tttggaacagctggcatatataagtgccat | 144, 145, 146 |
| | | | and ADHC1 | | | |
| rs1002960 | 9 | 88029735 | Intergenic | HR allele = A | | |
| | | | region | LR allele = C | cggcccttgtctgcgtacccctagacttct[A/C]attatgtaagaaaaataaccactatttggt | 147, 148, 149 |
| rs1931704 | 10 | 129229799 | Near to NPS | HR allele = G | | |
| | | | and intergenic | LR allele = A | taggaggaaacgtgtgaagagggcttgggt[A/G]actctaagacagttacctcatgacaaagaa | 150, 151, 152 |
| | | | to NPS | | | |
| | | | and DOCK1 | | | |
| rs66616 | 14 | 58286251 | Intergenic | HR allele = G | | |
| | | | to DACT1 and | LR allele = A | gaaaaacaagaaagctggtttctttgattt[A/G]acagacaatgtatagaccatttgggcactg | 153, 154, 155 |
| | | | LOC729646 | | | |
| rs4402825 | 20 | 45765623 | Intron-3 of | HR allele = T | | |
| | | | SULF2 gene | LR allele = C | gtttgtggatcccttggattctgtctgcta[C/T]acagcaaccagaatggctaacattaaagaa | 156, 157, 158 |

¹Chromosome positions are derived from Hapmap project data release 27.
²Gene locations were obtained by scanning ±100 kb from the associated SNP
HR allele, Allele associated with higher response to therapy.
LR allele, Allele associated with lower response or no response to therapy.

TABLE 4
SNP and genotype associations with efficacy of therapy

| | Stage one | Stage 2 | Merged | OR | | Tested | Genotype | OR |
|---|---|---|---|---|---|---|---|---|
| SNP | p value¹ | p value¹ | p value² | (95% C.I.)³ | Possible genotypes | genotype | P value | (95% C.I.) |
| Chromosome 19: | | | | | | | | |
| rs4803224 | 5.50 × 10⁻⁰² | 0.2 | 0.77 | | C/C C/G G/G | | | |
| rs12980602 | 1.08 × 10⁻⁰² | 2.66 × 10⁻⁰² | 1.02 × 10⁻⁰³ | | C/C C/T T/T | | | |
| rs10853728 | 2.67 × 10⁻⁰³ | 0.97 | 7.42 × 10⁻⁰² | | C/C C/G G/G | | | |
| rs8099917 | 7.06 × 10⁻⁰⁸ | 9.39 × 10⁻⁰⁴ | 9.25 × 10⁻⁰⁹ | 1.86 (1.49-2.32) | G/G G/T T/T | | | |
| | | | | | | G/G | 0.066 | |
| | | | | | | G/T | | |
| | | | | | | T/T | 0.0015 | 1.72 (1.23-2.41) |
| rs8113007 | | | | | A/A A/T T/T | | | |
| rs8109889 | | | | | C/C C/T T/T | | | |
| rs8109886 | 1.29 × 10⁻⁰⁴ | 3.44 × 10⁻⁰² | 1.27 × 10⁻⁰⁴ | | A/A A/C C/C | | | |
| rs61599059 | | | | | */* */CT CT/CT | | | |
| rs34567744 | | | | | */* */CT CT/CT | | | |
| rs10642510 | | | | | */* */CT */TC CT/TC CT/CT TC/TC | | | |
| rs 10643535 | | | | | **/** **/CT CT/CT | | | |
| rs34593676 | | | | | **/** **/TC TC/TC | | | |
| rs 25122122 | | | | | */* */T T/T | | | |
| rs35407108 | | | | | */* */A A/A | | | |
| rs59211796 | | | | | A/A A/G G/G | | | |
| rs62120529 | | | | | A/A A/G G/G | | | |
| rs62120528 | | | | | A/A A/C C/C | | | |
| rs12983038 | | | | | A/A A/G G/G | | | |
| rs10853727 | 0.72 | 0.22 | 0.43 | | C/C C/T T/T | | | |
| rs7254424 | | | | | A/A A/G G/G | | | |
| rs1549928 | | | | | A/A A/G G/G | | | |
| rs34347451 | | | | | */* */A A/A | | | |
| rs35814928 | | | | | */* */A A/A | | | |
| rs4803222 | | | | | C/C C/G G/G | | | |
| rs11322783 | | | | | */* */T T/T | | | |
| rs4803221 | | | | | C/C C/G G/G | | | |
| rs12979860 | | | | | C/C C/T T/T | | | |
| rs12971396 | | | | | C/C C/G G/G | | | |
| rs11672932 | | | | | C/C C/G G/G | | | |
| rs11882871 | | | | | A/A A/G G/G | | | |
| rs56215543 | | | | | A/A A/G G/G | | | |
| rs12979731 | | | | | C/C C/T T/T | | | |
| rs2020358 | | | | | G/G G/T T/T | | | |
| rs34853289 | | | | | C/C C/T T/T | | | |
| rs8107030 | | | | | A/A A/G G/G | | | |
| rs41537748 | | | | | C/C C/T T/T | | | |
| rs59702201 | | | | | */* */ATAT ATAT/ATAT | | | |
| rs2596806 | | | | | C/C C/G G/G | | | |
| rs2569377 | | | | | A/A A/G G/G | | | |
| rs4803219 | | | | | C/C C/T T/T | | | |
| rs28416813 | | | | | C/C C/G G/G | | | |
| rs630388 | | | | | A/A A/G G/G | | | |
| rs629976 | | | | | A/A A/G G/G | | | |
| rs629976 | | | | | | A/A | | |
| rs629976 | | | | | | G/G | | |
| rs629008 | | | | | A/A A/G G/G | | | |
| rs628973 | | | | | A/A A/T T/T | | | |
| rs8103142 | — | 3.83 × 10⁻⁰⁴ | — | | C/C C/T T/T | | | |
| | | | | | | C/C | 0.033 | 0.62 (0.39-0.96) |
| | | | | | | C/T | | |
| | | | | | | T/T | 0.000492 | 2.03 (1.36-3.05) |
| rs8102358 | | | | | A/A A/G G/G | | | |
| rs11881222 | | | | | A/A A/G G/G | | | |
| rs61735713 | | | | | C/C C/T T/T | | | |
| rs61735713 | | | | | | C/C | | |
| rs61735713 | | | | | | T/T | | |
| rs62120527 | | | | | C/C C/T T/T | | | |
| rs62120527 | | | | | | C/C | | |
| rs62120527 | | | | | | T/T | | |
| rs4803217 | | | | | A/A A/C C/C | | | |
| rs8105790 | — | 3.70 × 10⁻⁰⁴ | — | | C/C C/T T/T | | | |
| rs12980275 | 4.81 × 10⁻⁰⁸ | 1.24 × 10⁻⁰⁴ | 7.74 × 10⁻¹⁰ | | A/A A/G G/G | | | |
| | | | | | | A/A | 0.0000908 | 2.06 (1.43-2.97) |
| | | | | | | A/G | | |
| | | | | | | G/G | 0.036 | 0.61 (0.39-0.97) |
| Chromosome 6: | | | | | | | | |
| rs7750468 | 2.34 × 10⁻⁵ | 0.117 | <1.0 × 10⁻⁴ | 1.95 (1.37-2.78) | A/A A/G G/G | | | |
| rs2746200 | 3.0 × 10⁻⁴ | 0.1309 | 7.0 × 10⁻⁴ | 1.39 (1.14-1.68) | C/C C/T T/T | | | |
| rs927188 | 4.0 × 10⁻⁴ | 0.0671 | 5.62 × 10⁻⁴ | 1.43 (1.17-1.75) | T/T T/G G/G | | | |
| rs2517861 | 1.1 × 10⁻³ | 0.0658 | 8.32 × 10⁻⁴ | 1.52 (1.2-1.92) | C/C C/T T/T | | | |
| rs2025503 | 1.0 × 10⁻⁴ | 0.0151 | <1.0 × 10⁻⁴ | 1.66 (1.30-2.10) | C/C C/A A/A | | | |
| rs2066911 | 1.13 × 10⁻⁵ | 0.1245 | 1.3 × 10⁻⁴ | 1.53 (1.23-1.91) | G/G G/A A/A | | | |
| Chromosome 4: | | | | | | | | |
| rs10018218 | 1.0 × 10⁻⁴ | 4.9 × 10⁻³ | <1.0 × 10⁻⁴ | 1.79 (1.39-2.31) | C/C C/T T/T | | | |
| rs1581096 | 1.2 × 10⁻³ | 0.0365 | 9.0 × 10⁻⁴ | 2.01 (1.33-3.02) | C/C C/T T/T | | | |
| rs1250105 | 1.2 × 10⁻³ | 0.054 | 7.54 × 10⁻⁴ | 1.4 (1.15-1.70) | C/C C/T T/T | | | |
| Chromosome 11: | | | | | | | | |
| rs1939565 | 5.0 × 10⁻⁴ | 0.0314 | 2.08 × 10⁻⁴ | 1.44 (1.19-1.74) | T/T T/C C/C | | | |
| rs568910 | 6.6 × 10⁻³ | 0.0213 | 4.85 × 10⁻⁴ | 1.58 (1.22-2.05) | C/C C/A A/A | | | |
| rs557905 | 6.6 × 10⁻³ | 0.0147 | 2.95 × 10⁻⁴ | 1.60 (1.23-2.08) | C/C C/T T/T | | | |
| Chromosome 3: | | | | | | | | |
| rs6806020 | 3.78 × 10⁻⁵ | 0.0553 | <1.0 × 10⁻⁴ | 1.52 (1.23-1.87) | T/T T/C C/C | | | |
| rs12486361 | 3.0 × 10⁻⁴ | 0.0495 | 2.29 × 10⁻⁴ | 1.43 (1.18-1.74) | C/C C/T T/T | | | |
| Chromosome 8: | | | | | | | | |
| rs10283103 | 4.0 × 10⁻⁴ | 0.0875 | 5.16 × 10⁻⁴ | 1.53 (1.20-1.94) | C/C C/T T/T | | | |
| rs2114487 | 9.51 × 10⁻⁵ | 0.2689 | 9.8 × 10⁻⁴ | 1.52 (1.19-1.94) | C/C C/T T/T | | | |
| Chromosome 16: | | | | | | | | |
| rs7196702 | 7.0 × 10⁻⁴ | 0.0998 | 8.37 × 10⁻⁴ | 1.69 (1.24-2.29) | A/A A/G G/G | | | |
| rs3093390 | 8.0 × 10⁻⁴ | 0.0331 | 3.29 × 10⁻⁴ | 1.53 (1.22-1.92) | T/T T/C C/C | | | |
| Chromosomes 1, | | | | | | | | |
| 9, 10, 14 and 20: | | | | | | | | |
| rs7512595 | 4.0 × 10⁻⁴ | 0.0975 | 6.2 10⁻⁴ | 1.77 (1.27-2.47) | G/G G/A A/A | | | |
| rs1002960 | 2.0 × 10⁻⁴ | 9.7 × 10⁻³ | <1.0 × 10⁻⁴ | 1.7 (1.33-2.16) | A/A A/C C/C | | | |
| rs1931704 | 1.46 × 10⁻⁷ | 0.2231 | <1.0 × 10⁻⁴ | 1.56 (1.25-1.94) | G/G G/A G/G | | | |
| rs66616 | 6.0 × 10⁻⁴ | 0.0752 | 8.83 × 10⁻⁴ | 1.68 (1.24-2.26) | C/C C/T T/T | | | |
| rs4402825 | 3.0 × 10⁻⁴ | 0.0376 | 1.8 × 10⁻⁴ | 1.59 (1.25-2.02) | T/T T/C C/C | | | |

¹Stage one and stage two p-values are based on allelic comparisons obtained from Haploview.
²Merged p-values are based on cochrane-armitage trend test results.
³Odds ratio (OR) and 95% confidence interval (95% C.I.) are based on allelic distributions of SNPs for the combined cohort.

TABLE 5
Associations of chromosome 19 SNP combinations with efficacy of therapy

| | | Tested | | |
|---|---|---|---|---|
| SNP | | genotype | Genotype combination | |
| combination | Possible genotype combinations | combination | P value | OR (95% C.I.) |
| rs 12980275 | GG GG, GG TG, AG GG, GG TT, | AA TT | 0.0000613 | 2.11 (1.46-3.04) |
| rs 8099917 | AG TG, AG TT, AA TG, AA TT | (HR HR) | | |
| | | GG GG | 0.042 | 0.47 (0.23-0.99) |
| | | (LR LR) | | |
| rs8103142 | CC GG, CC TG, CC TT, | TT TT | 0.000492 | 2.03 (1.36-3.05) |
| rs 8099917 | CT TG, CT TT, TT TT | (HR HR) | | |
| | | CC GG | 0.077 | |
| | | (LR LR) | | |
| rs 12980275 | AA CC GG, AA CC GT, AA CC TT, | AA TT TT | 0.00063 | 2.03 (1.36-3.05) |
| | AA CT GG, AA CT GT, AA CT TT, | (HR HR HR) | | |
| rs8103142 | AA TT GG, AA TT GT, AA TT TT, | | | |
| rs 8099917 | AG CC GG, AG CC GT, AG CC TT, | | | |
| | AG CT GG, AG CT GT, AG CT TT, | GG CC GG | 0.049 | 0.49 (0.23-1.01) |
| | AG TT GG, AG TT GT, AG TT TT, | (LR LR LR) | | |
| | GG CC GG, GG CC GT, GG CC TT, | | | |
| | GG CT GG, GG CT GT, GG CT TT, | | | |
| | GG TT GG, GG TT GT, GG TT TT, | | | |

| SNP | |
|---|---|
| combination | Sequence comprising SNP (SEQ ID NO:) |
| rs 12980275 | ctgagagaagtcaaattcctagaaacAgacgtgtctaaatatttgccggggt (SEQ ID NO: 88) |
| rs 8099917 | cctccttttgttttcctttctgtgagcaatTtcacccaaattggaaccatgctgtatacag (SEQ ID NO: 5) |
| | ctgagagaagtcaaattcctagaaacGgacgtgtctaaatatttgccggggt (SEQ ID NO: 89) |
| | cctccttttgttttcctttctgtgagcaatGtcacccaaattggaaccatgctgtatacag (SEQ ID NO: 6) |
| rs8103142 | tcctggggaagaggcgggagcggcacTtgcagtccttcagcagaagcgactct |
| rs 8099917 | (reverse complement of SEQ ID NO: 67) |
| | cctccttttgttttcctttctgtgagcaatTtcacccaaattggaaccatgctgtatacag (SEQ ID NO: 5) |
| | tcctggggaagaggcgggagcggcacCtgcagtccttcagcagaagcgactct |
| | (reverse complement of SEQ ID NO: 68) |
| | cctccttttgttttcctttctgtgagcaatGtcacccaaattggaaccatgctgtatacag (SEQ ID NO: 6) |
| rs 12980275 | ctgagagaagtcaaattcctagaaacAgacgtgtctaaatatttgccggggt (SEQ ID NO: 88) |
| | tcctggggaagaggcgggagcggcacTtgcagtccttcagcagaagcgactct |
| rs8103142 | (reverse complement of SEQ ID NO: 67) |
| rs 8099917 | cctccttttgttttcctttctgtgagcaatTtcacccaaattggaaccatgctgtatacag (SEQ ID NO: 5) |
| | ctgagagaagtcaaattcctagaaacGgacgtgtctaaatatttgccggggt (SEQ ID NO: 89) |
| | tcctggggaagaggcgggagcggcacCtgcagtccttcagcagaagcgactct |
| | (reverse complement of SEQ ID NO: 68) |
| | cctccttttgttttcctttctgtgagcaatGtcacccaaattggaaccatgctgtatacag (SEQ ID NO: 6) |

HR, genotype homozygous for HR alleles at designated locus associated with higher response to therapy.
LR, genotype homozygous for LR alleles at designated locus associated with lower response to therapy.

TABLE 6
Haplotype effects for six chromosome 19 SNP
allele combinations on efficacy of therapy

| | Average | | | | |
|---|---|---|---|---|---|
| SNP haplotype | frequency | Frequency in | Frequency in | Haplotype | |
| for alleles (a)-(f)¹ | in cohort (%) | HR³ (%) | LR⁴ (%) | p value | OR (95% CI)² |
| A T T T C T | 45.2 | 49.4 | 41.5 | 1.2 × 10⁻⁰³ | 1.37 (1.13-1.67) |
| G C C T A G | 25.6 | 18.8 | 31.5 | 3.03 × 10⁻⁰⁹ | 2.0 (1.58-2.50) |
| G T C C A T | 11.2 | 10.7 | 11.7 | 0.52 | 1.11 (0.81-1.50) |
| A T T T A T | 10.5 | 13.0 | 8.3 | 1.9 × 10⁻⁰³ | 1.64 (1.20-2.25) |
| A T C T A T | 2.2 | 2.4 | 2.0 | 0.51 | 1.23 (0.64-2.36) |
| G C C T A T | 1.8 | 1.4 | 2.1 | 0.27 | 1.5 (0.71-3.18) |
| G T T T C T | 1.1 | 1.4 | 0.9 | 0.42 | 0.63 (0.25-1.56) |

¹Haplotypes are shown in order from left to right for combinations of the following SNP: (a) rs12980275 for which possible alleles are A or G (SEQ ID NO: 64); (b) rs8105790 for which possible alleles are C or T (SEQ ID NO: 63); (c) rs8103142 for which possible alleles are C or T (SEQ ID NO: 57); (d) rs10853727 for which possible alleles are C or T (SEQ ID NO: 26); (e) rs8109886 for which possible alleles are A or C (SEQ ID NO: 7); and (f) rs8099917 for which possible alleles are G or T (SEQ ID NO: 4).
²Odds ratios of each haplotype were calculated as carriage vs non-carriage of the haplotype.
³HR, subjects having a higher response to therapy.
⁴LR, subjects having a lower response to therapy.

1. A method for accurately determining the likelihood that a subject will respond to treatment with an immunomodulatory composition, said method comprising detecting one or more markers in a sample from the subject, wherein at least one marker is linked to a single nucleotide polymorphism (SNP) set forth in Table 1 or comprises a SNP set forth in Table 1 or is encoded by nucleic acid comprising a SNP set forth in Table 1 or linked to a SNP set forth in Table 1, and, wherein detection of said one or more markers is indicative of the likely response of the subject to treatment with said composition.

2. The method according to claim 1, wherein at least one marker is linked to a SNP set forth in Table 3 or comprises a SNP set forth in Table 3 or is encoded by nucleic acid comprising a SNP set forth in Table 3 or linked to a SNP set forth in Table 3.

3. The method according to claim 1, wherein at least one marker is linked to a SNP set forth in Table 4 or 5 or comprises a SNP set forth in Table 4 or 5 or is encoded by nucleic acid comprising a SNP set forth in Table 4 or 5 or linked to a SNP set forth in Table 4 or 5.

4-8. (canceled)

9. The method according to claim 1, wherein at least one marker is linked to an IFN-λ3 gene or is contained within an IFN-λ3 gene or comprises an IFN-λ3 gene or is encoded by an IFN-λ3 gene.

10. (canceled)

11. The method according to claim 9, wherein at least one marker comprises an allele associated with a response to treatment with the immunomodulatory composition, wherein said allele is contained within a sequence selected from the group consisting of:
(i) a sequence set forth in any one of SEQ ID NOs: 5, 10, 67, 85 and 88; and
(ii) a sequence complementary to a sequence at (i),
wherein detection of said at least one marker is indicative of a response of the subject to treatment with said composition.

12. The method according to claim 9, wherein at least one marker comprises an allele associated with a low response or non-response to treatment with the immunomodulatory composition, wherein said allele is contained within a sequence selected from the group consisting of:
(i) a sequence set forth in any one of SEQ ID NOs: 6, 11, 69, 86 and 89; and
(ii) a sequence complementary to a sequence at (i),
wherein detection of said at least one marker is indicative of a low response or non-response to treatment of the subject to treatment with said composition.

13. The method according to claim 9, wherein at least one marker is encoded by a sequence comprising a polymorphic nucleotide, wherein said sequence is selected from the group consisting of: SEQ ID NOs: 60, 62, 67, 69, 74, 76, 79 and 81.

14. The method according to claim 9, wherein at least one marker comprises an amino acid sequence comprising a polymorphic amino acid, wherein said sequence is selected from the group consisting of: SEQ ID NOs: 61, 63, 68, 70, 75, 77, 80 and 82.

15. (canceled)

16. (canceled)

17. The method according to claim 9 comprising detecting a plurality of the markers.

18. The method according to claim 17 comprising detecting two of the markers.

19. The method according to claim 17 comprising detecting three of the markers.

20. The method according to claim 17 comprising detecting six of the markers.

21. The method according to claim 9 comprising detecting a haplotype comprising a plurality of the markers.

22. The method according to claim 21, wherein the haplotype comprises an allele at rs8099917.

23. The method according to claim 22, wherein the haplotype comprises an allele at each of rs12980275, rs8105790, rs8103142, rs10853727, rs8109886 and rs8099917, and, wherein detection of a haplotype comprising said allele is indicative of a low response or non-response to treatment of the subject to treatment with said composition.

24. The method according to claim 22, wherein the allele comprises a C or G nucleotide at rs8099917 and, wherein detection of a haplotype comprising said allele is indicative of a low response or non-response to treatment of the subject to treatment with said composition.

25. The method according to claim 22, wherein the haplotype comprises an allele at each of rs12980275, rs8105790, rs8103142, rs10853727, rs8109886 and rs8099917, and, wherein detection of a haplotype comprising said allele is indicative of a response to treatment of the subject to treatment with said composition.

26. The method according to claim 9 comprising detecting a modified level of expression of one or more of the genes in a sample from the subject, wherein said modified expression is indicative of a response of the subject to treatment with said composition.

27. The method according to claim 26, wherein expression of the gene is increased.

28. The method according to claim 9 comprising detecting a modified level of expression of one or more of the genes, wherein said modified expression is indicative of a low response or non-response to treatment of the subject to treatment with said composition.

29. The method according to claim 28, wherein expression of the gene(s) is reduced.

30. The method according to claim 26 comprising detecting a modified level of at least one expression product of the gene(s) by nucleic acid-based assay or antigen-based assay.

31. The method according to claim 26 comprising performing an amplification reaction to detect an mRNA transcript of the gene(s) in a sample from the subject.

32. The method according to claim 26 comprising contacting a biological sample derived from a subject with an antibody or ligand capable of specifically binding to an allelic variant of a protein encoded by the gene(s) said marker for a time and under conditions sufficient for complex to form and then detecting the complex.

33-35. (canceled)

36. The method according to claim 9, wherein the sample is selected from the group consisting of whole blood, serum, plasma, peripheral blood mononuclear cells (PBMC), a buffy coat fraction, saliva, urine, a buccal cell, liver biopsy and a skin cell.

37-39. (canceled)

40. The method according to claim 9, wherein detection of said one or more markers is indicative of a response selected from the group consisting of:
(i) a response comprising enhanced clearance of a virus or a reduction in virus titer or a change in other health characteristic of the subject related to reduced virus titer or enhanced clearance;
(ii) a response comprising recovery or remission from cancer or reduced growth of a tumor or pre-cancerous lesion;
(iii) a change in Th1 cell number, Th2 cell number or Th1/Th2 cell balance or a change in other health characteristic of the subject indicative of recovery from a Th1-mediated or Th2-mediated disease; and
(iv) a combination of two or all of (i) to (iii).

41. The method according to claim 9, wherein detection of said one or more markers is indicative of a low response or non-response selected from the group consisting of:
(i) a failure to clear of a virus/bacteria or to reduce virus titer or bacterial count change in other health characteristic of the subject related to said failure;
(ii) a failure to recover or enter remission from cancer or to reduce growth of a tumor or pre-cancerous lesion;
(iii) no significant change in Th1 cell number, Th2 cell number or Th1/Th2 cell balance or health characteristic of the subject that would indicate recovery from a Th1-mediated or Th2-mediated disease; and
(iv) a combination of two or all of (i) to (iii).

42. The method according to claim 9, wherein the subject is Caucasian.

43. The method according to claim 9, wherein the subject is African or Asian.

44. The method according to claim 1, wherein the immunomodulatory composition comprises one or more IFNs and/or one or more derivatives of said one or more of said IFNs.

45. The method according to claim 44, wherein the composition comprises one or more IFNs selected from IFN-α, IFN-β, IFN-ω, IFN-γ, IFN-λ1, IFN-λ2 and IFN-λ3 and/or one or more derivatives of any one or more of said IFNs.

46. The method according to claim 1, wherein the immunomodulatory composition comprises one or more guanosine analogs and/or one or more derivatives of said one or more of said guanosine analogs.

47. The method according to claim 46, wherein the composition comprises one or more guanosine analogs selected from ribavirin, viramidine, 7-benzyl-8-bromoguanine, 9-benzyl-8-bromoguanine, and CpG-containing oligonucleotide(s), and derivative(s), salt(s), solvate(s) and hydrate(s) thereof.

48. The method according to claim 9, wherein the immunomodulatory composition comprises IFN-α and ribavirin.

49. The method according to claim 48, wherein the IFN is pegylated IFN.

50-60. (canceled)

61. A process for accurately determining the likelihood that a subject will respond to treatment of HCV infection with an immunomodulatory composition, said process comprising performing the method according to claim 9 to thereby detect one or more markers indicative of the likely response of the subject to treatment with said composition, and determining a response for the subject selected from the group consisting of:
(i) a response comprising enhanced clearance of HCV or a reduction in HCV titer or a change in other health characteristic of the subject related to reduced virus titer or enhanced clearance, wherein said response is indicative of a response to treatment; and
(ii) a failure to clear HCV or to reduce HCV titer or a change in a health characteristic of the subject related to said failure, wherein said response is indicative of a low response or no response to treatment.

62. The process according to claim 61, wherein the immunomodulatory composition comprises one or more IFNs and/or one or more derivatives of said one or more of said IFNs.

63. The process according to claim 62, wherein the composition comprises one or more IFNs selected from IFN-α, IFN-β, IFN-ω, IFN-γ, IFN-λ1, IFN-λ2 and IFN-λ3 and/or one or more derivatives of any one or more of said IFNs.

64. The process according to claim 62, wherein an IFN is pegylated IFN.

65. The process according to claim 61, wherein the immunomodulatory composition comprises one or more guanosine analogs and/or one or more derivatives of said one or more of said guanosine analogs.

66. The process according to claim 65, wherein the composition comprises one or more guanosine analogs selected from ribavirin, viramidine, 7-benzyl-8-bromoguanine, 9-benzyl-8-bromoguanine, and CpG-containing oligonucleotide(s), and derivative(s), salt(s), solvate(s) and hydrate(s) thereof.

67. A process for accurately determining the likelihood that a subject will respond to treatment of HCV infection with an immunomodulatory composition comprising an IFN or a derivative thereof and ribavirin or a derivative thereof, said process comprising performing the method according to claim 9 to thereby detect one or more markers indicative of the likely response of the subject to treatment with said composition, and determining a response for the subject selected from the group consisting of:
(i) a response comprising enhanced clearance of HCV or a reduction in HCV titer or a change in other health characteristic of the subject related to reduced virus titer or enhanced clearance, wherein said response is indicative of a response to treatment; and
(ii) a failure to clear HCV or to reduce HCV titer or a change in a health characteristic of the subject related to said failure, wherein said response is indicative of a low response or no response to treatment.

68-70. (canceled)

71. A process comprising:
(i) performing a method according to claim 9; and
(ii) administering or recommending an immunomodulatory composition to a subject.

72. A process comprising:
(i) obtaining results of a method according to claim 9; and
(ii) administering or recommending an immunomodulatory composition to a subject.

73-85. (canceled)

86. A process for determining a predisposition in a subject to a chronic HCV infection, said process comprising performing the method according to claim 9 to thereby identify a subject likely to not respond to treatment with an immunomodulatory composition or likely to provide a low response to treatment, and determining that the subject has a predisposition to chronic HCV infection.

87. A method of treatment of HCV-infection in a subject, said method comprising administering or recommending to the subject an immunomodulatory composition comprising an IFN-λ2 or a derivative thereof and/or an IFN-λ3 or a derivative thereof to a subject in need thereof.

88. The method according to claim 87, wherein administration of the immunomodulatory composition is for a time and under conditions sufficient to enhance viral clearance or reduce virus titer in the subject.

89. The method according to claim 88, wherein the derivative is pegylated IFN-λ2 and/or pegylated IFN-λ3 and/or albuminated IFN-λ2 and/or albuminated IFN-λ3.

90. The method according to claim 87 further comprising administering or recommending administration of a guanosine analog to the subject.

91-98. (canceled)

99. The method according to claim 28 comprising detecting a modified level of at least one expression product of the gene(s) by nucleic acid-based assay or antigen-based assay.

100. The method according to claim 28 comprising performing an amplification reaction to detect an mRNA transcript of the gene(s) in a sample from the subject.

101. The method according to claim 28 comprising contacting a biological sample derived from a subject with an antibody or ligand capable of specifically binding to an allelic variant of a protein encoded by the gene(s) said marker for a time and under conditions sufficient for complex to form and then detecting the complex.


SEQUENCE LISTING (158 sequences)

SEQ ID NO: 1 - DNA, 61 bp, artificial
  Sequence comprising SNP rs4803224
aaaaaaaaatagaagaattatctgggcatgstggtgggtgcctgcagctccagctgcttag

SEQ ID NO: 2 - DNA, 61 bp, artificial
  Sequence comprising SNP rs1290602
atattcatataacaatatgaaagccagagayagctcgtctgagacacagatgaacaaaaac

SEQ ID NO: 3 - DNA, 61 bp, artificial
  Sequence comprising SNP rs10853728
tgtctcgtaagcagcctgggagatgtgggcstaagctttggtgaggatgagagtctgtctt

SEQ ID NO: 4 - DNA, 61 bp, artificial
  Sequence comprising SNP rs8099917
cctccttttgttttcctttctgtgagcaatktcacccaaattggaaccatgctgtatacag

SEQ ID NO: 5 - DNA, 61 bp, artificial
  Sequence comprising HR allele of SNP rs8099917
cctccttttgttttcctttctgtgagcaatttcacccaaattggaaccatgctgtatacag

SEQ ID NO: 6 - DNA, 61 bp, artificial
  Sequence comprising LR allele of SNP rs8099917
cctccttttgttttcctttctgtgagcaatgtcacccaaattggaaccatgctgtatacag

SEQ ID NO: 7 - DNA, 61 bp, artificial
  Sequence comprising SNP rs8113007
ttaaagtaagtcttgtatttcacctcctggwggtaaatattttttaacaatttgtcactgt

SEQ ID NO: 8 - DNA, 61 bp, artificial
  Sequence comprising SNP rs8109889
catttttccaacaagcatcctgccccaggtygctctgtctgtctcaatcaatctctttttg

SEQ ID NO: 9 - DNA, 61 bp, artificial
  Sequence comprising SNP rs8109886
ttcttattcatttttccaacaagcatcctgmcccaggtcgctctgtctgtctcaatcaatc

SEQ ID NO: 10 - DNA, 61 bp, artificial
  Sequence comprising HR allele of SNP rs8109886
ttcttattcatttttccaacaagcatcctgccccaggtcgctctgtctgtctcaatcaatc

SEQ ID NO: 11 - DNA, 61 bp, artificial
  Sequence comprising LR allele of SNP rs8109886
ttcttattcatttttccaacaagcatcctgacccaggtcgctctgtctgtctcaatcaatc

SEQ ID NO: 12 - DNA, 60 bp, artificial
  Sequence comprising delta(CT) allele of SNP rs61599059
gtcttgctttctctttctctctctctctctgttcctgtctctgtctctggcgtgactcca

SEQ ID NO: 13 - DNA, 62 bp, artificial
  Sequence comprising “CT” allele of SNP rs61599059
gtcttgctttctctttctctctctctctctctgttcctgtctctgtctctggcgtgactcca

SEQ ID NO: 14 - DNA, 60 bp, artificial
  Sequence comprising delta(CT) allele of SNP rs34567744
tgtgtcttgctttctctttctctctctctctctgttcctgtctctgtctctggcgtgact

SEQ ID NO: 15 - DNA, 62 bp, artificial
  Sequence comprising “CT” allele of SNP rs34567744
tgtgtcttgctttctctttctctctctctccttctgttcctgtctctgtctctggcgtgact

SEQ ID NO: 16 - DNA, 54 bp, artificial
  Sequence comprising delta(CT)/delta(TC) allele of SNP rs10642510
tgtcttgctttctctttctctctctctctctgttcctgtctctgtctctggcgt

SEQ ID NO: 17 - DNA, 56 bp, artificial
  Sequence comprising “CT” allele of SNP rs10642510
tgtcttgctttctctttctctctctctctctctgttcctgtctctgtctctggcgt

SEQ ID NO: 18 - DNA, 56 bp, artificial
  Sequence comprising “TC” allele of SNP rs10642510
tgtcttgctttctctttctctctctcttcctctgttcctgtctctgtctctggcgt

SEQ ID NO: 19 - DNA, 60 bp, artificial
  Sequence comprising delta(CT) allele of SNP rs10643535
tcctgtgtcttgctttctctttctctctctctctctgttcctgtctctgtctctggcgtg

SEQ ID NO: 20 - DNA, 62 bp, artificial
  Sequence comprising “CT” allele of SNP rs10643535
tcctgtgtcttgctttctctttctctctctctctctctgttcctgtctctgtctctggcgtg

SEQ ID NO: 21 - DNA, 60 bp, artificial
  Sequence comprising delta(TC) allele of SNP rs34593676
agcgtctcctcctgtgtcttgctttctctttctctctctctctctgttcctgtctctgtc

SEQ ID NO: 22 - DNA, 62 bp, artificial
  Sequence comprising “TC” allele of SNP rs34593676
agcgtctcctcctgtgtcttgctttctctttctctctctctctctctgttcctgtctctgtc

SEQ ID NO: 23 - DNA, 60 bp, artificial
  Sequence comprising delta(T) allele of SNP rs25122122
tcagcgtctcctcctgtgtcttgctttctctttctctctctctctctgttcctgtctctg

SEQ ID NO: 24 - DNA, 61 bp, artificial
  Sequence comprising “T” allele of SNP rs25122122
tcagcgtctcctcctgtgtcttgctttctcttttctctctctctctctgttcctgtctctg

SEQ ID NO: 25 - DNA, 60 bp, artificial
  Sequence comprising delta(A) allele of SNP rs35407108
gcctgggcaacaaaagtgaaactccgtctcaaaaaaaaaaaagacacaaaagggaggttc

SEQ ID NO: 26 - DNA, 61 bp, artificial
  Sequence comprising “A” allele of SNP rs35407108
gcctgggcaacaaaagtgaaactccgtctcaaaaaaaaaaaaagacacaaaagggaggttc

SEQ ID NO: 27 - DNA, 61 bp, artificial
  Sequence comprising SNP rs59211796
gccgagatcacgccattgcactccagcctgrgcaacaaaagtgaaactccgtctcaaaaaa

SEQ ID NO: 28 - DNA, 61 bp, artificial
  Sequence comprising SNP rs62120529
aaaaaagacacaaaccaggcacagtcgctcrtgcctgtaatcccagcactttgggaggccg

SEQ ID NO: 29 - DNA, 61 bp, artificial
  Sequence comprising SNP rs62120528
cttgaggtcaggagttcaataccagcctgamcaacatggcaaaaccctgtctctactagaa

SEQ ID NO: 30 - DNA, 61 bp, artificial
  Sequence comprising SNP rs12983038
ggagggaggattgtttgagcccaggagttcragaccagcctgggcaatatagtgagaccct

SEQ ID NO: 31 - DNA, 61 bp, artificial
  Sequence comprising SNP rs10853727
tttgctgaacatacatcatatgaagaggcaygcttatgatctgcacctgcgtctggagttg

SEQ ID NO: 32 - DNA, 61 bp, artificial
  Sequence comprising SNP rs7254424
aattcttggattacaggcatgatccattgcrcctggcctcattattttcttaaaccgtttt

SEQ ID NO: 33 - DNA, 61 bp, artificial
  Sequence comprising SNP rs1549928
gaagcaaagaaagaggaaacagacagtagaracagggacagagacaatttggaaaccgagt

SEQ ID NO: 34 - DNA, 60 bp, artificial
  Sequence comprising delta(A) allele of SNP rs34347451
gggatggctgccctccaacactcggtttccaaattgtctctgtccctgtttctactgtct

SEQ ID NO: 35 - DNA, 61 bp, artificial
  Sequence comprising “A” allele of SNP rs34347451
gggatggctgccctccaacactcggtttccaaaattgtctctgtccctgtttctactgtct

SEQ ID NO: 36 - DNA, 60 bp, artificial
  Sequence comprising delta(A) allele of SNP rs35814928
tctgggatcccagtcgggtgtgaggacttcaacccgaggttggcctgtgcccgggatggc

SEQ ID NO: 37 - DNA, 61 bp, artificial
  Sequence comprising “A” allele of SNP rs35814928
tctgggatcccagtcgggtgtgaggacttcaaacccgaggttggcctgtgcccgggatggc

SEQ ID NO: 38 - DNA, 61 bp, artificial
  Sequence comprising SNP rs4803222
gagcgtgaaggcacagcacacacagtgggasagagagtgggagccggccccctcctcgcct

SEQ ID NO: 39 - DNA, 60 bp, artificial
  Sequence comprising delta(T) allele of SNP rs11322783
agtgcgagagcaggcagcgccggggggcctctgcgatcaccgtgcacaggacccacagcc

SEQ ID NO: 40 - DNA, 61 bp, artificial
  Sequence comprising “T” allele of SNP rs11322783
agtgcgagagcaggcagcgccggggggccttctgcgatcaccgtgcacaggacccacagcc

SEQ ID NO: 41 - DNA, 61 bp, artificial
  Sequence comprising SNP rs4803221
cagcgtccggggctccagcgagcggtagtgsgagagcaggcagcgccggggggccttctgc

SEQ ID NO: 42 - DNA, 61 bp, artificial
  Sequence comprising SNP rs12979860
tgtactgaaccagggagctccccgaaggcgygaaccagggttgaattgcactccgcgctcc

SEQ ID NO: 43 - DNA, 61 bp, artificial
  Sequence comprising SNP rs12971396
gaagaccacgctggctttgcggcaccgaggsgagtcctggagccagggagggagggcagcg

SEQ ID NO: 44 - DNA, 55 bp, artificial
  Sequence comprising SNP rs11672932
tcgcccggccagcccaatggacgacagsagctgctttcggcagccaatggcgtgg

SEQ ID NO: 45 - DNA, 53 bp, artificial
  Sequence comprising SNP rs11882871
tccctgtagaaggacccgctcctcttrtatctgagacagtggatccaagtcag

SEQ ID NO: 46 - DNA, 53 bp, artificial
  Sequence comprising SNP rs56215543
gatataagaggagcgggtccttctacrgggaagagaccacagttctccaggaa

SEQ ID NO: 47 - DNA, 53 bp, artificial
  Sequence comprising SNP rs12979731
tccagagctcaagttttttcctgccayagcaaccgttggagggtcgtacaatg

SEQ ID NO: 48 - DNA, 53 bp, artificial
  Sequence comprising SNP rs2020358
cgagccagggactcaggtggcctgagkttcagttctgaccctgccagttaatt

SEQ ID NO: 49 - DNA, 53 bp, artificial
  Sequence comprising SNP rs34853289
tcattaagaccatactaggacctcagytggagagtttaaaacgtgatctcaac

SEQ ID NO: 50 - DNA, 53 bp, artificial
  Sequence comprising SNP rs8107030
gggtgccgtctttcttagggaagttcrggcagtggtgaagagcatgggtcttg

SEQ ID NO: 51 - DNA, 32 bp, artificial
  Sequence comprising SNP rs41537748
aggctctgctcaagaytgaggtgtgacgaagg

SEQ ID NO: 52 - DNA, 52 bp, artificial
  Sequence comprising delta(ATAT) allele of SNP rs59702201
gcatatatatatatatatatatatattttgagacagggtcttgttcggtcac

SEQ ID NO: 53 - DNA, 56 bp, artificial
  Sequence comprising “ATAT” allele of SNP rs59702201
gcatatatatatatatatatatatatatattttgagacagggtcttgttcggtcac

SEQ ID NO: 54 - DNA, 53 bp, artificial
  Sequence comprising SNP rs2596806
taagacagggtctcactctgtcactgsagtgcaatggcatgatcacagctcac

SEQ ID NO: 55 - DNA, 53 bp, artificial
  Sequence comprising SNP rs2569377
gtaacctacaggaaggtatgttcccargaggattccacctgctctggttttgt

SEQ ID NO: 56 - DNA, 53 bp, artificial
  Sequence comprising SNP rs4803219
ctgagctccatggggcagcttttatcyctgacagaagggcagtcccagctgat

SEQ ID NO: 57 - DNA, 53 bp, artificial
  Sequence comprising SNP rs28416813
cagagagaaagggagctgagggaatgsagaggctgcccactgagggcaggggc

SEQ ID NO: 58 - DNA, 53 bp, artificial
  Sequence comprising SNP rs630388
agcaccagcactggcatgcagtccccrgtcatgtctgtgtcacagagagaaag

SEQ ID NO: 59 - DNA, 53 bp, artificial
  Sequence of coding strand comprising SNP rs629976
tggagcagttcctgtcgccaggctccrcggggctctcccggatgcaaggggct

SEQ ID NO: 60 - DNA, 53 bp, artificial
  Sequence comprising allele encoding IL28b-His32 of SNP rs629976
tggagcagttcctgtcgccaggctccacggggctctcccggatgcaaggggct

SEQ ID NO: 61 - protein, 17 aa, artificial
  Synthetic Construct
GAVPVARLHGALPDARG

SEQ ID NO: 62 - DNA, 53 bp, artificial
  Sequence comprising allele encoding IL28b-Arg32 of SNP rs629976
tggagcagttcctgtcgccaggctccgcggggctctcccggatgcaaggggct

SEQ ID NO: 63 - protein, 17 aa, artificial
  Synthetic Construct
GAVPVARLRGALPDARG

SEQ ID NO: 64 - DNA, 53 bp, artificial
  Sequence comprising SNP rs629008
cttcaggaaaacatgagtcagtccctrcagtaggagcatgagatagcccactg

SEQ ID NO: 65 - DNA, 53 bp, artificial
  Sequence comprising SNP rs628973
gggaggatggtagaggaccctcttckwmaggaaaacatgagtcagtccctgca

SEQ ID NO: 66 - DNA, 53 bp, artificial
  Sequence of non-coding strand comprising SNP rs8103142
tcctggggaagaggcgggagcggcacytgcagtccttcagcagaagcgactct

SEQ ID NO: 67 - DNA, 53 bp, artificial
  Sequence comprising coding strand of HR allele encoding IL28B-Lys74 of SNP rs8103142
agagtcgcttctgctgaaggactgcaagtgccgctcccgcctcttccccagga

SEQ ID NO: 68 - protein, 17 aa, artificial
  Synthetic Construct
ESLLLKDCKCRSRLFPR

SEQ ID NO: 69 - DNA, 53 bp, artificial
  Sequence comprising coding strand of LR allele encoding IL28B-Arg74 of SNP rs8103142
agagtcgcttctgctgaaggactgcaggtgccgctcccgcctcttccccagga

SEQ ID NO: 70 - protein, 17 aa, artificial
  Synthetic Construct
ESLLLKDCRCRSRLFPR

SEQ ID NO: 71 - DNA, 53 bp, artificial
  Sequence comprising SNP rs8102358
gtgaaggggccactacagagccaggtragcagggctgggagggcaggggtggg

SEQ ID NO: 72 - DNA, 53 bp, artificial
  Sequence comprising SNP rs11881222
agagggcacagccagtgtggtcaggtrggagcagagggaaggggtagcaggtg

SEQ ID NO: 73 - DNA, 53 bp, artificial
  Sequence of coding strand comprising SNP rs61735713
cccggggccgcctccaccattggctgyaccggctccaggaggccccaaaaaag

SEQ ID NO: 74 - DNA, 53 bp, artificial
  Sequence comprising allele encoding IL28B-His160 allele of SNP rs61735713
cccggggccgcctccaccattggctgcaccggctccaggaggccccaaaaaag

SEQ ID NO: 75 - protein, 17 aa, artificial
  Synthetic Construct
RGRLHHWLHRLQEAPKK

SEQ ID NO: 76 - DNA, 53 bp, artificial
  Sequence comprising allele encoding IL28B-Tyr160 allele of SNP rs61735713
cccggggccgcctccaccattggctgtaccggctccaggaggccccaaaaaag

SEQ ID NO: 77 - protein, 17 aa, artificial
  Synthetic Construct
RGRLHHWLYRLQEAPKK

SEQ ID NO: 78 - DNA, 53 bp, artificial
  Sequence of non-coding strand comprising SNP rs62120527
gaagaggttgaaggtgacagaggcctygaggcagccaggggactcctgtaggg

SEQ ID NO: 79 - DNA, 53 bp, artificial
  Sequence comprising coding strand of allele encoding IL28B-Glu175 allele of SNP rs62120527
ccctacaggagtcccctggctgcctcgaggcctctgtcaccttcaacctcttc

SEQ ID NO: 80 - protein, 17 aa, artificial
  Synthetic Construct
LQESPGCLEASVTFNLF

SEQ ID NO: 81 - DNA, 53 bp, artificial
  Sequence comprising coding strand of allele encoding IL28B-Lys175 allele of SNP rs62120527
ccctacaggagtcccctggctgcctcaaggcctctgtcaccttcaacctcttc

SEQ ID NO: 82 - protein, 17 aa, artificial
  Synthetic Construct
LQESPGCLKASVTFNLF

SEQ ID NO: 83 - DNA, 52 bp, artificial
  Sequence comprising SNP rs4803217
tagcgactgggtgacaataaattaagmcaagtggctaatttataaataaaat

SEQ ID NO: 84 - DNA, 61 bp, artificial
  Sequence comprising SNP rs8105790
ttcccttcctgacatcactccaatgtcctgyttctgtggttacatcttccgctaatgatgc

SEQ ID NO: 85 - DNA, 61 bp, artificial
  Sequence comprising HR allele of SNP rs8105790
ttcccttcctgacatcactccaatgtcctgtttctgtggttacatcttccgctaatgatgc

SEQ ID NO: 86 - DNA, 61 bp, artificial
  Sequence comprising LR allele of SNP rs8105790
ttcccttcctgacatcactccaatgtcctgcttctgtggttacatcttccgctaatgatgc

SEQ ID NO: 87 - DNA, 52 bp, artificial
  Sequence comprising SNP rs12980275
ctgagagaagtcaaattcctagaaacrgacgtgtctaaatatttgccggggt

SEQ ID NO: 88 - DNA, 52 bp, artificial
  Sequence comprising HR allele of SNP rs12980275
ctgagagaagtcaaattcctagaaacagacgtgtctaaatatttgccggggt

SEQ ID NO: 89 - DNA, 52 bp, artificial
  Sequence comprising LR allele of SNP rs12980275
ctgagagaagtcaaattcctagaaacggacgtgtctaaatatttgccggggt

SEQ ID NO: 90 - DNA, 52 bp, artificial
  Sequence comprising SNP rs7750468
taaatgaaatttggaaaacaatccagraacaaaatgagaaaatagacaaaga

SEQ ID NO: 91 - DNA, 52 bp, artificial
  Sequence comprising HR allele of SNP rs7750468
taaatgaaatttggaaaacaatccagaaacaaaatgagaaaatagacaaaga

SEQ ID NO: 92 - DNA, 52 bp, artificial
  Sequence comprising LR allele of SNP rs7750468
taaatgaaatttggaaaacaatccaggaacaaaatgagaaaatagacaaaga

SEQ ID NO: 93 - DNA, 52 bp, artificial
  Sequence comprising SNP rs2746200
ggagggtcactgtgattcagtgatgcycaactccctaagagtcttaccaaaa

SEQ ID NO: 94 - DNA, 52 bp, artificial
  Sequence comprising HR allele of SNP rs2746200
ggagggtcactgtgattcagtgatgcccaactccctaagagtcttaccaaaa

SEQ ID NO: 95 - DNA, 52 bp, artificial
  Sequence comprising LR allele of SNP rs2746200
ggagggtcactgtgattcagtgatgctcaactccctaagagtcttaccaaaa

SEQ ID NO: 96 - DNA, 52 bp, artificial
  Sequence comprising SNP rs927188
ttgtagaaattgagcaggttgtagatmtaatcacccggtgggttcttcctgc

SEQ ID NO: 97 - DNA, 52 bp, artificial
  Sequence comprising HR allele of SNP rs927188
ttgtagaaattgagcaggttgtagatataatcacccggtgggttcttcctgc

SEQ ID NO: 98 - DNA, 52 bp, artificial
  Sequence comprising LR allele of SNP rs927188
ttgtagaaattgagcaggttgtagatctaatcacccggtgggttcttcctgc

SEQ ID NO: 99 - DNA, 52 bp, artificial
  Sequence comprising SNP rs2517861
tgatatttcttcatgggatggtctccrtgatacaatggtaagggaaaacagc

SEQ ID NO: 100 - DNA, 52 bp, artificial
  Sequence comprising LR allele of SNP rs2517861
tgatatttcttcatgggatggtctccatgatacaatggtaagggaaaacagc

SEQ ID NO: 101 - DNA, 52 bp, artificial
  Sequence comprising HR allele of SNP rs2517861
tgatatttcttcatgggatggtctccgtgatacaatggtaagggaaaacagc

SEQ ID NO: 102 - DNA, 52 bp, artificial
  Sequence comprising SNP rs2025503
catacactgtacaaagattttcacttmaccaagttggaggactcacttgatc

SEQ ID NO: 103 - DNA, 52 bp, artificial
  Sequence comprising LR allele of SNP rs2025503
catacactgtacaaagattttcacttaaccaagttggaggactcacttgatc

SEQ ID NO: 104 - DNA, 52 bp, artificial
  Sequence comprising HR allele of SNP rs2025503
catacactgtacaaagattttcacttcaccaagttggaggactcacttgatc

SEQ ID NO: 105 - DNA, 52 bp, artificial; identical to SEQ ID NO: 102
  Sequence comprising SNP rs2066911

SEQ ID NO: 106 - DNA, 52 bp, artificial; identical to SEQ ID NO: 103
  Sequence comprising LR allele of SNP rs2066911

SEQ ID NO: 107 - DNA, 52 bp, artificial; identical to SEQ ID NO: 104
  Sequence comprising HR allele of SNP rs2066911

SEQ ID NO: 108 - DNA, 61 bp, artificial
  Sequence comprising SNP rs10018218
atgggctcaaatctcatatccttcctccaayacgtgttaaaactcaggccctttggtgact

SEQ ID NO: 109 - DNA, 61 bp, artificial
  Sequence comprising HR allele of SNP rs10018218
atgggctcaaatctcatatccttcctccaacacgtgttaaaactcaggccctttggtgact

SEQ ID NO: 110 - DNA, 61 bp, artificial
  Sequence comprising LR allele of SNP rs10018218
atgggctcaaatctcatatccttcctccaatacgtgttaaaactcaggccctttggtgact

SEQ ID NO: 111 - DNA, 61 bp, artificial
  Sequence comprising SNP rs1581096
aaaagagtacaagggatccattttccccatrtccttactaatacttgctatcatttgtctt

SEQ ID NO: 112 - DNA, 61 bp, artificial
  Sequence comprising LR allele of SNP rs1581096
aaaagagtacaagggatccattttccccatatccttactaatacttgctatcatttgtctt

SEQ ID NO: 113 - DNA, 61 bp, artificial
  Sequence comprising HR allele of SNP rs1581096
aaaagagtacaagggatccattttccccatgtccttactaatacttgctatcatttgtctt

SEQ ID NO: 114 - DNA, 61 bp, artificial
  Sequence comprising SNP rs1250105
aaaatcagccaaagcctgcagctaatcctgrgactggccaggtgacctcacaggagcgcct

SEQ ID NO: 115 - DNA, 61 bp, artificial
  Sequence comprising LR allele of SNP rs1250105
aaaatcagccaaagcctgcagctaatcctgagactggccaggtgacctcacaggagcgcct

SEQ ID NO: 116 - DNA, 61 bp, artificial
  Sequence comprising HR allele of SNP rs1250105
aaaatcagccaaagcctgcagctaatcctgggactggccaggtgacctcacaggagcgcct

SEQ ID NO: 117 - DNA, 61 bp, artificial
  Sequence comprising SNP rs1939565
gcaaagcactggcactttattatatttaccraaagtacttttggggagagaactaccctat

SEQ ID NO: 118 - DNA, 61 bp, artificial
  Sequence comprising HR allele of SNP rs1939565
gcaaagcactggcactttattatatttaccaaaagtacttttggggagagaactaccctat

SEQ ID NO: 119 - DNA, 61 bp, artificial
  Sequence comprising LR allele of SNP rs1939565
gcaaagcactggcactttattatatttaccgaaagtacttttggggagagaactaccctat

SEQ ID NO: 120 - DNA, 61 bp, artificial
  Sequence comprising SNP rs568910
ctgagtgcaaggggtctgtaggcacttatgkagttgtaaagtcacatgaagctttaaggtt

SEQ ID NO: 121 - DNA, 61 bp, artificial
  Sequence comprising HR allele of SNP rs568910
ctgagtgcaaggggtctgtaggcacttatggagttgtaaagtcacatgaagctttaaggtt

SEQ ID NO: 122 - DNA, 61 bp, artificial
  Sequence comprising LR allele of SNP rs568910
ctgagtgcaaggggtctgtaggcacttatgtagttgtaaagtcacatgaagctttaaggtt

SEQ ID NO: 123 - DNA, 61 bp, artificial
  Sequence comprising SNP rs557905
ccactttgggaatgcacatttagatatttcrtttccaaatcccaatcactcccctctaccc

SEQ ID NO: 124 - DNA, 61 bp, artificial
  Sequence comprising LR allele of SNP rs557905
ccactttgggaatgcacatttagatatttcatttccaaatcccaatcactcccctctaccc

SEQ ID NO: 125 - DNA, 61 bp, artificial
  Sequence comprising HR allele of SNP rs557905
ccactttgggaatgcacatttagatatttcgtttccaaatcccaatcactcccctctaccc

SEQ ID NO: 126 - DNA, 61 bp, artificial
  Sequence comprising SNP rs6806020
aaaaaaccacacactcaccacattggtgtcyagtctcaggccacagccccacactcccagt

SEQ ID NO: 127 - DNA, 61 bp, artificial
  Sequence comprising LR allele of SNP rs6806020
aaaaaaccacacactcaccacattggtgtccagtctcaggccacagccccacactcccagt

SEQ ID NO: 128 - DNA, 61 bp, artificial
  Sequence comprising HR allele of SNP rs6806020
aaaaaaccacacactcaccacattggtgtctagtctcaggccacagccccacactcccagt

SEQ ID NO: 129 - DNA, 61 bp, artificial
  Sequence comprising SNP rs12486361
aatagatagaagtgacaaaacctctgccttygtggagctaacaatctaataggaggagaaa

SEQ ID NO: 130 - DNA, 61 bp, artificial
  Sequence comprising HR allele of SNP rs12486361
aatagatagaagtgacaaaacctctgccttcgtggagctaacaatctaataggaggagaaa

SEQ ID NO: 131 - DNA, 61 bp, artificial
  Sequence comprising LR allele of SNP rs12486361
aatagatagaagtgacaaaacctctgcctttgtggagctaacaatctaataggaggagaaa

SEQ ID NO: 132 - DNA, 61 bp, artificial
  Sequence comprising SNP rs10283103
agttctttattaataagtcacagcatcctgyaaggaagaaattgtgcatcagctgccaagc

SEQ ID NO: 133 - DNA, 61 bp, artificial
  Sequence comprising HR allele of SNP rs10283103
agttctttattaataagtcacagcatcctgcaaggaagaaattgtgcatcagctgccaagc

SEQ ID NO: 134 - DNA, 61 bp, artificial
  Sequence comprising LR allele of SNP rs10283103
agttctttattaataagtcacagcatcctgtaaggaagaaattgtgcatcagctgccaagc

SEQ ID NO: 135 - DNA, 61 bp, artificial
  Sequence comprising SNP rs2114487
aggacactggaaaagggatagaaacagattytcccccggggccttcagaactgaaagtagt

SEQ ID NO: 136 - DNA, 61 bp, artificial
  Sequence comprising HR allele of SNP rs2114487
aggacactggaaaagggatagaaacagattctcccccggggccttcagaactgaaagtagt

SEQ ID NO: 137 - DNA, 61 bp, artificial
  Sequence comprising LR allele of SNP rs2114487
aggacactggaaaagggatagaaacagattttcccccggggccttcagaactgaaagtagt

SEQ ID NO: 138 - DNA, 61 bp, artificial
  Sequence comprising SNP rs7196702
ttcatagctgtcttgcccctcctgtggtctrtaagaatgggaccaggactcctagttgtga

SEQ ID NO: 139 - DNA, 61 bp, artificial
  Sequence comprising HR allele of SNP rs7196702
ttcatagctgtcttgcccctcctgtggtctataagaatgggaccaggactcctagttgtga

SEQ ID NO: 140 - DNA, 61 bp, artificial
  Sequence comprising LR allele of SNP rs7196702
ttcatagctgtcttgcccctcctgtggtctgtaagaatgggaccaggactcctagttgtga

SEQ ID NO: 141 - DNA, 61 bp, artificial
  Sequence comprising SNP rs3093390
gttgggaagagatatgcacaatctgccctcytggctggtatgagtgagtcccagctcaccg

SEQ ID NO: 142 - DNA, 61 bp, artificial
  Sequence comprising LR allele of SNP rs3093390
gttgggaagagatatgcacaatctgccctcctggctggtatgagtgagtcccagctcaccg

SEQ ID NO: 143 - DNA, 61 bp, artificial
  Sequence comprising HR allele of SNP rs3093390
gttgggaagagatatgcacaatctgccctcttggctggtatgagtgagtcccagctcaccg

SEQ ID NO: 144 - DNA, 61 bp, artificial
  Sequence comprising SNP rs7512595
agaccaaatgcattaatacatatgcaaagcrtttggaacagctggcatatataagtgccat

SEQ ID NO: 145 - DNA, 61 bp, artificial
  Sequence comprising LR allele of SNP rs7512595
agaccaaatgcattaatacatatgcaaagcatttggaacagctggcatatataagtgccat

SEQ ID NO: 146 - DNA, 61 bp, artificial
  Sequence comprising HR allele of SNP rs7512595
agaccaaatgcattaatacatatgcaaagcgtttggaacagctggcatatataagtgccat

SEQ ID NO: 147 - DNA, 61 bp, artificial
  Sequence comprising SNP rs1002960
cggcccttgtctgcgtacccctagacttctmattatgtaagaaaaataaccactatttggt

SEQ ID NO: 148 - DNA, 61 bp, artificial
  Sequence comprising HR allele of SNP rs1002960
cggcccttgtctgcgtacccctagacttctaattatgtaagaaaaataaccactatttggt

SEQ ID NO: 149 - DNA, 61 bp, artificial
  Sequence comprising LR allele of SNP rs1002960
cggcccttgtctgcgtacccctagacttctcattatgtaagaaaaataaccactatttggt

SEQ ID NO: 150 - DNA, 61 bp, artificial
  Sequence comprising SNP rs1931704
taggaggaaacgtgtgaagagggcttgggtractctaagacagttacctcatgacaaagaa

SEQ ID NO: 151 - DNA, 61 bp, artificial
  Sequence comprising LR allele of SNP rs1931704
taggaggaaacgtgtgaagagggcttgggtaactctaagacagttacctcatgacaaagaa

SEQ ID NO: 152 - DNA, 61 bp, artificial
  Sequence comprising HR allele of SNP rs1931704
taggaggaaacgtgtgaagagggcttgggtgactctaagacagttacctcatgacaaagaa

SEQ ID NO: 153 - DNA, 61 bp, artificial
  Sequence comprising SNP rs66616
gaaaaacaagaaagctggtttctttgatttracagacaatgtatagaccatttgggcactg

SEQ ID NO: 154 - DNA, 61 bp, artificial
  Sequence comprising LR allele of SNP rs66616
gaaaaacaagaaagctggtttctttgatttaacagacaatgtatagaccatttgggcactg

SEQ ID NO: 155 - DNA, 61 bp, artificial
  Sequence comprising HR allele of SNP rs66616
gaaaaacaagaaagctggtttctttgatttgacagacaatgtatagaccatttgggcactg

SEQ ID NO: 156 - DNA, 61 bp, artificial
  Sequence comprising SNP rs4402825
gtttgtggatcccttggattctgtctgctayacagcaaccagaatggctaacattaaagaa

SEQ ID NO: 157 - DNA, 61 bp, artificial
  Sequence comprising LR allele of SNP rs4402825
gtttgtggatcccttggattctgtctgctacacagcaaccagaatggctaacattaaagaa

SEQ ID NO: 158 - DNA, 61 bp, artificial
  Sequence comprising HR allele of SNP rs4402825
gtttgtggatcccttggattctgtctgctatacagcaaccagaatggctaacattaaagaa